SEQUENCE LISTING (635 sequences)

SEQ ID NO: 1 - DNA, 144 bp, synthetic construct
  misc_feature 1..144: note = Description of Artificial Sequence: Synthetic polynucleotide
  source 1..144: mol_type = other DNA
gctctgcttcattcctgtctgaagaagggcagatagtttggctgctcctgtgytgtcacctgcaattctcccttatcagggccattggcctctcccttctctctgtgagggatattttctctgacttgtcaatccacatcttcc

SEQ ID NO: 2 - DNA, 82 bp, synthetic construct
  misc_feature 1..82: note = Description of Artificial Sequence: Synthetic oligonucleotide
  source 1..82: mol_type = other DNA
ggcttgcaatggctccaaccggaagggcggtgctcgagctgtggtgcgtgcygctaagttgtgcgttccagggtgcactcgc

SEQ ID NO: 3 - DNA, 65 bp, synthetic construct
  misc_feature 1..65: note = Description of Artificial Sequence: Synthetic oligonucleotide
  source 1..65: mol_type = other DNA
gcaactatacccttgatggatggagatttaygcaatgtgttttactgggtagagtgacagacctt

SEQ ID NO: 4 - DNA, 124 bp, synthetic construct
  misc_feature 1..124: note = Description of Artificial Sequence: Synthetic polynucleotide
  source 1..124: mol_type = other DNA
cctgaacttatttggcaagagcgatgagtactcttaaaattactatctggaaattatattatttagaatctgccaattacctagatcccccctsaacaattgtttcaccaaggaacttcctgaa

SEQ ID NO: 5 - DNA, 180 bp, synthetic construct
  misc_feature 1..180: note = Description of Artificial Sequence: Synthetic polynucleotide
  source 1..180: mol_type = other DNA
gaattggttgtctccttatgggaactggaagtattttgacakctttaccacatttcttcatgggatagtaagtgttaaacagctctgagccatttattatcagctacttgtaaattagcagtagaattttatttttatacttgtaagtgggcagttaccttttgagaggaatacctatag

SEQ ID NO: 6 - DNA, 87 bp, synthetic construct
  misc_feature 1..87: note = Description of Artificial Sequence: Synthetic oligonucleotide
  source 1..87: mol_type = other DNA
gcggagggaagctcatcagtggggccacgagctgagtgcgtcctgtcactccactcccatgtcccttgggaaggtctgagactaggg

SEQ ID NO: 7 - DNA, 192 bp, synthetic construct
  misc_feature 1..192: note = Description of Artificial Sequence: Synthetic polynucleotide
  source 1..192: mol_type = other DNA
tactacacctcagatatatttcttcatgaagacctcacagtaaaaataggtgattttggtctagctacagwgaaatctcgatggagtgggtcccatcagtttgaacagttgtctggatccattttgtggatggtaagaattgaggctatttttccactgattaaatttttggccctgagatgctgctgagtt

SEQ ID NO: 8 - DNA, 198 bp, synthetic construct
  misc_feature 1..198: note = Description of Artificial Sequence: Synthetic polynucleotide
  source 1..198: mol_type = other DNA
tgctaatactgtttcgtatttatagctgatttgatggagttggacatggccatggaaccagacagaaaagcggctgttagtcactggcagcaacagtcttacctggactctggaatccattctggtgccactrccacagctccttctctgagtggtaaaggcaatcctgaggaagaggatgtggatacctcccaagtc

SEQ ID NO: 9 - DNA, 184 bp, synthetic construct
  misc_feature 1..184: note = Description of Artificial Sequence: Synthetic polynucleotide
  source 1..184: mol_type = other DNA
tttgatggagttggacatggccatggaaccagacagaaaagcggctgttagtcactggcagcaacagtcttacctggactctggaatccattctggtgccactaccacagctccttytctgagtggtaaaggcaatcctgaggaagaggatgtggatacctcccaagtcctgtatgagtgggaa

SEQ ID NO: 10 - DNA, 181 bp, synthetic construct
  misc_feature 1..181: note = Description of Artificial Sequence: Synthetic polynucleotide
  source 1..181: mol_type = other DNA
gtggacaacccccacgtgtgccgcctgctgggcatctgcctcacctccaccgtgcagctcatcaygcagctcatgcccttcggctgcctcctggactatgtccgggaacacaaagacaatattggctcccagtacctgctcaactggtgtgtgcagatcgcaaaggtaatcagggaaggga

SEQ ID NO: 11 - DNA, 166 bp, synthetic construct
  misc_feature 1..166: note = Description of Artificial Sequence: Synthetic polynucleotide
  source 1..166: mol_type = other DNA
gcatgaactacttggaggaccgtcgcttggtgcaccgcgacctggcagccaggaacgtactggtgaaaacaccgcagcatgtcaagatcacagattttgggckggccaaactgctgggtgcggaagagaaagaataccatgcagaaggaggcaaagtaaggaggtg

SEQ ID NO: 12 - DNA, 172 bp, synthetic construct
  misc_feature 1..172: note = Description of Artificial Sequence: Synthetic polynucleotide
  source 1..172: mol_type = other DNA
caggattcctacaggaagcaagtagtaattgatggagaaacctgtctcttggatattctcgacacagsaggtcaagaggagtacagtgcaatgagggaccagtacatgaggactggggagggctttctttgtgtatttgccataaataatactaaatcatttgaagatattc

SEQ ID NO: 13 - DNA, 207 bp, synthetic construct
  misc_feature 1..207: note = Description of Artificial Sequence: Synthetic polynucleotide
  source 1..207: mol_type = other DNA
acaggaagcaagtagtaattgatggagaaacctgtctcttggatattctcgacacagcaggtcamgaggagtacagtgcaatgagggaccagtacatgaggactggggagggctttctttgtgtatttgccataaataatactaaatcatttgaagatattcaccattataggtgggtttaaattgaatataataagctgacattaa

SEQ ID NO: 14 - DNA, 169 bp, synthetic construct
  misc_feature 1..169: note = Description of Artificial Sequence: Synthetic polynucleotide
  source 1..169: mol_type = other DNA
tattaaccttatgtgtgacatgttctaatatagtcacattttcattatttttattataaggcctgctgaaaatgactgaatataaacttgtggtagttggagctrgtggcgtaggcaagagtgccttgacgatacagctaattcagaatcattttgtggacgaatatga

SEQ ID NO: 15 - DNA, 171 bp, synthetic construct
  misc_feature 1..171: note = Description of Artificial Sequence: Synthetic polynucleotide
  source 1..171: mol_type = other DNA
tattaaccttatgtgtgacatgttctaatatagtcacattttcattatttttattataaggcctgctgaaaatgactgaatataaacttgtggtagttggagctgrtggcgtaggcaagagtgccttgacgatacagctaattcagaatcattttgtggacgaatatgatc

SEQ ID NO: 16 - DNA, 230 bp, synthetic construct
  misc_feature 1..230: note = Description of Artificial Sequence: Synthetic polynucleotide
  source 1..230: mol_type = other DNA
cattatttttattataaggcctgctgaaaatgactgaatataaacttgtggtagttggagctggtgrcgtaggcaagagtgccttgacgatacagctaattcagaatcattttgtggacgaatatgatccaacaatagaggtaaatcttgttttaatatgcatattactggtgcaggaccattctttgatacagataaaggtttctctgaccattttcatgagtacttat

SEQ ID NO: 17 - DNA, 210 bp, synthetic construct
  misc_feature 1..210: note = Description of Artificial Sequence: Synthetic polynucleotide
  source 1..210: mol_type = other DNA
attcttacagaaaacaagtggttatagatggtgaaacctgtttgttggacatactggatacagctggamaagaagagtacagtgccatgagagaccaatacatgaggacaggcgaaggcttcctctgtgtatttgccatcaataatagcaagtcatttgcggatattaacctctacaggtactaggagcattattttctctgaaaggatg

SEQ ID NO: 18 - DNA, 206 bp, synthetic construct
  misc_feature 1..206: note = Description of Artificial Sequence: Synthetic polynucleotide
  source 1..206: mol_type = other DNA
ttacagaaaacaagtggttatagatggtgaaacctgtttgttggacatactggatacagctggacawgaagagtacagtgccatgagagaccaatacatgaggacaggcgaaggcttcctctgtgtatttgccatcaataatagcaagtcatttgcggatattaacctctacaggtactaggagcattattttctctgaaaggatg

SEQ ID NO: 19 - DNA, 162 bp, synthetic construct
  misc_feature 1..162: note = Description of Artificial Sequence: Synthetic polynucleotide
  source 1..162: mol_type = other DNA
tggtttccaacaggttcttgctggtgtgaaatgactgagtacaaactggtggtggttggagcagrtggtgttgggaaaagcgcactgacaatccagctaatccagaaccactttgtagatgaatatgatcccaccatagaggtgaggcccagtggtagcccg

SEQ ID NO: 20 - DNA, 158 bp, synthetic construct
  misc_feature 1..158: note = Description of Artificial Sequence: Synthetic polynucleotide
  source 1..158: mol_type = other DNA
tttccaacaggttcttgctggtgtgaaatgactgagtacaaactggtggtggttggagcaggtgrtgttgggaaaagcgcactgacaatccagctaatccagaaccactttgtagatgaatatgatcccaccatagaggtgaggcccagtggtagccc

SEQ ID NO: 21 - DNA, 189 bp, synthetic construct
  misc_feature 1..189: note = Description of Artificial Sequence: Synthetic polynucleotide
  source 1..189: mol_type = other DNA
ggcacccgcgtccgcgccatggccatctacaagcagtcacagcacatgacggaggttgtgaggcrctgcccccaccatgagcgctgctcagatagcgatggtgagcagctggggctggagagacgacagggctggttgcccagggtccccaggcctctgattcctcactgattgctcttaggtctggcc

SEQ ID NO: 22 - DNA, 165 bp, synthetic construct
  misc_feature 1..165: note = Description of Artificial Sequence: Synthetic polynucleotide
  source 1..165: mol_type = other DNA
cctcctcagcatcttatccgagtggaaggaaatttgcgtgtggagtatttggatgacagaaacacttttygacatagtgtggtggtgccctatgagccgcctgaggtctggtttgcaactggggtctctgggaggaggggttaagggtggttgtcagtggccctc

SEQ ID NO: 23 - DNA, 183 bp, synthetic construct
  misc_feature 1..183: note = Description of Artificial Sequence: Synthetic polynucleotide
  source 1..183: mol_type = other DNA
cttgggcctgtgttatctcctaggttggctctgactgtaccaccatccactacaactacatgtgtaacagtkcctgcatgggcggcatgaaccggaggcccatcctcaccatcatcacactggaagactccaggtcaggagccacttgccaccctgcacactggcctgctgtgccccagcctc

SEQ ID NO: 24 - DNA, 163 bp, synthetic construct
  misc_feature 1..163: note = Description of Artificial Sequence: Synthetic polynucleotide
  source 1..163: mol_type = other DNA
taggttggctctgactgtaccaccatccactacaactacatgtgtaacagttcctgcatgggcrgcatgaaccggaggcccatcctcaccatcatcacactggaagactccaggtcaggagccacttgccaccctgcacactggcctgctgtgccccagcctc

SEQ ID NO: 25 - DNA, 163 bp, synthetic construct
  misc_feature 1..163: note = Description of Artificial Sequence: Synthetic polynucleotide
  source 1..163: mol_type = other DNA
ctgactgtaccaccatccactacaactacatgtgtaacagttcctgcatgggcggcatgaacyggaggcccatcctcaccatcatcacactggaagactccaggtcaggagccacttgccaccctgcacactggcctgctgtgccccagcctctgcttgcctc

SEQ ID NO: 26 - DNA, 162 bp, synthetic construct
  misc_feature 1..162: note = Description of Artificial Sequence: Synthetic polynucleotide
  source 1..162: mol_type = other DNA
tgactgtaccaccatccactacaactacatgtgtaacagttcctgcatgggcggcatgaaccrgaggcccatcctcaccatcatcacactggaagactccaggtcaggagccacttgccaccctgcacactggcctgctgtgccccagcctctgcttgcctc

SEQ ID NO: 27 - DNA, 176 bp, synthetic construct
  misc_feature 1..176: note = Description of Artificial Sequence: Synthetic polynucleotide
  source 1..176: mol_type = other DNA
cctcttgcttctcttttcctatcctgagtagtggtaatctactgggacggaacagctttgaggtgygtgtttgtgcctgtcctgggagagaccggcgcacagaggaagagaatctccgcaagaaaggggagcctcaccacgagctgcccccagggagcactaagcgaggtaagcaa

SEQ ID NO: 28 - DNA, 601 bp, Homo sapiens
  source 1..601: mol_type = genomic DNA
agaaaataactaagggaaggaggaaagtggggaggaaggaagaacagtgtgaagacaatggcctgaaaactgaaaaagtctgttaaagttaattatcagtttttgagtccaagaactggctttgctactttctgtaagtttctaatttactgaataagcatgaaaaagattgctttgaggaatggttataaacacattcttagagcatagtaagcagtagggagtaacaaaataacactgattagaatactttactctacttaattaatcaatcatatttagtttgactcaccttcccaghaccttctagttctttcttatctttcagtgcttgtccagacaacattttcatttcaacaactcctgctattgcaatgatgggtacaattgctaagagtaacagtgttagttgccaaccatagatgaaggatataattattcctgtcccaagatttgctatattctgggtaattacagcaagcctggaacctatagcctgcaaaacaaaacaaattagagaaattttaaaaatattatcttcacaactcatgcttctattttctgaaaactcaccttcatgagactatattcattattttat

SEQ ID NO: 29 - DNA, 27 bp, synthetic construct
  misc_feature 1..27: note = Description of Artificial Sequence: Synthetic primer
  source 1..27: mol_type = other DNA
atatttagtttgactcaccttcccagc

SEQ ID NO: 30 - DNA, 19 bp, synthetic construct
  misc_feature 1..19: note = Description of Artificial Sequence: Synthetic primer
  source 1..19: mol_type = other DNA
accactcacctttcccagc

SEQ ID NO: 31 - DNA, 27 bp, synthetic construct
  misc_feature 1..27: note = Description of Artificial Sequence: Synthetic primer
  source 1..27: mol_type = other DNA
atatttagtttgactcaccttcccaga

SEQ ID NO: 32 - DNA, 19 bp, synthetic construct
  misc_feature 1..19: note = Description of Artificial Sequence: Synthetic primer
  source 1..19: mol_type = other DNA
accactcacctttcccaga

SEQ ID NO: 33 - DNA, 27 bp, synthetic construct
  misc_feature 1..27: note = Description of Artificial Sequence: Synthetic primer
  source 1..27: mol_type = other DNA
atatttagtttgactcaccttcccagt

SEQ ID NO: 34 - DNA, 19 bp, synthetic construct
  misc_feature 1..19: note = Description of Artificial Sequence: Synthetic primer
  source 1..19: mol_type = other DNA
accactcacctttcccagt

SEQ ID NO: 35 - DNA, 18 bp, synthetic construct
  misc_feature 1..18: note = Description of Artificial Sequence: Synthetic probe
  source 1..18: mol_type = other DNA
tggacaagcactgaaaga

SEQ ID NO: 36 - DNA, 27 bp, synthetic construct
  misc_feature 1..27: note = Description of Artificial Sequence: Synthetic primer
  source 1..27: mol_type = other DNA
gcaggagttgttgaaatgaaaatgttg

SEQ ID NO: 37 - DNA, 163 bp, synthetic construct
  misc_feature 1..163: note = Description of Artificial Sequence: Synthetic polynucleotide
  source 1..163: mol_type = other DNA
tgactgaatataaacttgtggtagttggagctgstggcgtaggcaagagtgccttgacgatacagctaattcagaatcattttgtggacgaatatgatccaacaatagaggtaaatcttgttttaatatgcatattactggtgcaggaccattctttgataca

SEQ ID NO: 38 - DNA, 163 bp, synthetic construct
  misc_feature 1..163: note = Description of Artificial Sequence: Synthetic polynucleotide
  source 1..163: mol_type = other DNA
tgactgaatataaacttgtggtagttggagctsgtggcgtaggcaagagtgccttgacgatacagctaattcagaatcattttgtggacgaatatgatccaacaatagaggtaaatcttgttttaatatgcatattactggtgcaggaccattctttgataca

SEQ ID NO: 39 - DNA, 163 bp, synthetic construct
  misc_feature 1..163: note = Description of Artificial Sequence: Synthetic polynucleotide
  source 1..163: mol_type = other DNA
tgactgaatataaacttgtggtagttggagctgrtggcgtaggcaagagtgccttgacgatacagctaattcagaatcattttgtggacgaatatgatccaacaatagaggtaaatcttgttttaatatgcatattactggtgcaggaccattctttgataca

SEQ ID NO: 40 - DNA, 163 bp, synthetic construct
  misc_feature 1..163: note = Description of Artificial Sequence: Synthetic polynucleotide
  source 1..163: mol_type = other DNA
tgactgaatataaacttgtggtagttggagctrgtggcgtaggcaagagtgccttgacgatacagctaattcagaatcattttgtggacgaatatgatccaacaatagaggtaaatcttgttttaatatgcatattactggtgcaggaccattctttgataca

SEQ ID NO: 41 - DNA, 163 bp, synthetic construct
  misc_feature 1..163: note = Description of Artificial Sequence: Synthetic polynucleotide
  source 1..163: mol_type = other DNA
tgactgaatataaacttgtggtagttggagctggtgrcgtaggcaagagtgccttgacgatacagctaattcagaatcattttgtggacgaatatgatccaacaatagaggtaaatcttgttttaatatgcatattactggtgcaggaccattctttgataca

SEQ ID NO: 42 - DNA, 164 bp, synthetic construct
  misc_feature 1..164: note = Description of Artificial Sequence: Synthetic polynucleotide
  variation 34..35: note = n is a, c, t, g, unknown or other
  source 1..164: mol_type = other DNA
gtgagtttgtattaaaaggtactggtggagtatnngatagtgtattaaccttatgtgtgacatgttctaatatagtcacattttcattatttttattataaggcctgctgaaaatgactgaatataaacttgtggtagttggagctkgtggcgtaggcaagagt

SEQ ID NO: 43 - DNA, 111 bp, synthetic construct
  misc_feature 1..111: note = Description of Artificial Sequence: Synthetic polynucleotide
  source 1..111: mol_type = other DNA
atatttcttcatgaagacctcacagtaaaaataggtgattttggtctagctacagwgaaatctcgatggagtgggtcccatcagtttgaacagttgtctggatccattttg

SEQ ID NO: 44 - DNA, 111 bp, synthetic construct
  misc_feature 1..111: note = Description of Artificial Sequence: Synthetic polynucleotide
  variation 76: note = n is a, c, t, g, unknown or other
  variation 83: note = n is a, c, t, g, unknown or other
  source 1..111: mol_type = other DNA
gtatccacatcctcttcctcaggattgcctttaccactcagagaaggagctgtggragtggcaccagaatggattncagagtncaggtaagactgttgctgccagtgacta

SEQ ID NO: 45 - DNA, 111 bp, synthetic construct
  misc_feature 1..111: note = Description of Artificial Sequence: Synthetic polynucleotide
  variation 89: note = n is a, c, t, g, unknown or other
  variation 96: note = n is a, c, t, g, unknown or other
  source 1..111: mol_type = other DNA
caggacttgggaggtatccacatcctcttcctcaggattgcctttaccactcagayaaggagctgtggtagtggcaccagaatggattncagagtncaggtaagactgttg

SEQ ID NO: 46 - DNA, 111 bp, synthetic construct
  misc_feature 1..111: note = Description of Artificial Sequence: Synthetic polynucleotide
  source 1..111: mol_type = other DNA
cccacgtgtgccgcctgctgggcatctgcctcacctccaccgtgcagctcatcaygcagctcatgcccttcggctgcctcctggactatgtccgggaacacaaagacaata

SEQ ID NO: 47 - DNA, 111 bp, synthetic construct
  misc_feature 1..111: note = Description of Artificial Sequence: Synthetic polynucleotide
  source 1..111: mol_type = other DNA
ttactttgcctccttctgcatggtattctttctcttccgcacccagcagtttggcckgcccaaaatctgtgatcttgacatgctgcggtgttttcaccagtacgttcctgg

SEQ ID NO: 48 - DNA, 111 bp, synthetic construct
  misc_feature 1..111: note = Description of Artificial Sequence: Synthetic polynucleotide
  variation 63: note = n is a, c, t, g, unknown or other
  source 1..111: mol_type = other DNA
aggaagcaagtagtaattgatggagaaacctgtctcttggatattctcgacacagsaggtcangaggagtacagtgcaatgagggaccagtacatgaggactggggagggc

SEQ ID NO: 49 - DNA, 111 bp, synthetic construct
  misc_feature 1..111: note = Description of Artificial Sequence: Synthetic polynucleotide
  source 1..111: mol_type = other DNA
aagtagtaattgatggagaaacctgtctcttggatattctcgacacagcaggtcamgaggagtacagtgcaatgagggaccagtacatgaggactggggagggctttcttt

SEQ ID NO: 50 - DNA, 111 bp, synthetic construct
  misc_feature 1..111: note = Description of Artificial Sequence: Synthetic polynucleotide
  source 1..111: mol_type = other DNA
ccacaaaatgattctgaattagctgtatcgtcaaggcactcttgcctacgccacragctccaactaccacaagtttatattcagtcattttcagcaggccttataataaaa

SEQ ID NO: 51 - DNA, 111 bp, synthetic construct
  misc_feature 1..111: note = Description of Artificial Sequence: Synthetic polynucleotide
  source 1..111: mol_type = other DNA
tccacaaaatgattctgaattagctgtatcgtcaaggcactcttgcctacgccarcagctccaactaccacaagtttatattcagtcattttcagcaggccttataataaa

SEQ ID NO: 52 - DNA, 111 bp, synthetic construct
  misc_feature 1..111: note = Description of Artificial Sequence: Synthetic polynucleotide
  source 1..111: mol_type = other DNA
attataaggcctgctgaaaatgactgaatataaacttgtggtagttggagctggtgrcgtaggcaagagtgccttgacgatacagctaattcagaatcattttgtggacga

SEQ ID NO: 53 - DNA, 111 bp, synthetic construct
  misc_feature 1..111: note = Description of Artificial Sequence: Synthetic polynucleotide
  source 1..111: mol_type = other DNA
aacaagtggttatagatggtgaaacctgtttgttggacatactggatacagctggamaagaagagtacagtgccatgagagaccaatacatgaggacaggcgaaggcttcc

SEQ ID NO: 54 - DNA, 111 bp, synthetic construct
  misc_feature 1..111: note = Description of Artificial Sequence: Synthetic polynucleotide
  source 1..111: mol_type = other DNA
caagtggttatagatggtgaaacctgtttgttggacatactggatacagctggacawgaagagtacagtgccatgagagaccaatacatgaggacaggcgaaggcttcctc

SEQ ID NO: 55 - DNA, 111 bp, synthetic construct; identical to SEQ ID NO: 43
  misc_feature 1..111: note = Description of Artificial Sequence: Synthetic polynucleotide
  source 1..111: mol_type = other DNA

SEQ ID NO: 56 - DNA, 111 bp, synthetic construct; identical to SEQ ID NO: 44
  misc_feature 1..111: note = Description of Artificial Sequence: Synthetic polynucleotide
  variation 76: note = n is a, c, t, g, unknown or other
  variation 83: note = n is a, c, t, g, unknown or other
  source 1..111: mol_type = other DNA

SEQ ID NO: 57 - DNA, 111 bp, synthetic construct; identical to SEQ ID NO: 45
  misc_feature 1..111: note = Description of Artificial Sequence: Synthetic polynucleotide
  variation 89: note = n is a, c, t, g, unknown or other
  variation 96: note = n is a, c, t, g, unknown or other
  source 1..111: mol_type = other DNA

SEQ ID NO: 58 - DNA, 111 bp, synthetic construct; identical to SEQ ID NO: 46
  misc_feature 1..111: note = Description of Artificial Sequence: Synthetic polynucleotide
  source 1..111: mol_type = other DNA

SEQ ID NO: 59 - DNA, 111 bp, synthetic construct; identical to SEQ ID NO: 47
  misc_feature 1..111: note = Description of Artificial Sequence: Synthetic polynucleotide
  source 1..111: mol_type = other DNA

SEQ ID NO: 60 - DNA, 111 bp, synthetic construct; identical to SEQ ID NO: 48
  misc_feature 1..111: note = Description of Artificial Sequence: Synthetic polynucleotide
  variation 63: note = n is a, c, t, g, unknown or other
  source 1..111: mol_type = other DNA

SEQ ID NO: 61 - DNA, 111 bp, synthetic construct
  misc_feature 1..111: note = Description of Artificial Sequence: Synthetic polynucleotide
  source 1..111: mol_type = other DNA
aagtagtaattgatggagaaacctgtctcttggatattctcgacacagcaggtcasgaggagtacagtgcaatgagggaccagtacatgaggactggggagggctttcttt

SEQ ID NO: 62 - DNA, 111 bp, synthetic construct; identical to SEQ ID NO: 50
  misc_feature 1..111: note = Description of Artificial Sequence: Synthetic polynucleotide
  source 1..111: mol_type = other DNA

SEQ ID NO: 63 - DNA, 111 bp, synthetic construct; identical to SEQ ID NO: 51
  misc_feature 1..111: note = Description of Artificial Sequence: Synthetic polynucleotide
  source 1..111: mol_type = other DNA

SEQ ID NO: 64 - DNA, 111 bp, synthetic construct; identical to SEQ ID NO: 52
  misc_feature 1..111: note = Description of Artificial Sequence: Synthetic polynucleotide
  source 1..111: mol_type = other DNA

SEQ ID NO: 65 - DNA, 111 bp, synthetic construct; identical to SEQ ID NO: 53
  misc_feature 1..111: note = Description of Artificial Sequence: Synthetic polynucleotide
  source 1..111: mol_type = other DNA

SEQ ID NO: 66 - DNA, 111 bp, synthetic construct; identical to SEQ ID NO: 54
  misc_feature 1..111: note = Description of Artificial Sequence: Synthetic polynucleotide
  source 1..111: mol_type = other DNA

SEQ ID NO: 67 - DNA, 111 bp, synthetic construct; identical to SEQ ID NO: 43
  misc_feature 1..111: note = Description of Artificial Sequence: Synthetic polynucleotide
  source 1..111: mol_type = other DNA

SEQ ID NO: 68 - DNA, 111 bp, synthetic construct; identical to SEQ ID NO: 44
  misc_feature 1..111: note = Description of Artificial Sequence: Synthetic polynucleotide
  variation 76: note = n is a, c, t, g, unknown or other
  variation 83: note = n is a, c, t, g, unknown or other
  source 1..111: mol_type = other DNA

SEQ ID NO: 69 - DNA, 111 bp, synthetic construct; identical to SEQ ID NO: 45
  misc_feature 1..111: note = Description of Artificial Sequence: Synthetic polynucleotide
  variation 89: note = n is a, c, t, g, unknown or other
  variation 96: note = n is a, c, t, g, unknown or other
  source 1..111: mol_type = other DNA

SEQ ID NO: 70 - DNA, 111 bp, synthetic construct; identical to SEQ ID NO: 46
  misc_feature 1..111: note = Description of Artificial Sequence: Synthetic polynucleotide
  source 1..111: mol_type = other DNA

SEQ ID NO: 71 - DNA, 111 bp, synthetic construct
  misc_feature 1..111: note = Description of Artificial Sequence: Synthetic polynucleotide
  source 1..111: mol_type = other DNA
ttactttgcctccttctgcatggtattctttctcttccgcacccagcagtttggccygcccaaaatctgtgatcttgacatgctgcggtgttttcaccagtacgttcctgg

SEQ ID NO: 72 - DNA, 111 bp, synthetic construct
  misc_feature 1..111: note = Description of Artificial Sequence: Synthetic polynucleotide
  variation 63: note = n is a, c, t, g, unknown or other
  source 1..111: mol_type = other DNA
aggaagcaagtagtaattgatggagaaacctgtctcttggatattctcgacacagkaggtcangaggagtacagtgcaatgagggaccagtacatgaggactggggagggc

SEQ ID NO: 73 - DNA, 111 bp, synthetic construct; identical to SEQ ID NO: 49
  misc_feature 1..111: note = Description of Artificial Sequence: Synthetic polynucleotide
  source 1..111: mol_type = other DNA

SEQ ID NO: 74 - DNA, 111 bp, synthetic construct; identical to SEQ ID NO: 50
  misc_feature 1..111: note = Description of Artificial Sequence: Synthetic polynucleotide
  source 1..111: mol_type = other DNA

SEQ ID NO: 75 - DNA, 111 bp, synthetic construct; identical to SEQ ID NO: 51
  misc_feature 1..111: note = Description of Artificial Sequence: Synthetic polynucleotide
  source 1..111: mol_type = other DNA

SEQ ID NO: 76 - DNA, 111 bp, synthetic construct; identical to SEQ ID NO: 52
  misc_feature 1..111: note = Description of Artificial Sequence: Synthetic polynucleotide
  source 1..111: mol_type = other DNA

SEQ ID NO: 77 - DNA, 111 bp, synthetic construct; identical to SEQ ID NO: 53
  misc_feature 1..111: note = Description of Artificial Sequence: Synthetic polynucleotide
  source 1..111: mol_type = other DNA

SEQ ID NO: 78 - DNA, 111 bp, synthetic construct; identical to SEQ ID NO: 54
  misc_feature 1..111: note = Description of Artificial Sequence: Synthetic polynucleotide
  source 1..111: mol_type = other DNA

SEQ ID NO: 79 - DNA, 290 bp, Homo sapiens
  source 1..290: mol_type = genomic DNA
gtactggtggagtatttgatagtgtattaaccttatgtgtgacatgttctaatatagtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggaccattctttgatacagataaaggtttctctgaccattttcatgagt

SEQ ID NO: 80 - DNA, 16 bp, synthetic construct
  misc_feature 1..16: note = Description of Artificial Sequence: Synthetic primer
  source 1..16: mol_type = other DNA
cccgctgctcctgtgc

SEQ ID NO: 81 - DNA, 20 bp, synthetic construct
  misc_feature 1..20: note = Description of Artificial Sequence: Synthetic primer
  source 1..20: mol_type = other DNA
acccaacgcacaacttagcg

SEQ ID NO: 82 - DNA, 25 bp, synthetic construct
  misc_feature 1..25: note = Description of Artificial Sequence: Synthetic primer
  source 1..25: mol_type = other DNA
cccgccttgatggatggagatttac

SEQ ID NO: 83 - DNA, 24 bp, synthetic construct
  misc_feature 1..24: note = Description of Artificial Sequence: Synthetic primer
  source 1..24: mol_type = other DNA
gcacccttggtgaaacaattgttg

SEQ ID NO: 84 - DNA, 25 bp, synthetic construct
  misc_feature 1..25: note = Description of Artificial Sequence: Synthetic primer
  source 1..25: mol_type = other DNA
cgccgaactggaagtattttgacag

SEQ ID NO: 85 - DNA, 23 bp, synthetic construct
  misc_feature 1..23: note = Description of Artificial Sequence: Synthetic primer
  source 1..23: mol_type = other DNA
tatccctcacagagagaagggag

SEQ ID NO: 86 - DNA, 19 bp, synthetic construct
  misc_feature 1..19: note = Description of Artificial Sequence: Synthetic primer
  source 1..19: mol_type = other DNA
gcttgcaatggctccaacc

SEQ ID NO: 87 - DNA, 19 bp, synthetic construct
  misc_feature 1..19: note = Description of Artificial Sequence: Synthetic primer
  source 1..19: mol_type = other DNA
ggagccattgcaagccaag

SEQ ID NO: 88 - DNA, 21 bp, synthetic construct
  misc_feature 1..21: note = Description of Artificial Sequence: Synthetic primer
  source 1..21: mol_type = other DNA
tggcaagagcgatgagtactc

SEQ ID NO: 89 - DNA, 25 bp, synthetic construct
  misc_feature 1..25: note = Description of Artificial Sequence: Synthetic primer
  source 1..25: mol_type = other DNA
ctctcaaaaggtaactgcccactta

SEQ ID NO: 90 - DNA, 16 bp, synthetic construct
  misc_feature 1..16: note = Description of Artificial Sequence: Synthetic probe
  source 1..16: mol_type = other DNA
ctcctgtgctgtcacc

SEQ ID NO: 91 - DNA, 15 bp, synthetic construct
  misc_feature 1..15: note = Description of Artificial Sequence: Synthetic probe
  source 1..15: mol_type = other DNA
cttagcggcacgcac

SEQ ID NO: 92 - DNA, 18 bp, synthetic construct
  misc_feature 1..18: note = Description of Artificial Sequence: Synthetic probe
  source 1..18: mol_type = other DNA
ggagatttacgcaatgtg

SEQ ID NO: 93 - DNA, 17 bp, synthetic construct
  misc_feature 1..17: note = Description of Artificial Sequence: Synthetic probe
  source 1..17: mol_type = other DNA
acaattgttgagggggg

SEQ ID NO: 94 - DNA, 20 bp, synthetic construct
  misc_feature 1..20: note = Description of Artificial Sequence: Synthetic probe
  source 1..20: mol_type = other DNA
aagtattttgacagctttac

SEQ ID NO: 95 - DNA, 17 bp, synthetic construct
  misc_feature 1..17: note = Description of Artificial Sequence: Synthetic primer
  source 1..17: mol_type = other DNA
cccggctgctcctgtgt

SEQ ID NO: 96 - DNA, 20 bp, synthetic construct
  misc_feature 1..20: note = Description of Artificial Sequence: Synthetic primer
  source 1..20: mol_type = other DNA
gccgaacgcacaacttagca

SEQ ID NO: 97 - DNA, 25 bp, synthetic construct
  misc_feature 1..25: note = Description of Artificial Sequence: Synthetic primer
  source 1..25: mol_type = other DNA
cccgccttgatggatggagatttat

SEQ ID NO: 98 - DNA, 25 bp, synthetic construct
  misc_feature 1..25: note = Description of Artificial Sequence: Synthetic primer
  source 1..25: mol_type = other DNA
tgcacccttggtgaaacaattgttc

SEQ ID NO: 99 - DNA, 25 bp, synthetic construct
  misc_feature 1..25: note = Description of Artificial Sequence: Synthetic primer
  source 1..25: mol_type = other DNA
cgccgaactggaagtattttgacat

SEQ ID NO: 100 - DNA, 19 bp, synthetic construct
  misc_feature 1..19: note = Description of Artificial Sequence: Synthetic probe
  source 1..19: mol_type = other DNA
atggccctgataagggaga

SEQ ID NO: 101 - DNA, 15 bp, synthetic construct
  misc_feature 1..15: note = Description of Artificial Sequence: Synthetic probe
  source 1..15: mol_type = other DNA
agggcggtgctcgag

SEQ ID NO: 102 - DNA, 19 bp, synthetic construct
  misc_feature 1..19: note = Description of Artificial Sequence: Synthetic probe
  source 1..19: mol_type = other DNA
tgtcactctacccagtaaa

SEQ ID NO: 103 - DNA, 21 bp, synthetic construct
  misc_feature 1..21: note = Description of Artificial Sequence: Synthetic probe
  source 1..21: mol_type = other DNA
agaatctgccaattacctaga

SEQ ID NO: 104 - DNA, 21 bp, synthetic construct
  misc_feature 1..21: note = Description of Artificial Sequence: Synthetic probe
  source 1..21: mol_type = other DNA
acaagtagctgataataaatg

SEQ ID NO: 105 - DNA, 17 bp, synthetic construct
  misc_feature 1..17: note = Description of Artificial Sequence: Synthetic probe
  source 1..17: mol_type = other DNA
gctcctgtgttgtcacc

SEQ ID NO: 106 - DNA, 16 bp, synthetic construct
  misc_feature 1..16: note = Description of Artificial Sequence: Synthetic probe
  source 1..16: mol_type = other DNA
cttagcagcacgcacc

SEQ ID NO: 107 - DNA, 19 bp, synthetic construct
  misc_feature 1..19: note = Description of Artificial Sequence: Synthetic probe
  source 1..19: mol_type = other DNA
tggagatttatgcaatgtg

SEQ ID NO: 108 - DNA, 17 bp, synthetic construct
  misc_feature 1..17: note = Description of Artificial Sequence: Synthetic probe
  source 1..17: mol_type = other DNA
acaattgttcagggggg

SEQ ID NO: 109 - DNA, 20 bp, synthetic construct
  misc_feature 1..20: note = Description of Artificial Sequence: Synthetic probe
  source 1..20: mol_type = other DNA
aagtattttgacatctttac

SEQ ID NO: 110 - DNA, 24 bp, synthetic construct
  misc_feature 1..24: note = Description of Artificial Sequence: Synthetic primer
  source 1..24: mol_type = other DNA
gcctgattttggtctagctacaga

SEQ ID NO: 111 - DNA, 19 bp, synthetic construct
  misc_feature 1..19: note = Description of Artificial Sequence: Synthetic primer
  source 1..19: mol_type = other DNA
cgcgagaaggagctgtggt

SEQ ID NO: 112 - DNA, 21 bp, synthetic construct
  misc_feature 1..21: note = Description of Artificial Sequence: Synthetic primer
  source 1..21: mol_type = other DNA
ccgtgcctttaccactcagag

SEQ ID NO: 113 - DNA, 18 bp, synthetic construct
  misc_feature 1..18: note = Description of Artificial Sequence: Synthetic primer
  source 1..18: mol_type = other DNA
gcgcgtgcagctcatcac

SEQ ID NO: 114 - DNA, 17 bp, synthetic construct
  misc_feature 1..17: note = Description of Artificial Sequence: Synthetic primer
  source 1..17: mol_type = other DNA
cggcagcagtttggccc

SEQ ID NO: 115 - DNA, 21 bp, synthetic construct
  misc_feature 1..21: note = Description of Artificial Sequence: Synthetic primer
  source 1..21: mol_type = other DNA
gccggatattctcgacacagc

SEQ ID NO: 116 - DNA, 18 bp, synthetic construct
  misc_feature 1..18: note = Description of Artificial Sequence: Synthetic primer
  source 1..18: mol_type = other DNA
gcggacacagcaggtcaa

SEQ ID NO: 117 - DNA, 18 bp, synthetic construct
  misc_feature 1..18: note = Description of Artificial Sequence: Synthetic primer
  source 1..18: mol_type = other DNA
gcggacacagcaggtcaa

SEQ ID NO: 118 - DNA, 18 bp, synthetic construct
  misc_feature 1..18: note = Description of Artificial Sequence: Synthetic primer
  source 1..18: mol_type = other DNA
cgccttgcctacgccact

SEQ ID NO: 119 - DNA, 16 bp, synthetic construct
  misc_feature 1..16: note = Description of Artificial Sequence: Synthetic primer
  source 1..16: mol_type = other DNA
cgctgcctacgccacg

SEQ ID NO: 120 - DNA, 17 bp, synthetic construct
  misc_feature 1..17: note = Description of Artificial Sequence: Synthetic primer
  source 1..17: mol_type = other DNA
gcgttgcctacgccacc

SEQ ID NO: 121 - DNA, 19 bp, synthetic construct
  misc_feature 1..19: note = Description of Artificial Sequence: Synthetic primer
  source 1..19: mol_type = other DNA
cgcctcttgcctacgccat

SEQ ID NO: 122 - DNA, 18 bp, synthetic construct
  misc_feature 1..18: note = Description of Artificial Sequence: Synthetic primer
  source 1..18: mol_type = other DNA
gcgtcttgcctacgccag

SEQ ID NO: 123 - DNA, 18 bp, synthetic construct
  misc_feature 1..18: note = Description of Artificial Sequence: Synthetic primer
  source 1..18: mol_type = other DNA
gcgtcttgcctacgccac

SEQ ID NO: 124 - DNA, 20 bp, synthetic construct
  misc_feature 1..20: note = Description of Artificial Sequence: Synthetic primer
  source 1..20: mol_type = other DNA
cgcgtagttggagctggtga

SEQ ID NO: 125 - DNA, 22 bp, synthetic construct
  misc_feature 1..22: note = Description of Artificial Sequence: Synthetic primer
  source 1..22: mol_type = other DNA
ccgatactggatacagctggaa

SEQ ID NO: 126 - DNA, 20 bp, synthetic construct
  misc_feature 1..20: note = Description of Artificial Sequence: Synthetic primer
  source 1..20: mol_type = other DNA
ccgtggatacagctggacaa

SEQ ID NO: 127 - DNA, 18 bp, synthetic construct
  misc_feature 1..18: note = Description of Artificial Sequence: Synthetic primer
  source 1..18: mol_type = other DNA
ccgggtggttggagcaga

SEQ ID NO: 128 - DNA, 18 bp, synthetic construct
  misc_feature 1..18: note = Description of Artificial Sequence: Synthetic primer
  source 1..18: mol_type = other DNA
ccgggttggagcaggtga

SEQ ID NO: 129 - DNA, 18 bp, synthetic construct
  misc_feature 1..18: note = Description of Artificial Sequence: Synthetic primer
  source 1..18: mol_type = other DNA
ccgggaggttgtgaggca

SEQ ID NO: 130 - DNA, 23 bp, synthetic construct
  misc_feature 1..23: note = Description of Artificial Sequence: Synthetic primer
  source 1..23: mol_type = other DNA
gccggatgacagaaacacttttc

SEQ ID NO: 131 - DNA, 25 bp, synthetic construct
  misc_feature 1..25: note = Description of Artificial Sequence: Synthetic primer
  source 1..25: mol_type = other DNA
gcgtacaactacatgtgtaacagtg

SEQ ID NO: 132 - DNA, 17 bp, synthetic construct
  misc_feature 1..17: note = Description of Artificial Sequence: Synthetic primer
  source 1..17: mol_type = other DNA
gccttcctgcatgggca

SEQ ID NO: 133 - DNA, 16 bp, synthetic construct
  misc_feature 1..16: note = Description of Artificial Sequence: Synthetic primer
  source 1..16: mol_type = other DNA
gccggcggcatgaacc

SEQ ID NO: 134 - DNA, 16 bp, synthetic construct
  misc_feature 1..16: note = Description of Artificial Sequence: Synthetic primer
  source 1..16: mol_type = other DNA
gccgcggcatgaacca

SEQ ID NO: 135 - DNA, 22 bp, synthetic construct
  misc_feature 1..22: note = Description of Artificial Sequence: Synthetic primer
  source 1..22: mol_type = other DNA
gccgccaacagctttgaggtgc

SEQ ID NO: 136 - DNA, 23 bp, synthetic construct
  misc_feature 1..23: note = Description of Artificial Sequence: Synthetic primer
  source 1..23: mol_type = other DNA
ccggattttggtctagctacagt

SEQ ID NO: 137 - DNA, 18 bp, synthetic construct
  misc_feature 1..18: note = Description of Artificial Sequence: Synthetic primer
  source 1..18: mol_type = other DNA
gccagaaggagctgtggc

SEQ ID NO: 138 - DNA, 21 bp, synthetic construct
  misc_feature 1..21: note = Description of Artificial Sequence: Synthetic primer
  source 1..21: mol_type = other DNA
ccgtgcctttaccactcagaa

SEQ ID NO: 139 - DNA, 18 bp, synthetic construct
  misc_feature 1..18: note = Description of Artificial Sequence: Synthetic primer
  source 1..18: mol_type = other DNA
gcgcgtgcagctcatcat

SEQ ID NO: 140 - DNA, 17 bp, synthetic construct
  misc_feature 1..17: note = Description of Artificial Sequence: Synthetic primer
  source 1..17: mol_type = other DNA
cggcagcagtttggcca

SEQ ID NO: 141 - DNA, 22 bp, synthetic construct
  misc_feature 1..22: note = Description of Artificial Sequence: Synthetic primer
  source 1..22: mol_type = other DNA
cgctggatattctcgacacagg

SEQ ID NO: 142 - DNA, 18 bp, synthetic construct
  misc_feature 1..18: note = Description of Artificial Sequence: Synthetic primer
  source 1..18: mol_type = other DNA
gcggacacagcaggtcac

SEQ ID NO: 143 - DNA, 18 bp, synthetic construct
  misc_feature 1..18: note = Description of Artificial Sequence: Synthetic primer
  source 1..18: mol_type = other DNA
gcggacacagcaggtcat

SEQ ID NO: 144 - DNA, 17 bp, synthetic construct
  misc_feature 1..17: note = Description of Artificial Sequence: Synthetic primer
  source 1..17: mol_type = other DNA
gcgttgcctacgccacc

SEQ ID NO: 145 - DNA, 17 bp, synthetic construct
  misc_feature 1..17: note = Description of Artificial Sequence: Synthetic primer
  source 1..17: mol_type = other DNA
gcgttgcctacgccacc

SEQ ID NO: 146 - DNA, 17 bp, synthetic construct
  misc_feature 1..17: note = Description of Artificial Sequence: Synthetic primer
  source 1..17: mol_type = other DNA
gcgttgcctacgccaca

SEQ ID NO: 147 - DNA, 18 bp, synthetic construct
  misc_feature 1..18: note = Description of Artificial Sequence: Synthetic primer
  source 1..18: mol_type = other DNA
gcgtcttgcctacgccac

SEQ ID NO: 148 - DNA, 18 bp, synthetic construct
  misc_feature 1..18: note = Description of Artificial Sequence: Synthetic primer
  source 1..18: mol_type = other DNA
gcgtcttgcctacgccac

SEQ ID NO: 149 - DNA, 18 bp, synthetic construct
  misc_feature 1..18: note = Description of Artificial Sequence: Synthetic primer
  source 1..18: mol_type = other DNA
gcgtcttgcctacgccaa

SEQ ID NO: 150 - DNA, 19 bp, synthetic construct
  misc_feature 1..19: note = Description of Artificial Sequence: Synthetic primer
  source 1..19: mol_type = other DNA
gcctagttggagctggtgg

SEQ ID NO: 151 - DNA, 22 bp, synthetic construct
  misc_feature 1..22: note = Description of Artificial Sequence: Synthetic primer
  source 1..22: mol_type = other DNA
ccgatactggatacagctggac

SEQ ID NO: 152 - DNA, 21 bp, synthetic construct
  misc_feature 1..21: note = Description of Artificial Sequence: Synthetic primer
  source 1..21: mol_type = other DNA
cgcctggatacagctggacat

SEQ ID NO: 153 - DNA, 17 bp, synthetic construct
  misc_feature 1..17: note = Description of Artificial Sequence: Synthetic primer
  source 1..17: mol_type = other DNA
cgcgtggttggagcagg

SEQ ID NO: 154 - DNA, 17 bp, synthetic construct
  misc_feature 1..17: note = Description of Artificial Sequence: Synthetic primer
  source 1..17: mol_type = other DNA
cgcgttggagcaggtgg

SEQ ID NO: 155 - DNA, 17 bp, synthetic construct
  misc_feature 1..17: note = Description of Artificial Sequence: Synthetic primer
  source 1..17: mol_type = other DNA
ggcgaggttgtgaggcg

SEQ ID NO: 156 - DNA, 24 bp, synthetic construct
  misc_feature 1..24: note = Description of Artificial Sequence: Synthetic primer
  source 1..24: mol_type = other DNA
gcctggatgacagaaacacttttt

SEQ ID NO: 157 - DNA, 26 bp, synthetic construct
  misc_feature 1..26: note = Description of Artificial Sequence: Synthetic primer
  source 1..26: mol_type = other DNA
ggcctacaactacatgtgtaacagtt

SEQ ID NO: 158 - DNA, 16 bp, synthetic construct
  misc_feature 1..16: note = Description of Artificial Sequence: Synthetic primer
  source 1..16: mol_type = other DNA
gcctcctgcatgggcg

SEQ ID NO: 159 - DNA, 16 bp, synthetic construct
  misc_feature 1..16: note = Description of Artificial Sequence: Synthetic primer
  source 1..16: mol_type = other DNA
gccggcggcatgaact

SEQ ID NO: 160 - DNA, 16 bp, synthetic construct
  misc_feature 1..16: note = Description of Artificial Sequence: Synthetic primer
  source 1..16: mol_type = other DNA
gccgcggcatgaaccg

SEQ ID NO: 161 - DNA, 20 bp, synthetic construct
  misc_feature 1..20: note = Description of Artificial Sequence: Synthetic primer
  source 1..20: mol_type = other DNA
cgcgaacagctttgaggtgt

SEQ ID NO: 162 - DNA, 19 bp, synthetic construct
  misc_feature 1..19: note = Description of Artificial Sequence: Synthetic primer
  source 1..19: mol_type = other DNA
cccgtggtctagctacaga

SEQ ID NO: 163 - DNA, 17 bp, synthetic construct
  misc_feature 1..17: note = Description of Artificial Sequence: Synthetic primer
  source 1..17: mol_type = other DNA
ccccaaggagctgtggt

SEQ ID NO: 164 - DNA, 20 bp, synthetic construct
  misc_feature 1..20: note = Description of Artificial Sequence: Synthetic primer
  source 1..20: mol_type = other DNA
cgcccctttaccactcagag

SEQ ID NO: 165 - DNA, 18 bp, synthetic construct
  misc_feature 1..18: note = Description of Artificial Sequence: Synthetic primer
  source 1..18: mol_type = other DNA
ggggatgcagctcatcac

SEQ ID NO: 166 - DNA, 15 bp, synthetic construct
  misc_feature 1..15: note = Description of Artificial Sequence: Synthetic primer
  source 1..15: mol_type = other DNA
ggcgcagtttggccc

SEQ ID NO: 167 - DNA, 18 bp, synthetic construct
  misc_feature 1..18: note = Description of Artificial Sequence: Synthetic primer
  source 1..18: mol_type = other DNA
cccgattctcgacacagc

SEQ ID NO: 168 - DNA, 17 bp, synthetic construct
  misc_feature 1..17: note = Description of Artificial Sequence: Synthetic primer
  source 1..17: mol_type = other DNA
cgcccacagcaggtcaa

SEQ ID NO: 169 - DNA, 17 bp, synthetic construct
  misc_feature 1..17: note = Description of Artificial Sequence: Synthetic primer
  source 1..17: mol_type = other DNA
tgcccacagcaggtcaa

SEQ ID NO: 170 - DNA, 15 bp, synthetic construct
  misc_feature 1..15: note = Description of Artificial Sequence: Synthetic primer
  source 1..15: mol_type = other DNA
gccgcctacgccact

SEQ ID NO: 171 - DNA, 15 bp, synthetic construct
  misc_feature 1..15: note = Description of Artificial Sequence: Synthetic primer
  source 1..15: mol_type = other DNA
agcccctacgccacg

SEQ ID NO: 172 - DNA, 15 bp, synthetic construct
  misc_feature 1..15: note = Description of Artificial Sequence: Synthetic primer
  source 1..15: mol_type = other DNA
accccctacgccacc

SEQ ID NO: 173 - DNA, 15 bp, synthetic construct
  misc_feature 1..15: note = Description of Artificial Sequence: Synthetic primer
  source 1..15: mol_type = other DNA
ggctgcctacgccat

SEQ ID NO: 174 - DNA, 16 bp, synthetic construct
  misc_feature 1..16: note = Description of Artificial Sequence: Synthetic primer
  source 1..16: mol_type = other DNA
gggctgcctacgccag

SEQ ID NO: 175 - DNA, 15 bp, synthetic construct
  misc_feature 1..15: note = Description of Artificial Sequence: Synthetic primer
  source 1..15: mol_type = other DNA
ggctgcctacgccac

SEQ ID NO: 176 - DNA, 18 bp, synthetic construct
  misc_feature 1..18: note = Description of Artificial Sequence: Synthetic primer
  source 1..18: mol_type = other DNA
ccccgttggagctggtga

SEQ ID NO: 177 - DNA, 19 bp, synthetic construct
  misc_feature 1..19: note = Description of Artificial Sequence: Synthetic primer
  source 1..19: mol_type = other DNA
cgggtggatacagctggaa

SEQ ID NO: 178 - DNA, 19 bp, synthetic construct
  misc_feature 1..19: note = Description of Artificial Sequence: Synthetic primer
  source 1..19: mol_type = other DNA
gggcgatacagctggacaa

SEQ ID NO: 179 - DNA, 17 bp, synthetic construct
  misc_feature 1..17: note = Description of Artificial Sequence: Synthetic primer
  source 1..17: mol_type = other DNA
gccctggttggagcaga

SEQ ID NO: 180 - DNA, 17 bp, synthetic construct
  misc_feature 1..17: note = Description of Artificial Sequence: Synthetic primer
  source 1..17: mol_type = other DNA
cccgttggagcaggtga

SEQ ID NO: 181 - DNA, 16 bp, synthetic construct
  misc_feature 1..16: note = Description of Artificial Sequence: Synthetic primer
  source 1..16: mol_type = other DNA
gccaggttgtgaggca

SEQ ID NO: 182 - DNA, 22 bp, synthetic construct
  misc_feature 1..22: note = Description of Artificial Sequence: Synthetic primer
  source 1..22: mol_type = other DNA
tgccctgacagaaacacttttc

SEQ ID NO: 183 - DNA, 21 bp, synthetic construct
  misc_feature 1..21: note = Description of Artificial Sequence: Synthetic primer
  source 1..21: mol_type = other DNA
cgccctacatgtgtaacagtg

SEQ ID NO: 184 - DNA, 15 bp, synthetic construct
  misc_feature 1..15: note = Description of Artificial Sequence: Synthetic primer
  source 1..15: mol_type = other DNA
ccgcctgcatgggca

SEQ ID NO: 185 - DNA, 15 bp, synthetic construct
  misc_feature 1..15: note = Description of Artificial Sequence: Synthetic primer
  source 1..15: mol_type = other DNA
cccgcggcatgaacc

SEQ ID NO: 186 - DNA, 15 bp, synthetic construct
  misc_feature 1..15: note = Description of Artificial Sequence: Synthetic primer
  source 1..15: mol_type = other DNA
ccccggcatgaacca

SEQ ID NO: 187 - DNA, 17 bp, synthetic construct
  misc_feature 1..17: note = Description of Artificial Sequence: Synthetic primer
  source 1..17: mol_type = other DNA
cgccagctttgaggtgc

SEQ ID NO: 188 - DNA, 19 bp, synthetic construct
  misc_feature 1..19: note = Description of Artificial Sequence: Synthetic primer
  source 1..19: mol_type = other DNA
cccgtggtctagctacagt

SEQ ID NO: 189 - DNA, 16 bp, synthetic construct
  misc_feature 1..16: note = Description of Artificial Sequence: Synthetic primer
  source 1..16: mol_type = other DNA
ccccaggagctgtggc

SEQ ID NO: 190 - DNA, 20 bp, synthetic construct
  misc_feature 1..20: note = Description of Artificial Sequence: Synthetic primer
  source 1..20: mol_type = other DNA
cgcccctttaccactcagaa

SEQ ID NO: 191 - DNA, 19 bp, synthetic construct
  misc_feature 1..19: note = Description of Artificial Sequence: Synthetic primer
  source 1..19: mol_type = other DNA
ggggagtgcagctcatcat

SEQ ID NO: 192 - DNA, 15 bp, synthetic construct
  misc_feature 1..15: note = Description of Artificial Sequence: Synthetic primer
  source 1..15: mol_type = other DNA
ggcgcagtttggcca

SEQ ID NO: 193 - DNA, 18 bp, synthetic construct
  misc_feature 1..18: note = Description of Artificial Sequence: Synthetic primer
  source 1..18: mol_type = other DNA
cccgattctcgacacagg

SEQ ID NO: 194 - DNA, 17 bp, synthetic construct
  misc_feature 1..17: note = Description of Artificial Sequence: Synthetic primer
  source 1..17: mol_type = other DNA
tgcccacagcaggtcac

SEQ ID NO: 195 - DNA, 17 bp, synthetic construct
  misc_feature 1..17: note = Description of Artificial Sequence: Synthetic primer
  source 1..17: mol_type = other DNA
cgcccacagcaggtcat

SEQ ID NO: 196 - DNA, 14 bp, synthetic construct
  misc_feature 1..14: note = Description of Artificial Sequence: Synthetic primer
  source 1..14: mol_type = other DNA
gcccctacgccacc

SEQ ID NO: 197 - DNA, 15 bp, synthetic construct
  misc_feature 1..15: note = Description of Artificial Sequence: Synthetic primer
  source 1..15: mol_type = other DNA
agcccctacgccacc

SEQ ID NO: 198 - DNA, 15 bp, synthetic construct
  misc_feature 1..15: note = Description of Artificial Sequence: Synthetic primer
  source 1..15: mol_type = other DNA
accgcctacgccaca

SEQ ID NO: 199 - DNA, 16 bp, synthetic construct
  misc_feature 1..16: note = Description of Artificial Sequence: Synthetic primer
  source 1..16: mol_type = other DNA
gggctgcctacgccac

SEQ ID NO: 200 - DNA, 15 bp, synthetic construct
  misc_feature 1..15: note = Description of Artificial Sequence: Synthetic primer
  source 1..15: mol_type = other DNA
ggctgcctacgccac

SEQ ID NO: 201 - DNA, 16 bp, synthetic construct
  misc_feature 1..16: note = Description of Artificial Sequence: Synthetic primer
  source 1..16: mol_type = other DNA
gggctgcctacgccaa

SEQ ID NO: 202 - DNA, 17 bp, synthetic construct
  misc_feature 1..17: note = Description of Artificial Sequence: Synthetic primer
  source 1..17: mol_type = other DNA
ccccttggagctggtgg

SEQ ID NO: 203 - DNA, 18 bp, synthetic construct
  misc_feature 1..18: note = Description of Artificial Sequence: Synthetic primer
  source 1..18: mol_type = other DNA
cgggggatacagctggac

SEQ ID NO: 204 - DNA, 19 bp, synthetic construct
  misc_feature 1..19: note = Description of Artificial Sequence: Synthetic primer
  source 1..19: mol_type = other DNA
gggcgatacagctggacat

SEQ ID NO: 205 - DNA, 16 bp, synthetic construct
  misc_feature 1..16: note = Description of Artificial Sequence: Synthetic primer
  source 1..16: mol_type = other DNA
gcccggttggagcagg

SEQ ID NO: 206 - DNA, 16 bp, synthetic construct
  misc_feature 1..16: note = Description of Artificial Sequence: Synthetic primer
  source 1..16: mol_type = other DNA
cccttggagcaggtgg

SEQ ID NO: 207 - DNA, 16 bp, synthetic construct
  misc_feature 1..16: note = Description of Artificial Sequence: Synthetic primer
  source 1..16: mol_type = other DNA
gccaggttgtgaggcg

SEQ ID NO: 208 - DNA, 22 bp, synthetic construct
  misc_feature 1..22: note = Description of Artificial Sequence: Synthetic primer
  source 1..22: mol_type = other DNA
tgccctgacagaaacacttttt

SEQ ID NO: 209 - DNA, 21 bp, synthetic construct
  misc_feature 1..21: note = Description of Artificial Sequence: Synthetic primer
  source 1..21: mol_type = other DNA
cgccctacatgtgtaacagtt

SEQ ID NO: 210 - DNA, 14 bp, synthetic construct
  misc_feature 1..14: note = Description of Artificial Sequence: Synthetic primer
  source 1..14: mol_type = other DNA
ccgctgcatgggcg

SEQ ID NO: 211 - DNA, 15 bp, synthetic construct
  misc_feature 1..15: note = Description of Artificial Sequence: Synthetic primer
  source 1..15: mol_type = other DNA
cccgcggcatgaact

SEQ ID NO: 212 - DNA, 15 bp, synthetic construct
  misc_feature 1..15: note = Description of Artificial Sequence: Synthetic primer
  source 1..15: mol_type = other DNA
ccccggcatgaaccg

SEQ ID NO: 213 - DNA, 17 bp, synthetic construct
  misc_feature 1..17: note = Description of Artificial Sequence: Synthetic primer
  source 1..17: mol_type = other DNA
cgccagctttgaggtgt

SEQ ID NO: 214 - DNA, 26 bp, synthetic construct
  misc_feature 1..26: note = Description of Artificial Sequence: Synthetic primer
  source 1..26: mol_type = other DNA
agcctcaattcttaccatccacaaaa

SEQ ID NO: 215 - DNA, 22 bp, synthetic construct
  misc_feature 1..22: note = Description of Artificial Sequence: Synthetic primer
  source 1..22: mol_type = other DNA
catggaaccagacagaaaagcg

SEQ ID NO: 216 - DNA, 22 bp, synthetic construct
  misc_feature 1..22: note = Description of Artificial Sequence: Synthetic primer
  source 1..22: mol_type = other DNA
gtcactggcagcaacagtctta

SEQ ID NO: 217 - DNA, 25 bp, synthetic construct
  misc_feature 1..25: note = Description of Artificial Sequence: Synthetic primer
  source 1..25: mol_type = other DNA
tgggagccaatattgtctttgtgtt

SEQ ID NO: 218 - DNA, 23 bp, synthetic construct
  misc_feature 1..23: note = Description of Artificial Sequence: Synthetic primer
  source 1..23: mol_type = other DNA
aggaacgtactggtgaaaacacc

SEQ ID NO: 219 - DNA, 20 bp, synthetic construct
  misc_feature 1..20: note = Description of Artificial Sequence: Synthetic primer
  source 1..20: mol_type = other DNA
ccctccccagtcctcatgta

SEQ ID NO: 220 - DNA, 41 bp, synthetic construct
  misc_feature 1..41: note = Description of Artificial Sequence: Synthetic primer
  source 1..41: mol_type = other DNA
tcttcaaatgatttagtattatttatggcaaatacacaaag

SEQ ID NO: 221 - DNA, 41 bp, synthetic construct; identical to SEQ ID NO: 220
  misc_feature 1..41: note = Description of Artificial Sequence: Synthetic primer
  source 1..41: mol_type = other DNA

SEQ ID NO: 222 - DNA, 28 bp, synthetic construct
  misc_feature 1..28: note = Description of Artificial Sequence: Synthetic primer
  source 1..28: mol_type = other DNA
tgtgtgacatgttctaatatagtcacat

SEQ ID NO: 223 - DNA, 28 bp, synthetic construct
  misc_feature 1..28: note = Description of Artificial Sequence: Synthetic primer
  source 1..28: mol_type = other DNA
tgtgtgacatgttctaatatagtcacat

SEQ ID NO: 224 - DNA, 28 bp, synthetic construct
  misc_feature 1..28: note = Description of Artificial Sequence: Synthetic primer
  source 1..28: mol_type = other DNA
tgtgtgacatgttctaatatagtcacat

SEQ ID NO: 225 - DNA, 28 bp, synthetic construct
  misc_feature 1..28: note = Description of Artificial Sequence: Synthetic primer
  source 1..28: mol_type = other DNA
tgtgtgacatgttctaatatagtcacat

SEQ ID NO: 226 - DNA, 28 bp, synthetic construct
  misc_feature 1..28: note = Description of Artificial Sequence: Synthetic primer
  source 1..28: mol_type = other DNA
tgtgtgacatgttctaatatagtcacat

SEQ ID NO: 227 - DNA, 28 bp, synthetic construct
  misc_feature 1..28: note = Description of Artificial Sequence: Synthetic primer
  source 1..28: mol_type = other DNA
tgtgtgacatgttctaatatagtcacat

SEQ ID NO: 228 - DNA, 22 bp, synthetic construct
  misc_feature 1..22: note = Description of Artificial Sequence: Synthetic primer
  source 1..22: mol_type = other DNA
ggtcctgcaccagtaatatgca

SEQ ID NO: 229 - DNA, 29 bp, synthetic construct
  misc_feature 1..29: note = Description of Artificial Sequence: Synthetic primer
  source 1..29: mol_type = other DNA
gcaaatgacttgctattattgatggcaaa

SEQ ID NO: 230 - DNA, 29 bp, synthetic construct
  misc_feature 1..29: note = Description of Artificial Sequence: Synthetic primer
  source 1..29: mol_type = other DNA
gcaaatgacttgctattattgatggcaaa

SEQ ID NO: 231 - DNA, 24 bp, synthetic construct
  misc_feature 1..24: note = Description of Artificial Sequence: Synthetic primer
  source 1..24: mol_type = other DNA
agtggttctggattagctggattg

SEQ ID NO: 232 - DNA, 24 bp, synthetic construct
  misc_feature 1..24: note = Description of Artificial Sequence: Synthetic primer
  source 1..24: mol_type = other DNA
gtggttctggattagctggattgt

SEQ ID NO: 233 - DNA, 18 bp, synthetic construct
  misc_feature 1..18: note = Description of Artificial Sequence: Synthetic primer
  source 1..18: mol_type = other DNA
gcaaccagccctgtcgtc

SEQ ID NO: 234 - DNA, 20 bp, synthetic construct
  misc_feature 1..20: note = Description of Artificial Sequence: Synthetic primer
  source 1..20: mol_type = other DNA
agaccccagttgcaaaccag

SEQ ID NO: 235 - DNA, 24 bp, synthetic construct
  misc_feature 1..24: note = Description of Artificial Sequence: Synthetic primer
  source 1..24: mol_type = other DNA
ctggagtcttccagtgtgatgatg

SEQ ID NO: 236 - DNA, 23 bp, synthetic construct
  misc_feature 1..23: note = Description of Artificial Sequence: Synthetic primer
  source 1..23: mol_type = other DNA
ctggagtcttccagtgtgatgat

SEQ ID NO: 237 - DNA, 17 bp, synthetic construct
  misc_feature 1..17: note = Description of Artificial Sequence: Synthetic primer
  source 1..17: mol_type = other DNA
tgtgcagggtggcaagt

SEQ ID NO: 238 - DNA, 17 bp, synthetic construct
  misc_feature 1..17: note = Description of Artificial Sequence: Synthetic primer
  source 1..17: mol_type = other DNA
tgtgcagggtggcaagt

SEQ ID NO: 239 - DNA, 24 bp, synthetic construct
  misc_feature 1..24: note = Description of Artificial Sequence: Synthetic primer
  source 1..24: mol_type = other DNA
ctttcttgcggagattctcttcct

SEQ ID NO: 240 - DNA, 19 bp, synthetic construct
  misc_feature 1..19: note = Description of Artificial Sequence: Synthetic probe
  source 1..19: mol_type = other DNA
cagacaactgttcaaactg

SEQ ID NO: 241 - DNA, 16 bp, synthetic construct
  misc_feature 1..16: note = Description of Artificial Sequence: Synthetic probe
  source 1..16: mol_type = other DNA
ctggcagcaacagtct

SEQ ID NO: 242 - DNA, 17 bp, synthetic construct
  misc_feature 1..17: note = Description of Artificial Sequence: Synthetic probe
  source 1..17: mol_type = other DNA
tagtggcaccagaatgg

SEQ ID NO: 243 - DNA, 16 bp, synthetic construct
  misc_feature 1..16: note = Description of Artificial Sequence: Synthetic probe
  source 1..16: mol_type = other DNA
cccttcggctgcctcc

SEQ ID NO: 244 - DNA, 17 bp, synthetic construct
  misc_feature 1..17: note = Description of Artificial Sequence: Synthetic probe
  source 1..17: mol_type = other DNA
cagcatgtcaagatcac

SEQ ID NO: 245 - DNA, 17 bp, synthetic construct
  misc_feature 1..17: note = Description of Artificial Sequence: Synthetic probe
  source 1..17: mol_type = other DNA
ctggtccctcattgcac

SEQ ID NO: 246 - DNA, 16 bp, synthetic construct
  misc_feature 1..16: note = Description of Artificial Sequence: Synthetic probe
  source 1..16: mol_type = other DNA
ccctccccagtcctca

SEQ ID NO: 247 - DNA, 16 bp, synthetic construct
  misc_feature 1..16: note = Description of Artificial Sequence: Synthetic probe
  source 1..16: mol_type = other DNA
ccctccccagtcctca

SEQ ID NO: 248 - DNA, 15 bp, synthetic construct
  misc_feature 1..15: note = Description of Artificial Sequence: Synthetic probe
  source 1..15: mol_type = other DNA
cctgctgaaaatgac

SEQ ID NO: 249 - DNA, 15 bp, synthetic construct
  misc_feature 1..15: note = Description of Artificial Sequence: Synthetic probe
  source 1..15: mol_type = other DNA
cctgctgaaaatgac

SEQ ID NO: 250 - DNA, 15 bp, synthetic construct
  misc_feature 1..15: note = Description of Artificial Sequence: Synthetic probe
  source 1..15: mol_type = other DNA
cctgctgaaaatgac

SEQ ID NO: 251 - DNA, 15 bp, synthetic construct
  misc_feature 1..15: note = Description of Artificial Sequence: Synthetic probe
  source 1..15: mol_type = other DNA
cctgctgaaaatgac

SEQ ID NO: 252 - DNA, 15 bp, synthetic construct
  misc_feature 1..15: note = Description of Artificial Sequence: Synthetic probe
  source 1..15: mol_type = other DNA
cctgctgaaaatgac

SEQ ID NO: 253 - DNA, 15 bp, synthetic construct
  misc_feature 1..15: note = Description of Artificial Sequence: Synthetic probe
  source 1..15: mol_type = other DNA
cctgctgaaaatgac

SEQ ID NO: 254 - DNA, 18 bp, synthetic construct
  misc_feature 1..18: note = Description of Artificial Sequence: Synthetic probe
  source 1..18: mol_type = other DNA
tcgtccacaaaatgattc

SEQ ID NO: 255 - DNA, 18 bp, synthetic construct
  misc_feature 1..18: note = Description of Artificial Sequence: Synthetic probe
  source 1..18: mol_type = other DNA
ccttcgcctgtcctcatg

SEQ ID NO: 256 - DNA, 18 bp, synthetic construct
  misc_feature 1..18: note = Description of Artificial Sequence: Synthetic probe
  source 1..18: mol_type = other DNA
ccttcgcctgtcctcatg

SEQ ID NO: 257 - DNA, 14 bp, synthetic construct
  misc_feature 1..14: note = Description of Artificial Sequence: Synthetic probe
  source 1..14: mol_type = other DNA
cagtgcgcttttcc

SEQ ID NO: 258 - DNA, 14 bp, synthetic construct
  misc_feature 1..14: note = Description of Artificial Sequence: Synthetic probe
  source 1..14: mol_type = other DNA
cagtgcgcttttcc

SEQ ID NO: 259 - DNA, 18 bp, synthetic construct
  misc_feature 1..18: note = Description of Artificial Sequence: Synthetic probe
  source 1..18: mol_type = other DNA
ctgctcaccatcgctatc

SEQ ID NO: 260 - DNA, 17 bp, synthetic construct
  misc_feature 1..17: note = Description of Artificial Sequence: Synthetic probe
  source 1..17: mol_type = other DNA
cctcaggcggctcatag

SEQ ID NO: 261 - DNA, 17 bp, synthetic construct
  misc_feature 1..17: note = Description of Artificial Sequence: Synthetic probe
  source 1..17: mol_type = other DNA
atgggcctccggttcat

SEQ ID NO: 262 - DNA, 16 bp, synthetic construct
  misc_feature 1..16: note = Description of Artificial Sequence: Synthetic probe
  source 1..16: mol_type = other DNA
accggaggcccatcct

SEQ ID NO: 263 - DNA, 16 bp, synthetic construct
  misc_feature 1..16: note = Description of Artificial Sequence: Synthetic probe
  source 1..16: mol_type = other DNA
cacactggaagactcc

SEQ ID NO: 264 - DNA, 16 bp, synthetic construct
  misc_feature 1..16: note = Description of Artificial Sequence: Synthetic probe
  source 1..16: mol_type = other DNA
cacactggaagactcc

SEQ ID NO: 265 - DNA, 15 bp, synthetic construct
  misc_feature 1..15: note = Description of Artificial Sequence: Synthetic probe
  source 1..15: mol_type = other DNA
ctgtgcgccggtctc

SEQ ID NO: 266 - DNA, 16 bp, synthetic construct
  misc_feature 1..16: note = Description of Artificial Sequence: Synthetic probe
  source 1..16: mol_type = other DNA
gctacagagaaatctc

SEQ ID NO: 267 - DNA, 17 bp, synthetic construct
  misc_feature 1..17: note = Description of Artificial Sequence: Synthetic probe
  source 1..17: mol_type = other DNA
gagctgtggtagtggca

SEQ ID NO: 268 - DNA, 16 bp, synthetic construct
  misc_feature 1..16: note = Description of Artificial Sequence: Synthetic probe
  source 1..16: mol_type = other DNA
cactcagagaaggagc

SEQ ID NO: 269 - DNA, 16 bp, synthetic construct
  misc_feature 1..16: note = Description of Artificial Sequence: Synthetic probe
  source 1..16: mol_type = other DNA
catcacgcagctcatg

SEQ ID NO: 270 - DNA, 16 bp, synthetic construct
  misc_feature 1..16: note = Description of Artificial Sequence: Synthetic probe
  source 1..16: mol_type = other DNA
agtttggcccgcccaa

SEQ ID NO: 271 - DNA, 17 bp, synthetic construct
  misc_feature 1..17: note = Description of Artificial Sequence: Synthetic probe
  source 1..17: mol_type = other DNA
ctcgacacagcaggtca

SEQ ID NO: 272 - DNA, 18 bp, synthetic construct
  misc_feature 1..18: note = Description of Artificial Sequence: Synthetic probe
  source 1..18: mol_type = other DNA
gcaggtcaagaggagtac

SEQ ID NO: 273 - DNA, 18 bp, synthetic construct
  misc_feature 1..18: note = Description of Artificial Sequence: Synthetic probe
  source 1..18: mol_type = other DNA
gcaggtcaagaggagtac

SEQ ID NO: 274 - DNA, 14 bp, synthetic construct
  misc_feature 1..14: note = Description of Artificial Sequence: Synthetic probe
  source 1..14: mol_type = other DNA
cgccactagctcca

SEQ ID NO: 275 - DNA, 16 bp, synthetic construct
  misc_feature 1..16: note = Description of Artificial Sequence: Synthetic probe
  source 1..16: mol_type = other DNA
ccacgagctccaacta

SEQ ID NO: 276 - DNA, 14 bp, synthetic construct
  misc_feature 1..14: note = Description of Artificial Sequence: Synthetic probe
  source 1..14: mol_type = other DNA
acgccaccagctcc

SEQ ID NO: 277 - DNA, 13 bp, synthetic construct
  misc_feature 1..13: note = Description of Artificial Sequence: Synthetic probe
  source 1..13: mol_type = other DNA
cgccatcagctcc

SEQ ID NO: 278 - DNA, 13 bp, synthetic construct
  misc_feature 1..13: note = Description of Artificial Sequence: Synthetic probe
  source 1..13: mol_type = other DNA
cgccagcagctcc

SEQ ID NO: 279 - DNA, 14 bp, synthetic construct
  misc_feature 1..14: note = Description of Artificial Sequence: Synthetic probe
  source 1..14: mol_type = other DNA
cgccaccagctcca

SEQ ID NO: 280 - DNA, 15 bp, synthetic construct
  misc_feature 1..15: note = Description of Artificial Sequence: Synthetic probe
  source 1..15: mol_type = other DNA
gctggtgacgtaggc

SEQ ID NO: 281 - DNA, 18 bp, synthetic construct
  misc_feature 1..18: note = Description of Artificial Sequence: Synthetic probe
  source 1..18: mol_type = other DNA
gctggaaaagaagagtac

SEQ ID NO: 282 - DNA, 17 bp, synthetic construct
  misc_feature 1..17: note = Description of Artificial Sequence: Synthetic probe
  source 1..17: mol_type = other DNA
cagctggacaagaagag

SEQ ID NO: 283 - DNA, 17 bp, synthetic construct
  misc_feature 1..17: note = Description of Artificial Sequence: Synthetic probe
  source 1..17: mol_type = other DNA
ttggagcagatggtgtt

SEQ ID NO: 284 - DNA, 16 bp, synthetic construct
  misc_feature 1..16: note = Description of Artificial Sequence: Synthetic probe
  source 1..16: mol_type = other DNA
tggagcaggtgatgtt

SEQ ID NO: 285 - DNA, 13 bp, synthetic construct
  misc_feature 1..13: note = Description of Artificial Sequence: Synthetic probe
  source 1..13: mol_type = other DNA
tgaggcactgccc

SEQ ID NO: 286 - DNA, 26 bp, synthetic construct
  misc_feature 1..26: note = Description of Artificial Sequence: Synthetic probe
  source 1..26: mol_type = other DNA
gaaacacttttcgacatagtgtggtg

SEQ ID NO: 287 - DNA, 20 bp, synthetic construct
  misc_feature 1..20: note = Description of Artificial Sequence: Synthetic probe
  source 1..20: mol_type = other DNA
tgtgtaacagtgcctgcatg

SEQ ID NO: 288 - DNA, 14 bp, synthetic construct
  misc_feature 1..14: note = Description of Artificial Sequence: Synthetic probe
  source 1..14: mol_type = other DNA
gcatgggcagcatg

SEQ ID NO: 289 - DNA, 15 bp, synthetic construct
  misc_feature 1..15: note = Description of Artificial Sequence: Synthetic probe
  source 1..15: mol_type = other DNA
gcatgaaccggaggc

SEQ ID NO: 290 - DNA, 15 bp, synthetic construct
  misc_feature 1..15: note = Description of Artificial Sequence: Synthetic probe
  source 1..15: mol_type = other DNA
catgaaccagaggcc

SEQ ID NO: 291 - DNA, 18 bp, synthetic construct
  misc_feature 1..18: note = Description of Artificial Sequence: Synthetic probe
  source 1..18: mol_type = other DNA
ttgaggtgcgtgtttgtg

SEQ ID NO: 292 - DNA, 16 bp, synthetic construct
  misc_feature 1..16: note = Description of Artificial Sequence: Synthetic probe
  source 1..16: mol_type = other DNA
gctacagtgaaatctc

SEQ ID NO: 293 - DNA, 16 bp, synthetic construct
  misc_feature 1..16: note = Description of Artificial Sequence: Synthetic probe
  source 1..16: mol_type = other DNA
agctgtggcagtggca

SEQ ID NO: 294 - DNA, 16 bp, synthetic construct
  misc_feature 1..16: note = Description of Artificial Sequence: Synthetic probe
  source 1..16: mol_type = other DNA
cactcagaaaaggagc

SEQ ID NO: 295 - DNA, 17 bp, synthetic construct
  misc_feature 1..17: note = Description of Artificial Sequence: Synthetic probe
  source 1..17: mol_type = other DNA
tcatcatgcagctcatg

SEQ ID NO: 296 - DNA, 16 bp, synthetic construct
  misc_feature 1..16: note = Description of Artificial Sequence: Synthetic probe
  source 1..16: mol_type = other DNA
agtttggccagcccaa

SEQ ID NO: 297 - DNA, 17 bp, synthetic construct
  misc_feature 1..17: note = Description of Artificial Sequence: Synthetic probe
  source 1..17: mol_type = other DNA
ctcgacacaggaggtca

SEQ ID NO: 298 - DNA, 17 bp, synthetic construct
  misc_feature 1..17: note = Description of Artificial Sequence: Synthetic probe
  source 1..17: mol_type = other DNA
caggtcacgaggagtac

SEQ ID NO: 299 - DNA, 18 bp, synthetic construct
  misc_feature 1..18: note = Description of Artificial Sequence: Synthetic probe
  source 1..18: mol_type = other DNA
gcaggtcatgaggagtac

SEQ ID NO: 300 - DNA, 13 bp, synthetic construct
  misc_feature 1..13: note = Description of Artificial Sequence: Synthetic probe
  source 1..13: mol_type = other DNA
cgccaccagctcc

SEQ ID NO: 301 - DNA, 16 bp, synthetic construct
  misc_feature 1..16: note = Description of Artificial Sequence: Synthetic probe
  source 1..16: mol_type = other DNA
ccaccagctccaacta

SEQ ID NO: 302 - DNA, 16 bp, synthetic construct
  misc_feature 1..16: note = Description of Artificial Sequence: Synthetic probe
  source 1..16: mol_type = other DNA
acgccacaagctccaa

SEQ ID NO: 303 - DNA, 13 bp, synthetic construct
  misc_feature 1..13: note = Description of Artificial Sequence: Synthetic probe
  source 1..13: mol_type = other DNA
cgccaccagctcc

SEQ ID NO: 304 - DNA, 13 bp, synthetic construct
  misc_feature 1..13: note = Description of Artificial Sequence: Synthetic probe
  source 1..13: mol_type = other DNA
cgccaccagctcc

SEQ ID NO: 305 - DNA, 15 bp, synthetic construct
  misc_feature 1..15: note = Description of Artificial Sequence: Synthetic probe
  source 1..15: mol_type = other DNA
cgccaacagctccaa

SEQ ID NO: 306 - DNA, 15 bp, synthetic construct
  misc_feature 1..15: note = Description of Artificial Sequence: Synthetic probe
  source 1..15: mol_type = other DNA
gctggtggcgtaggc

SEQ ID NO: 307 - DNA, 18 bp, synthetic construct
  misc_feature 1..18: note = Description of Artificial Sequence: Synthetic probe
  source 1..18: mol_type = other DNA
gctggacaagaagagtac

SEQ ID NO: 308 - DNA, 17 bp, synthetic construct
  misc_feature 1..17: note = Description of Artificial Sequence: Synthetic probe
  source 1..17: mol_type = other DNA
cagctggacatgaagag

SEQ ID NO: 309 - DNA, 16 bp, synthetic construct
  misc_feature 1..16: note = Description of Artificial Sequence: Synthetic probe
  source 1..16: mol_type = other DNA
tggagcaggtggtgtt

SEQ ID NO: 310 - DNA, 16 bp, synthetic construct
  misc_feature 1..16: note = Description of Artificial Sequence: Synthetic probe
  source 1..16: mol_type = other DNA
tggagcaggtggtgtt

SEQ ID NO: 311 - DNA, 13 bp, synthetic construct
  misc_feature 1..13: note = Description of Artificial Sequence: Synthetic probe
  source 1..13: mol_type = other DNA
tgaggcgctgccc

SEQ ID NO: 312 - DNA, 27 bp, synthetic construct
  misc_feature 1..27: note = Description of Artificial Sequence: Synthetic probe
  source 1..27: mol_type = other DNA
agaaacactttttgacatagtgtggtg

SEQ ID NO: 313 - DNA, 21 bp, synthetic construct
  misc_feature 1..21: note = Description of Artificial Sequence: Synthetic probe
  source 1..21: mol_type = other DNA
atgtgtaacagttcctgcatg

SEQ ID NO: 314 - DNA, 14 bp, synthetic construct
  misc_feature 1..14: note = Description of Artificial Sequence: Synthetic probe
  source 1..14: mol_type = other DNA
gcatgggcggcatg

SEQ ID NO: 315 - DNA, 16 bp, synthetic construct
  misc_feature 1..16: note = Description of Artificial Sequence: Synthetic probe
  source 1..16: mol_type = other DNA
gcatgaactggaggcc

SEQ ID NO: 316 - DNA, 15 bp, synthetic construct
  misc_feature 1..15: note = Description of Artificial Sequence: Synthetic probe
  source 1..15: mol_type = other DNA
catgaaccggaggcc

SEQ ID NO: 317 - DNA, 20 bp, synthetic construct
  misc_feature 1..20: note = Description of Artificial Sequence: Synthetic probe
  source 1..20: mol_type = other DNA
tttgaggtgtgtgtttgtgc

SEQ ID NO: 318 - DNA, 19 bp, synthetic construct
  misc_feature 1..19: note = Description of Artificial Sequence: Synthetic primer
  source 1..19: mol_type = other DNA
ccctggtagttggagctgg

SEQ ID NO: 319 - DNA, 20 bp, synthetic construct
  misc_feature 1..20: note = Description of Artificial Sequence: Synthetic primer
  source 1..20: mol_type = other DNA
cgctgtggtagttggagctg

SEQ ID NO: 320 - DNA, 19 bp, synthetic construct
  misc_feature 1..19: note = Description of Artificial Sequence: Synthetic primer
  source 1..19: mol_type = other DNA
gcccggtagttggagctgg

SEQ ID NO: 321 - DNA, 20 bp, synthetic construct
  misc_feature 1..20: note = Description of Artificial Sequence: Synthetic primer
  source 1..20: mol_type = other DNA
cgccgtggtagttggagctg

SEQ ID NO: 322 - DNA, 19 bp, synthetic construct
  misc_feature 1..19: note = Description of Artificial Sequence: Synthetic primer
  source 1..19: mol_type = other DNA
ccccagttggagctggtgg

SEQ ID NO: 323 - DNA, 17 bp, synthetic construct
  misc_feature 1..17: note = Description of Artificial Sequence: Synthetic primer
  source 1..17: mol_type = other DNA
gccttgcctacgccaca

SEQ ID NO: 324 - DNA, 18 bp, synthetic construct
  misc_feature 1..18: note = Description of Artificial Sequence: Synthetic primer
  source 1..18: mol_type = other DNA
gcctcttgcctacgccaa

SEQ ID NO: 325 - DNA, 23 bp, synthetic construct
  misc_feature 1..23: note = Description of Artificial Sequence: Synthetic primer
  source 1..23: mol_type = other DNA
aaagaatggtcctgcaccagtaa

SEQ ID NO: 326 - DNA, 22 bp, synthetic construct
  misc_feature 1..22: note = Description of Artificial Sequence: Synthetic primer
  source 1..22: mol_type = other DNA
ggtcctgcaccagtaatatgca

SEQ ID NO: 327 - DNA, 23 bp, synthetic construct
  misc_feature 1..23: note = Description of Artificial Sequence: Synthetic primer
  source 1..23: mol_type = other DNA
aaagaatggtcctgcaccagtaa

SEQ ID NO: 328 - DNA, 23 bp, synthetic construct
  misc_feature 1..23: note = Description of Artificial Sequence: Synthetic primer
  source 1..23: mol_type = other DNA
aaagaatggtcctgcaccagtaa

SEQ ID NO: 329 - DNA, 23 bp, synthetic construct
  misc_feature 1..23: note = Description of Artificial Sequence: Synthetic primer
  source 1..23: mol_type = other DNA
aaagaatggtcctgcaccagtaa

SEQ ID NO: 330 - DNA, 29 bp, synthetic construct
  misc_feature 1..29: note = Description of Artificial Sequence: Synthetic primer
  source 1..29: mol_type = other DNA
gagtttgtattaaaaggtactggtggagt

SEQ ID NO: 331 - DNA, 29 bp, synthetic construct
  misc_feature 1..29: note = Description of Artificial Sequence: Synthetic primer
  source 1..29: mol_type = other DNA
gagtttgtattaaaaggtactggtggagt

SEQ ID NO: 332 - DNA, 19 bp, synthetic construct
  misc_feature 1..19: note = Description of Artificial Sequence: Synthetic primer
  source 1..19: mol_type = other DNA
ccctggtagttggagctgc

SEQ ID NO: 333 - DNA, 20 bp, synthetic construct
  misc_feature 1..20: note = Description of Artificial Sequence: Synthetic primer
  source 1..20: mol_type = other DNA
cgctgtggtagttggagctc

SEQ ID NO: 334 - DNA, 19 bp, synthetic construct
  misc_feature 1..19: note = Description of Artificial Sequence: Synthetic primer
  source 1..19: mol_type = other DNA
gcccggtagttggagctga

SEQ ID NO: 335 - DNA, 20 bp, synthetic construct
  misc_feature 1..20: note = Description of Artificial Sequence: Synthetic primer
  source 1..20: mol_type = other DNA
cgccgtggtagttggagcta

SEQ ID NO: 336 - DNA, 19 bp, synthetic construct
  misc_feature 1..19: note = Description of Artificial Sequence: Synthetic primer
  source 1..19: mol_type = other DNA
ccccagttggagctggtga

SEQ ID NO: 337 - DNA, 17 bp, synthetic construct
  misc_feature 1..17: note = Description of Artificial Sequence: Synthetic primer
  source 1..17: mol_type = other DNA
gccttgcctacgccacc

SEQ ID NO: 338 - DNA, 18 bp, synthetic construct
  misc_feature 1..18: note = Description of Artificial Sequence: Synthetic primer
  source 1..18: mol_type = other DNA
gcctcttgcctacgccac

SEQ ID NO: 339 - DNA, 23 bp, synthetic construct
  misc_feature 1..23: note = Description of Artificial Sequence: Synthetic probe
  source 1..23: mol_type = other DNA
atgcatattaaaacaagatttac

SEQ ID NO: 340 - DNA, 20 bp, synthetic construct
  misc_feature 1..20: note = Description of Artificial Sequence: Synthetic probe
  source 1..20: mol_type = other DNA
aacaatagaggtaaatcttg

SEQ ID NO: 341 - DNA, 23 bp, synthetic construct
  misc_feature 1..23: note = Description of Artificial Sequence: Synthetic probe
  source 1..23: mol_type = other DNA
atgcatattaaaacaagatttac

SEQ ID NO: 342 - DNA, 23 bp, synthetic construct
  misc_feature 1..23: note = Description of Artificial Sequence: Synthetic probe
  source 1..23: mol_type = other DNA
atgcatattaaaacaagatttac

SEQ ID NO: 343 - DNA, 23 bp, synthetic construct
  misc_feature 1..23: note = Description of Artificial Sequence: Synthetic probe
  source 1..23: mol_type = other DNA
atgcatattaaaacaagatttac

SEQ ID NO: 344 - DNA, 20 bp, synthetic construct
  misc_feature 1..20: note = Description of Artificial Sequence: Synthetic probe
  source 1..20: mol_type = other DNA
accttatgtgtgacatgttc

SEQ ID NO: 345 - DNA, 20 bp, synthetic construct
  misc_feature 1..20: note = Description of Artificial Sequence: Synthetic probe
  source 1..20: mol_type = other DNA
accttatgtgtgacatgttc

SEQ ID NO: 346 - DNA, 16 bp, synthetic construct
  misc_feature 1..16: note = Description of Artificial Sequence: Synthetic probe
  source 1..16: mol_type = other DNA
tggagctgctggcgta

SEQ ID NO: 347 - DNA, 16 bp, synthetic construct
  misc_feature 1..16: note = Description of Artificial Sequence: Synthetic probe
  source 1..16: mol_type = other DNA
tggagctcgtggcgta

SEQ ID NO: 348 - DNA, 17 bp, synthetic construct
  misc_feature 1..17: note = Description of Artificial Sequence: Synthetic probe
  source 1..17: mol_type = other DNA
ttggagctgatggcgta

SEQ ID NO: 349 - DNA, 20 bp, synthetic construct
  misc_feature 1..20: note = Description of Artificial Sequence: Synthetic probe
  source 1..20: mol_type = other DNA
tagttggagctagtggcgta

SEQ ID NO: 350 - DNA, 17 bp, synthetic construct
  misc_feature 1..17: note = Description of Artificial Sequence: Synthetic probe
  source 1..17: mol_type = other DNA
ttggagctggtgacgta

SEQ ID NO: 351 - DNA, 14 bp, synthetic construct
  misc_feature 1..14: note = Description of Artificial Sequence: Synthetic probe
  source 1..14: mol_type = other DNA
cgccaccagctcca

SEQ ID NO: 352 - DNA, 15 bp, synthetic construct
  misc_feature 1..15: note = Description of Artificial Sequence: Synthetic probe
  source 1..15: mol_type = other DNA
ctacgccaccagctc

SEQ ID NO: 353 - DNA, 16 bp, synthetic construct
  misc_feature 1..16: note = Description of Artificial Sequence: Synthetic probe
  source 1..16: mol_type = other DNA
tggagctggtggcgta

SEQ ID NO: 354 - DNA, 16 bp, synthetic construct
  misc_feature 1..16: note = Description of Artificial Sequence: Synthetic probe
  source 1..16: mol_type = other DNA
tggagctggtggcgta

SEQ ID NO: 355 - DNA, 16 bp, synthetic construct
  misc_feature 1..16: note = Description of Artificial Sequence: Synthetic probe
  source 1..16: mol_type = other DNA
tggagctggtggcgta

SEQ ID NO: 356 - DNA, 16 bp, synthetic construct
  misc_feature 1..16: note = Description of Artificial Sequence: Synthetic probe
  source 1..16: mol_type = other DNA
tggagctggtggcgta

SEQ ID NO: 357 - DNA, 16 bp, synthetic construct
  misc_feature 1..16: note = Description of Artificial Sequence: Synthetic probe
  source 1..16: mol_type = other DNA
tggagctggtggcgta

SEQ ID NO: 358 - DNA, 14 bp, synthetic construct
  misc_feature 1..14: note = Description of Artificial Sequence: Synthetic probe
  source 1..14: mol_type = other DNA
cgccacaagctcca

SEQ ID NO: 359 - DNA, 14 bp, synthetic construct
  misc_feature 1..14: note = Description of Artificial Sequence: Synthetic probe
  source 1..14: mol_type = other DNA
tacgccaacagctc

SEQ ID NO: 360 - DNA, 21 bp, synthetic construct
  misc_feature 1..21: note = Description of Artificial Sequence: Synthetic primer
  source 1..21: mol_type = other DNA
tgattttggtctagctacaga

SEQ ID NO: 361 - DNA, 16 bp, synthetic construct
  misc_feature 1..16: note = Description of Artificial Sequence: Synthetic primer
  source 1..16: mol_type = other DNA
gagaaggagctgtggt

SEQ ID NO: 362 - DNA, 18 bp, synthetic construct
  misc_feature 1..18: note = Description of Artificial Sequence: Synthetic primer
  source 1..18: mol_type = other DNA
tgcctttaccactcagag

SEQ ID NO: 363 - DNA, 15 bp, synthetic construct
  misc_feature 1..15: note = Description of Artificial Sequence: Synthetic primer
  source 1..15: mol_type = other DNA
cgtgcagctcatcac

SEQ ID NO: 364 - DNA, 14 bp, synthetic construct
  misc_feature 1..14: note = Description of Artificial Sequence: Synthetic primer
  source 1..14: mol_type = other DNA
cagcagtttggccc

SEQ ID NO: 365 - DNA, 18 bp, synthetic construct
  misc_feature 1..18: note = Description of Artificial Sequence: Synthetic primer
  source 1..18: mol_type = other DNA
ggatattctcgacacagc

SEQ ID NO: 366 - DNA, 15 bp, synthetic construct
  misc_feature 1..15: note = Description of Artificial Sequence: Synthetic primer
  source 1..15: mol_type = other DNA
gacacagcaggtcaa

SEQ ID NO: 367 - DNA, 15 bp, synthetic construct
  misc_feature 1..15: note = Description of Artificial Sequence: Synthetic primer
  source 1..15: mol_type = other DNA
cttgcctacgccact

SEQ ID NO: 368 - DNA, 16 bp, synthetic construct
  misc_feature 1..16: note = Description of Artificial Sequence: Synthetic primer
  source 1..16: mol_type = other DNA
ctcttgcctacgccat

SEQ ID NO: 369 - DNA, 17 bp, synthetic construct
  misc_feature 1..17: note = Description of Artificial Sequence: Synthetic primer
  source 1..17: mol_type = other DNA
gtagttggagctggtga

SEQ ID NO: 370 - DNA, 19 bp, synthetic construct
  misc_feature 1..19: note = Description of Artificial Sequence: Synthetic primer
  source 1..19: mol_type = other DNA
atactggatacagctggaa

SEQ ID NO: 371 - DNA, 17 bp, synthetic construct
  misc_feature 1..17: note = Description of Artificial Sequence: Synthetic primer
  source 1..17: mol_type = other DNA
tggatacagctggacaa

SEQ ID NO: 372 - DNA, 20 bp, synthetic construct
  misc_feature 1..20: note = Description of Artificial Sequence: Synthetic primer
  source 1..20: mol_type = other DNA
gattttggtctagctacagt

SEQ ID NO: 373 - DNA, 15 bp, synthetic construct
  misc_feature 1..15: note = Description of Artificial Sequence: Synthetic primer
  source 1..15: mol_type = other DNA
agaaggagctgtggc

SEQ ID NO: 374 - DNA, 18 bp, synthetic construct
  misc_feature 1..18: note = Description of Artificial Sequence: Synthetic primer
  source 1..18: mol_type = other DNA
tgcctttaccactcagaa

SEQ ID NO: 375 - DNA, 15 bp, synthetic construct
  misc_feature 1..15: note = Description of Artificial Sequence: Synthetic primer
  source 1..15: mol_type = other DNA
cgtgcagctcatcat

SEQ ID NO: 376 - DNA, 14 bp, synthetic construct
  misc_feature 1..14: note = Description of Artificial Sequence: Synthetic primer
  source 1..14: mol_type = other DNA
cagcagtttggcca

SEQ ID NO: 377 - DNA, 19 bp, synthetic construct
  misc_feature 1..19: note = Description of Artificial Sequence: Synthetic primer
  source 1..19: mol_type = other DNA
tggatattctcgacacagg

SEQ ID NO: 378 - DNA, 15 bp, synthetic construct
  misc_feature 1..15: note = Description of Artificial Sequence: Synthetic primer
  source 1..15: mol_type = other DNA
gacacagcaggtcac

SEQ ID NO: 379 - DNA, 14 bp, synthetic construct
  misc_feature 1..14: note = Description of Artificial Sequence: Synthetic primer
  source 1..14: mol_type = other DNA
ttgcctacgccacc

SEQ ID NO: 380 - DNA, 15 bp, synthetic construct
  misc_feature 1..15: note = Description of Artificial Sequence: Synthetic primer
  source 1..15: mol_type = other DNA
tcttgcctacgccac

SEQ ID NO: 381 - DNA, 16 bp, synthetic construct
  misc_feature 1..16: note = Description of Artificial Sequence: Synthetic primer
  source 1..16: mol_type = other DNA
tagttggagctggtgg

SEQ ID NO: 382 - DNA, 19 bp, synthetic construct
  misc_feature 1..19: note = Description of Artificial Sequence: Synthetic primer
  source 1..19: mol_type = other DNA
atactggatacagctggac

SEQ ID NO: 383 - DNA, 18 bp, synthetic construct
  misc_feature 1..18: note = Description of Artificial Sequence: Synthetic primer
  source 1..18: mol_type = other DNA
ctggatacagctggacat

SEQ ID NO: 384 - DNA, 24 bp, synthetic construct
  misc_feature 1..24: note = Description of Artificial Sequence: Synthetic primer
  source 1..24: mol_type = other DNA
gcctgattttggtctagctacaga

SEQ ID NO: 385 - DNA, 19 bp, synthetic construct
  misc_feature 1..19: note = Description of Artificial Sequence: Synthetic primer
  source 1..19: mol_type = other DNA
cgcgagaaggagctgtggt

SEQ ID NO: 386 - DNA, 21 bp, synthetic construct
  misc_feature 1..21: note = Description of Artificial Sequence: Synthetic primer
  source 1..21: mol_type = other DNA
ccgtgcctttaccactcagag

SEQ ID NO: 387 - DNA, 18 bp, synthetic construct
  misc_feature 1..18: note = Description of Artificial Sequence: Synthetic primer
  source 1..18: mol_type = other DNA
gcgcgtgcagctcatcac

SEQ ID NO: 388 - DNA, 17 bp, synthetic construct
  misc_feature 1..17: note = Description of Artificial Sequence: Synthetic primer
  source 1..17: mol_type = other DNA
cggcagcagtttggccc

SEQ ID NO: 389 - DNA, 21 bp, synthetic construct
  misc_feature 1..21: note = Description of Artificial Sequence: Synthetic primer
  source 1..21: mol_type = other DNA
gccggatattctcgacacagc

SEQ ID NO: 390 - DNA, 18 bp, synthetic construct
  misc_feature 1..18: note = Description of Artificial Sequence: Synthetic primer
  source 1..18: mol_type = other DNA
gcggacacagcaggtcaa

SEQ ID NO: 391 - DNA, 18 bp, synthetic construct
  misc_feature 1..18: note = Description of Artificial Sequence: Synthetic primer
  source 1..18: mol_type = other DNA
cgccttgcctacgccact

SEQ ID NO: 392 - DNA, 19 bp, synthetic construct
  misc_feature 1..19: note = Description of Artificial Sequence: Synthetic primer
  source 1..19: mol_type = other DNA
cgcctcttgcctacgccat

SEQ ID NO: 393 - DNA, 20 bp, synthetic construct
  misc_feature 1..20: note = Description of Artificial Sequence: Synthetic primer
  source 1..20: mol_type = other DNA
cgcgtagttggagctggtga

SEQ ID NO: 394 - DNA, 22 bp, synthetic construct
  misc_feature 1..22: note = Description of Artificial Sequence: Synthetic primer
  source 1..22: mol_type = other DNA
ccgatactggatacagctggaa

SEQ ID NO: 395 - DNA, 20 bp, synthetic construct
  misc_feature 1..20: note = Description of Artificial Sequence: Synthetic primer
  source 1..20: mol_type = other DNA
ccgtggatacagctggacaa

SEQ ID NO: 396 - DNA, 23 bp, synthetic construct
  misc_feature 1..23: note = Description of Artificial Sequence: Synthetic primer
  source 1..23: mol_type = other DNA
ccggattttggtctagctacagt

SEQ ID NO: 397 - DNA, 18 bp, synthetic construct
  misc_feature 1..18: note = Description of Artificial Sequence: Synthetic primer
  source 1..18: mol_type = other DNA
gccagaaggagctgtggc

SEQ ID NO: 398 - DNA, 21 bp, synthetic construct
  misc_feature 1..21: note = Description of Artificial Sequence: Synthetic primer
  source 1..21: mol_type = other DNA
ccgtgcctttaccactcagaa

SEQ ID NO: 399 - DNA, 18 bp, synthetic construct
  misc_feature 1..18: note = Description of Artificial Sequence: Synthetic primer
  source 1..18: mol_type = other DNA
gcgcgtgcagctcatcat

SEQ ID NO: 400 - DNA, 17 bp, synthetic construct
  misc_feature 1..17: note = Description of Artificial Sequence: Synthetic primer
  source 1..17: mol_type = other DNA
cggcagcagtttggcca

SEQ ID NO: 401 - DNA, 22 bp, synthetic construct
  misc_feature 1..22: note = Description of Artificial Sequence: Synthetic primer
  source 1..22: mol_type = other DNA
cgctggatattctcgacacagg

SEQ ID NO: 402 - DNA, 18 bp, synthetic construct
  misc_feature 1..18: note = Description of Artificial Sequence: Synthetic primer
  source 1..18: mol_type = other DNA
gcggacacagcaggtcac

SEQ ID NO: 403 - DNA, 17 bp, synthetic construct
  misc_feature 1..17: note = Description of Artificial Sequence: Synthetic primer
  source 1..17: mol_type = other DNA
gcgttgcctacgccacc

SEQ ID NO: 404 - DNA, 18 bp, synthetic construct
  misc_feature 1..18: note = Description of Artificial Sequence: Synthetic primer
  source 1..18: mol_type = other DNA
gcgtcttgcctacgccac

SEQ ID NO: 405 - DNA, 19 bp, synthetic construct
  misc_feature 1..19: note = Description of Artificial Sequence: Synthetic primer
  source 1..19: mol_type = other DNA
gcctagttggagctggtgg

SEQ ID NO: 406 - DNA, 22 bp, synthetic construct
  misc_feature 1..22: note = Description of Artificial Sequence: Synthetic primer
  source 1..22: mol_type = other DNA
ccgatactggatacagctggac

SEQ ID NO: 407 - DNA, 21 bp, synthetic construct
  misc_feature 1..21: note = Description of Artificial Sequence: Synthetic primer
  source 1..21: mol_type = other DNA
cgcctggatacagctggacat

SEQ ID NO: 408 - DNA, 26 bp, synthetic construct
  misc_feature 1..26: note = Description of Artificial Sequence: Synthetic primer
  source 1..26: mol_type = other DNA
agcctcaattcttaccatccacaaaa

SEQ ID NO: 409 - DNA, 22 bp, synthetic construct
  misc_feature 1..22: note = Description of Artificial Sequence: Synthetic primer
  source 1..22: mol_type = other DNA
catggaaccagacagaaaagcg

SEQ ID NO: 410 - DNA, 22 bp, synthetic construct
  misc_feature 1..22: note = Description of Artificial Sequence: Synthetic primer
  source 1..22: mol_type = other DNA
gtcactggcagcaacagtctta

SEQ ID NO: 411 - DNA, 25 bp, synthetic construct
  misc_feature 1..25: note = Description of Artificial Sequence: Synthetic primer
  source 1..25: mol_type = other DNA
tgggagccaatattgtctttgtgtt

SEQ ID NO: 412 - DNA, 23 bp, synthetic construct
  misc_feature 1..23: note = Description of Artificial Sequence: Synthetic primer
  source 1..23: mol_type = other DNA
aggaacgtactggtgaaaacacc

SEQ ID NO: 413 - DNA, 20 bp, synthetic construct
  misc_feature 1..20: note = Description of Artificial Sequence: Synthetic primer
  source 1..20: mol_type = other DNA
ccctccccagtcctcatgta

SEQ ID NO: 414 - DNA, 41 bp, synthetic construct; identical to SEQ ID NO: 220
  misc_feature 1..41: note = Description of Artificial Sequence: Synthetic primer
  source 1..41: mol_type = other DNA

SEQ ID NO: 415 - DNA, 28 bp, synthetic construct
  misc_feature 1..28: note = Description of Artificial Sequence: Synthetic primer
  source 1..28: mol_type = other DNA
tgtgtgacatgttctaatatagtcacat

SEQ ID NO: 416 - DNA, 28 bp, synthetic construct
  misc_feature 1..28: note = Description of Artificial Sequence: Synthetic primer
  source 1..28: mol_type = other DNA
tgtgtgacatgttctaatatagtcacat

SEQ ID NO: 417 - DNA, 22 bp, synthetic construct
  misc_feature 1..22: note = Description of Artificial Sequence: Synthetic primer
  source 1..22: mol_type = other DNA
ggtcctgcaccagtaatatgca

SEQ ID NO: 418 - DNA, 29 bp, synthetic construct
  misc_feature 1..29: note = Description of Artificial Sequence: Synthetic primer
  source 1..29: mol_type = other DNA
gcaaatgacttgctattattgatggcaaa

SEQ ID NO: 419 - DNA, 29 bp, synthetic construct
  misc_feature 1..29: note = Description of Artificial Sequence: Synthetic primer
  source 1..29: mol_type = other DNA
gcaaatgacttgctattattgatggcaaa

SEQ ID NO: 420 - DNA, 19 bp, synthetic construct
  misc_feature 1..19: note = Description of Artificial Sequence: Synthetic probe
  source 1..19: mol_type = other DNA
cagacaactgttcaaactg

SEQ ID NO: 421 - DNA, 16 bp, synthetic construct
  misc_feature 1..16: note = Description of Artificial Sequence: Synthetic probe
  source 1..16: mol_type = other DNA
ctggcagcaacagtct

SEQ ID NO: 422 - DNA, 17 bp, synthetic construct
  misc_feature 1..17: note = Description of Artificial Sequence: Synthetic probe
  source 1..17: mol_type = other DNA
tagtggcaccagaatgg

SEQ ID NO: 423 - DNA, 16 bp, synthetic construct
  misc_feature 1..16: note = Description of Artificial Sequence: Synthetic probe
  source 1..16: mol_type = other DNA
cccttcggctgcctcc

SEQ ID NO: 424 - DNA, 17 bp, synthetic construct
  misc_feature 1..17: note = Description of Artificial Sequence: Synthetic probe
  source 1..17: mol_type = other DNA
cagcatgtcaagatcac

SEQ ID NO: 425 - DNA, 17 bp, synthetic construct
  misc_feature 1..17: note = Description of Artificial Sequence: Synthetic probe
  source 1..17: mol_type = other DNA
ctggtccctcattgcac

SEQ ID NO: 426 - DNA, 16 bp, synthetic construct
  misc_feature 1..16: note = Description of Artificial Sequence: Synthetic probe
  source 1..16: mol_type = other DNA
ccctccccagtcctca

SEQ ID NO: 427 - DNA, 15 bp, synthetic construct
  misc_feature 1..15: note = Description of Artificial Sequence: Synthetic probe
  source 1..15: mol_type = other DNA
cctgctgaaaatgac

SEQ ID NO: 428 - DNA, 15 bp, synthetic construct
  misc_feature 1..15: note = Description of Artificial Sequence: Synthetic probe
  source 1..15: mol_type = other DNA
cctgctgaaaatgac

SEQ ID NO: 429 - DNA, 18 bp, synthetic construct
  misc_feature 1..18: note = Description of Artificial Sequence: Synthetic probe
  source 1..18: mol_type = other DNA
tcgtccacaaaatgattc

SEQ ID NO: 430 - DNA, 18 bp, synthetic construct
  misc_feature 1..18: note = Description of Artificial Sequence: Synthetic probe
  source 1..18: mol_type = other DNA
ccttcgcctgtcctcatg

SEQ ID NO: 431 - DNA, 18 bp, synthetic construct
  misc_feature 1..18: note = Description of Artificial Sequence: Synthetic probe
  source 1..18: mol_type = other DNA
ccttcgcctgtcctcatg

SEQ ID NO: 432 - DNA, 14 bp, synthetic construct
  misc_feature 1..14: note = Description of Artificial Sequence: Synthetic probe
  source 1..14: mol_type = other DNA
ctacagagaaatct

SEQ ID NO: 433 - DNA, 14 bp, synthetic construct
  misc_feature 1..14: note = Description of Artificial Sequence: Synthetic probe
  source 1..14: mol_type = other DNA
ctgtggtagtggca

SEQ ID NO: 434 - DNA, 14 bp, synthetic construct
  misc_feature 1..14: note = Description of Artificial Sequence: Synthetic probe
  source 1..14: mol_type = other DNA
ctcagagaaggagc

SEQ ID NO: 435 - DNA, 13 bp, synthetic construct
  misc_feature 1..13: note = Description of Artificial Sequence: Synthetic probe
  source 1..13: mol_type = other DNA
atcacgcagctca

SEQ ID NO: 436 - DNA, 11 bp, synthetic construct
  misc_feature 1..11: note = Description of Artificial Sequence: Synthetic probe
  source 1..11: mol_type = other DNA
ggcccgcccaa

SEQ ID NO: 437 - DNA, 14 bp, synthetic construct
  misc_feature 1..14: note = Description of Artificial Sequence: Synthetic probe
  source 1..14: mol_type = other DNA
gacacagcaggtca

SEQ ID NO: 438 - DNA, 16 bp, synthetic construct
  misc_feature 1..16: note = Description of Artificial Sequence: Synthetic probe
  source 1..16: mol_type = other DNA
caggtcaagaggagta

SEQ ID NO: 439 - DNA, 13 bp, synthetic construct
  misc_feature 1..13: note = Description of Artificial Sequence: Synthetic probe
  source 1..13: mol_type = other DNA
gccactagctcca

SEQ ID NO: 440 - DNA, 12 bp, synthetic construct
  misc_feature 1..12: note = Description of Artificial Sequence: Synthetic probe
  source 1..12: mol_type = other DNA
gccatcagctcc

SEQ ID NO: 441 - DNA, 13 bp, synthetic construct
  misc_feature 1..13: note = Description of Artificial Sequence: Synthetic probe
  source 1..13: mol_type = other DNA
tggtgacgtaggc

SEQ ID NO: 442 - DNA, 15 bp, synthetic construct
  misc_feature 1..15: note = Description of Artificial Sequence: Synthetic probe
  source 1..15: mol_type = other DNA
gctggaaaagaagag

SEQ ID NO: 443 - DNA, 14 bp, synthetic construct
  misc_feature 1..14: note = Description of Artificial Sequence: Synthetic probe
  source 1..14: mol_type = other DNA
ctggacaagaagag

SEQ ID NO: 444 - DNA, 14 bp, synthetic construct
  misc_feature 1..14: note = Description of Artificial Sequence: Synthetic probe
  source 1..14: mol_type = other DNA
ctacagtgaaatct

SEQ ID NO: 445 - DNA, 13 bp, synthetic construct
  misc_feature 1..13: note = Description of Artificial Sequence: Synthetic probe
  source 1..13: mol_type = other DNA
tgtggcagtggca

SEQ ID NO: 446 - DNA, 14 bp, synthetic construct
  misc_feature 1..14: note = Description of Artificial Sequence: Synthetic probe
  source 1..14: mol_type = other DNA
ctcagaaaaggagc

SEQ ID NO: 447 - DNA, 14 bp, synthetic construct
  misc_feature 1..14: note = Description of Artificial Sequence: Synthetic probe
  source 1..14: mol_type = other DNA
catcatgcagctca

SEQ ID NO: 448 - DNA, 12 bp, synthetic construct
  misc_feature 1..12: note = Description of Artificial Sequence: Synthetic probe
  source 1..12: mol_type = other DNA
tggccagcccaa

SEQ ID NO: 449 - DNA, 14 bp, synthetic construct
  misc_feature 1..14: note = Description of Artificial Sequence: Synthetic probe
  source 1..14: mol_type = other DNA
gacacaggaggtca

SEQ ID NO: 450 - DNA, 15 bp, synthetic construct
  misc_feature 1..15: note = Description of Artificial Sequence: Synthetic probe
  source 1..15: mol_type = other DNA
aggtcacgaggagta

SEQ ID NO: 451 - DNA, 12 bp, synthetic construct
  misc_feature 1..12: note = Description of Artificial Sequence: Synthetic probe
  source 1..12: mol_type = other DNA
ccaccagctcca

SEQ ID NO: 452 - DNA, 11 bp, synthetic construct
  misc_feature 1..11: note = Description of Artificial Sequence: Synthetic probe
  source 1..11: mol_type = other DNA
ccaccagctcc

SEQ ID NO: 453 - DNA, 13 bp, synthetic construct
  misc_feature 1..13: note = Description of Artificial Sequence: Synthetic probe
  source 1..13: mol_type = other DNA
tggtggcgtaggc

SEQ ID NO: 454 - DNA, 14 bp, synthetic construct
  misc_feature 1..14: note = Description of Artificial Sequence: Synthetic probe
  source 1..14: mol_type = other DNA
ctggacaagaagag

SEQ ID NO: 455 - DNA, 14 bp, synthetic construct
  misc_feature 1..14: note = Description of Artificial Sequence: Synthetic probe
  source 1..14: mol_type = other DNA
ctggacatgaagag

SEQ ID NO: 456 - DNA, 22 bp, synthetic construct
  misc_feature 1..22: note = Description of Artificial Sequence: Synthetic primer
  source 1..22: mol_type = other DNA
gtgattttggtctagctacaga

SEQ ID NO: 457 - DNA, 19 bp, synthetic construct
  misc_feature 1..19: note = Description of Artificial Sequence: Synthetic primer
  source 1..19: mol_type = other DNA
tcagagaaggagctgtggt

SEQ ID NO: 458 - DNA, 21 bp, synthetic construct
  misc_feature 1..21: note = Description of Artificial Sequence: Synthetic primer
  source 1..21: mol_type = other DNA
gattgcctttaccactcagag

SEQ ID NO: 459 - DNA, 18 bp, synthetic construct
  misc_feature 1..18: note = Description of Artificial Sequence: Synthetic primer
  source 1..18: mol_type = other DNA
caccgtgcagctcatcac

SEQ ID NO: 460 - DNA, 18 bp, synthetic construct
  misc_feature 1..18: note = Description of Artificial Sequence: Synthetic primer
  source 1..18: mol_type = other DNA
cacccagcagtttggccc

SEQ ID NO: 461 - DNA, 20 bp, synthetic construct
  misc_feature 1..20: note = Description of Artificial Sequence: Synthetic primer
  source 1..20: mol_type = other DNA
ttggatattctcgacacagc

SEQ ID NO: 462 - DNA, 18 bp, synthetic construct
  misc_feature 1..18: note = Description of Artificial Sequence: Synthetic primer
  source 1..18: mol_type = other DNA
ctcgacacagcaggtcaa

SEQ ID NO: 463 - DNA, 18 bp, synthetic construct
  misc_feature 1..18: note = Description of Artificial Sequence: Synthetic primer
  source 1..18: mol_type = other DNA
actcttgcctacgccact

SEQ ID NO: 464 - DNA, 18 bp, synthetic construct
  misc_feature 1..18: note = Description of Artificial Sequence: Synthetic primer
  source 1..18: mol_type = other DNA
cactcttgcctacgccat

SEQ ID NO: 465 - DNA, 19 bp, synthetic construct
  misc_feature 1..19: note = Description of Artificial Sequence: Synthetic primer
  source 1..19: mol_type = other DNA
tggtagttggagctggtga

SEQ ID NO: 466 - DNA, 20 bp, synthetic construct
  misc_feature 1..20: note = Description of Artificial Sequence: Synthetic primer
  source 1..20: mol_type = other DNA
catactggatacagctggaa

SEQ ID NO: 467 - DNA, 21 bp, synthetic construct
  misc_feature 1..21: note = Description of Artificial Sequence: Synthetic primer
  source 1..21: mol_type = other DNA
atactggatacagctggacaa

SEQ ID NO: 468 - DNA, 22 bp, synthetic construct
  misc_feature 1..22: note = Description of Artificial Sequence: Synthetic primer
  source 1..22: mol_type = other DNA
gtgattttggtctagctacagt

SEQ ID NO: 469 - DNA, 19 bp, synthetic construct
  misc_feature 1..19: note = Description of Artificial Sequence: Synthetic primer
  source 1..19: mol_type = other DNA
tcagagaaggagctgtggc

SEQ ID NO: 470 - DNA, 21 bp, synthetic construct
  misc_feature 1..21: note = Description of Artificial Sequence: Synthetic primer
  source 1..21: mol_type = other DNA
gattgcctttaccactcagaa

SEQ ID NO: 471 - DNA, 18 bp, synthetic construct
  misc_feature 1..18: note = Description of Artificial Sequence: Synthetic primer
  source 1..18: mol_type = other DNA
caccgtgcagctcatcat

SEQ ID NO: 472 - DNA, 18 bp, synthetic construct
  misc_feature 1..18: note = Description of Artificial Sequence: Synthetic primer
  source 1..18: mol_type = other DNA
cacccagcagtttggcca

SEQ ID NO: 473 - DNA, 21 bp, synthetic construct
  misc_feature 1..21: note = Description of Artificial Sequence: Synthetic primer
  source 1..21: mol_type = other DNA
cttggatattctcgacacagg

SEQ ID NO: 474 - DNA, 19 bp, synthetic construct
  misc_feature 1..19: note = Description of Artificial Sequence: Synthetic primer
  source 1..19: mol_type = other DNA
tctcgacacagcaggtcac

SEQ ID NO: 475 - DNA, 18 bp, synthetic construct
  misc_feature 1..18: note = Description of Artificial Sequence: Synthetic primer
  source 1..18: mol_type = other DNA
actcttgcctacgccacc

SEQ ID NO: 476 - DNA, 18 bp, synthetic construct
  misc_feature 1..18: note = Description of Artificial Sequence: Synthetic primer
  source 1..18: mol_type = other DNA
cactcttgcctacgccac

SEQ ID NO: 477 - DNA, 19 bp, synthetic construct
  misc_feature 1..19: note = Description of Artificial Sequence: Synthetic primer
  source 1..19: mol_type = other DNA
tggtagttggagctggtgg

SEQ ID NO: 478 - DNA, 20 bp, synthetic construct
  misc_feature 1..20: note = Description of Artificial Sequence: Synthetic primer
  source 1..20: mol_type = other DNA
catactggatacagctggac

SEQ ID NO: 479 - DNA, 20 bp, synthetic construct
  misc_feature 1..20: note = Description of Artificial Sequence: Synthetic primer
  source 1..20: mol_type = other DNA
tactggatacagctggacat

SEQ ID NO: 480 - DNA, 26 bp, synthetic construct
  misc_feature 1..26: note = Description of Artificial Sequence: Synthetic primer
  source 1..26: mol_type = other DNA
agcctcaattcttaccatccacaaaa

SEQ ID NO: 481 - DNA, 22 bp, synthetic construct
  misc_feature 1..22: note = Description of Artificial Sequence: Synthetic primer
  source 1..22: mol_type = other DNA
catggaaccagacagaaaagcg

SEQ ID NO: 482 - DNA, 22 bp, synthetic construct
  misc_feature 1..22: note = Description of Artificial Sequence: Synthetic primer
  source 1..22: mol_type = other DNA
gtcactggcagcaacagtctta

SEQ ID NO: 483 - DNA, 25 bp, synthetic construct
  misc_feature 1..25: note = Description of Artificial Sequence: Synthetic primer
  source 1..25: mol_type = other DNA
tgggagccaatattgtctttgtgtt

SEQ ID NO: 484 - DNA, 23 bp, synthetic construct
  misc_feature 1..23: note = Description of Artificial Sequence: Synthetic primer
  source 1..23: mol_type = other DNA
aggaacgtactggtgaaaacacc

SEQ ID NO: 485 - DNA, 20 bp, synthetic construct
  misc_feature 1..20: note = Description of Artificial Sequence: Synthetic primer
  source 1..20: mol_type = other DNA
ccctccccagtcctcatgta

SEQ ID NO: 486 - DNA, 41 bp, synthetic construct; identical to SEQ ID NO: 220
  misc_feature 1..41: note = Description of Artificial Sequence: Synthetic primer
  source 1..41: mol_type = other DNA

SEQ ID NO: 487 - DNA, 28 bp, synthetic construct
  misc_feature 1..28: note = Description of Artificial Sequence: Synthetic primer
  source 1..28: mol_type = other DNA
tgtgtgacatgttctaatatagtcacat

SEQ ID NO: 488 - DNA, 28 bp, synthetic construct
  misc_feature 1..28: note = Description of Artificial Sequence: Synthetic primer
  source 1..28: mol_type = other DNA
tgtgtgacatgttctaatatagtcacat

SEQ ID NO: 489 - DNA, 22 bp, synthetic construct
  misc_feature 1..22: note = Description of Artificial Sequence: Synthetic primer
  source 1..22: mol_type = other DNA
ggtcctgcaccagtaatatgca

SEQ ID NO: 490 - DNA, 29 bp, synthetic construct
  misc_feature 1..29: note = Description of Artificial Sequence: Synthetic primer
  source 1..29: mol_type = other DNA
gcaaatgacttgctattattgatggcaaa

SEQ ID NO: 491 - DNA, 29 bp, synthetic construct
  misc_feature 1..29: note = Description of Artificial Sequence: Synthetic primer
  source 1..29: mol_type = other DNA
gcaaatgacttgctattattgatggcaaa

SEQ ID NO: 492 - DNA, 19 bp, synthetic construct
  misc_feature 1..19: note = Description of Artificial Sequence: Synthetic probe
  source 1..19: mol_type = other DNA
cagacaactgttcaaactg

SEQ ID NO: 493 - DNA, 16 bp, synthetic construct
  misc_feature 1..16: note = Description of Artificial Sequence: Synthetic probe
  source 1..16: mol_type = other DNA
ctggcagcaacagtct

SEQ ID NO: 494 - DNA, 17 bp, synthetic construct
  misc_feature 1..17: note = Description of Artificial Sequence: Synthetic probe
  source 1..17: mol_type = other DNA
tagtggcaccagaatgg

SEQ ID NO: 495 - DNA, 16 bp, synthetic construct
  misc_feature 1..16: note = Description of Artificial Sequence: Synthetic probe
  source 1..16: mol_type = other DNA
cccttcggctgcctcc

SEQ ID NO: 496 - DNA, 17 bp, synthetic construct
  misc_feature 1..17: note = Description of Artificial Sequence: Synthetic probe
  source 1..17: mol_type = other DNA
cagcatgtcaagatcac

SEQ ID NO: 497 - DNA, 17 bp, synthetic construct
  misc_feature 1..17: note = Description of Artificial Sequence: Synthetic probe
  source 1..17: mol_type = other DNA
ctggtccctcattgcac

SEQ ID NO: 498 - DNA, 16 bp, synthetic construct
  misc_feature 1..16: note = Description of Artificial Sequence: Synthetic probe
  source 1..16: mol_type = other DNA
ccctccccagtcctca

SEQ ID NO: 499 - DNA, 15 bp, synthetic construct
  misc_feature 1..15: note = Description of Artificial Sequence: Synthetic probe
  source 1..15: mol_type = other DNA
cctgctgaaaatgac

SEQ ID NO: 500 - DNA, 15 bp, synthetic construct
  misc_feature 1..15: note = Description of Artificial Sequence: Synthetic probe
  source 1..15: mol_type = other DNA
cctgctgaaaatgac

SEQ ID NO: 501 - DNA, 18 bp, synthetic construct
  misc_feature 1..18: note = Description of Artificial Sequence: Synthetic probe
  source 1..18: mol_type = other DNA
tcgtccacaaaatgattc

SEQ ID NO: 502 - DNA, 18 bp, synthetic construct
  misc_feature 1..18: note = Description of Artificial Sequence: Synthetic probe
  source 1..18: mol_type = other DNA
ccttcgcctgtcctcatg

SEQ ID NO: 503 - DNA, 18 bp, synthetic construct
  misc_feature 1..18: note = Description of Artificial Sequence: Synthetic probe
  source 1..18: mol_type = other DNA
ccttcgcctgtcctcatg

SEQ ID NO: 504 - DNA, 21 bp, synthetic construct
  misc_feature 1..21: note = Description of Artificial Sequence: Synthetic probe
  source 1..21: mol_type = other DNA
gctacagagaaatctcgatgg

SEQ ID NO: 505 - DNA, 17 bp, synthetic construct
  misc_feature 1..17: note = Description of Artificial Sequence: Synthetic probe
  source 1..17: mol_type = other DNA
gagctgtggtagtggca

SEQ ID NO: 506 - DNA, 20 bp, synthetic construct
  misc_feature 1..20: note = Description of Artificial Sequence: Synthetic probe
  source 1..20: mol_type = other DNA
ttaccactcagagaaggagc

SEQ ID NO: 507 - DNA, 17 bp, synthetic construct
  misc_feature 1..17: note = Description of Artificial Sequence: Synthetic probe
  source 1..17: mol_type = other DNA
gctcatcacgcagctca

SEQ ID NO: 508 - DNA, 16 bp, synthetic construct
  misc_feature 1..16: note = Description of Artificial Sequence: Synthetic probe
  source 1..16: mol_type = other DNA
ttggcccgcccaaaat

SEQ ID NO: 509 - DNA, 18 bp, synthetic construct
  misc_feature 1..18: note = Description of Artificial Sequence: Synthetic probe
  source 1..18: mol_type = other DNA
gacacagcaggtcacgag

SEQ ID NO: 510 - DNA, 20 bp, synthetic construct
  misc_feature 1..20: note = Description of Artificial Sequence: Synthetic probe
  source 1..20: mol_type = other DNA
cagcaggtcaagaggagtac

SEQ ID NO: 511 - DNA, 19 bp, synthetic construct
  misc_feature 1..19: note = Description of Artificial Sequence: Synthetic probe
  source 1..19: mol_type = other DNA
ctacgccactagctccaac

SEQ ID NO: 512 - DNA, 16 bp, synthetic construct
  misc_feature 1..16: note = Description of Artificial Sequence: Synthetic probe
  source 1..16: mol_type = other DNA
ctacgccatcagctcc

SEQ ID NO: 513 - DNA, 17 bp, synthetic construct
  misc_feature 1..17: note = Description of Artificial Sequence: Synthetic probe
  source 1..17: mol_type = other DNA
ctggtgacgtaggcaag

SEQ ID NO: 514 - DNA, 22 bp, synthetic construct
  misc_feature 1..22: note = Description of Artificial Sequence: Synthetic probe
  source 1..22: mol_type = other DNA
ggatacagctggaaaagaagag

SEQ ID NO: 515 - DNA, 20 bp, synthetic construct
  misc_feature 1..20: note = Description of Artificial Sequence: Synthetic probe
  source 1..20: mol_type = other DNA
agctggacaagaagagtaca

SEQ ID NO: 516 - DNA, 22 bp, synthetic construct
  misc_feature 1..22: note = Description of Artificial Sequence: Synthetic probe
  source 1..22: mol_type = other DNA
tagctacagtgaaatctcgatg

SEQ ID NO: 517 - DNA, 15 bp, synthetic construct
  misc_feature 1..15: note = Description of Artificial Sequence: Synthetic probe
  source 1..15: mol_type = other DNA
gagctgtggcagtgg

SEQ ID NO: 518 - DNA, 21 bp, synthetic construct
  misc_feature 1..21: note = Description of Artificial Sequence: Synthetic probe
  source 1..21: mol_type = other DNA
ttaccactcagaaaaggagct

SEQ ID NO: 519 - DNA, 20 bp, synthetic construct
  misc_feature 1..20: note = Description of Artificial Sequence: Synthetic probe
  source 1..20: mol_type = other DNA
cagctcatcatgcagctcat

SEQ ID NO: 520 - DNA, 19 bp, synthetic construct
  misc_feature 1..19: note = Description of Artificial Sequence: Synthetic probe
  source 1..19: mol_type = other DNA
gtttggccagcccaaaatc

SEQ ID NO: 521 - DNA, 18 bp, synthetic construct
  misc_feature 1..18: note = Description of Artificial Sequence: Synthetic probe
  source 1..18: mol_type = other DNA
gacacaggaggtcaggag

SEQ ID NO: 522 - DNA, 19 bp, synthetic construct
  misc_feature 1..19: note = Description of Artificial Sequence: Synthetic probe
  source 1..19: mol_type = other DNA
cagcaggtcacgaggagta

SEQ ID NO: 523 - DNA, 17 bp, synthetic construct
  misc_feature 1..17: note = Description of Artificial Sequence: Synthetic probe
  source 1..17: mol_type = other DNA
tacgccaccagctccaa

SEQ ID NO: 524 - DNA, 16 bp, synthetic construct
  misc_feature 1..16: note = Description of Artificial Sequence: Synthetic probe
  source 1..16: mol_type = other DNA
tacgccaccagctcca

SEQ ID NO: 525 - DNA, 17 bp, synthetic construct
  misc_feature 1..17: note = Description of Artificial Sequence: Synthetic probe
  source 1..17: mol_type = other DNA
ctggtggcgtaggcaag

SEQ ID NO: 526 - DNA, 21 bp, synthetic construct
  misc_feature 1..21: note = Description of Artificial Sequence: Synthetic probe
  source 1..21: mol_type = other DNA
atacagctggacaagaagagt

SEQ ID NO: 527 - DNA, 20 bp, synthetic construct
  misc_feature 1..20: note = Description of Artificial Sequence: Synthetic probe
  source 1..20: mol_type = other DNA
agctggacatgaagagtaca

SEQ ID NO: 528 - DNA, 22 bp, synthetic construct
  misc_feature 1..22: note = Description of Artificial Sequence: Synthetic primer
  source 1..22: mol_type = other DNA
ccccttttggtctagctacaga

SEQ ID NO: 529 - DNA, 18 bp, synthetic construct
  misc_feature 1..18: note = Description of Artificial Sequence: Synthetic primer
  source 1..18: mol_type = other DNA
gcccgaaggagctgtggt

SEQ ID NO: 530 - DNA, 20 bp, synthetic construct
  misc_feature 1..20: note = Description of Artificial Sequence: Synthetic primer
  source 1..20: mol_type = other DNA
cgcccctttaccactcagag

SEQ ID NO: 531 - DNA, 18 bp, synthetic construct
  misc_feature 1..18: note = Description of Artificial Sequence: Synthetic primer
  source 1..18: mol_type = other DNA
gcgggtgcagctcatcac

SEQ ID NO: 532 - DNA, 17 bp, synthetic construct
  misc_feature 1..17: note = Description of Artificial Sequence: Synthetic primer
  source 1..17: mol_type = other DNA
cgggagcagtttggccc

SEQ ID NO: 533 - DNA, 21 bp, synthetic construct
  misc_feature 1..21: note = Description of Artificial Sequence: Synthetic primer
  source 1..21: mol_type = other DNA
gcccgatattctcgacacagc

SEQ ID NO: 534 - DNA, 18 bp, synthetic construct
  misc_feature 1..18: note = Description of Artificial Sequence: Synthetic primer
  source 1..18: mol_type = other DNA
gcggacacagcaggtcaa

SEQ ID NO: 535 - DNA, 16 bp, synthetic construct
  misc_feature 1..16: note = Description of Artificial Sequence: Synthetic primer
  source 1..16: mol_type = other DNA
cggtgcctacgccact

SEQ ID NO: 536 - DNA, 17 bp, synthetic construct
  misc_feature 1..17: note = Description of Artificial Sequence: Synthetic primer
  source 1..17: mol_type = other DNA
cgccttgcctacgccat

SEQ ID NO: 537 - DNA, 19 bp, synthetic construct
  misc_feature 1..19: note = Description of Artificial Sequence: Synthetic primer
  source 1..19: mol_type = other DNA
gcccagttggagctggtga

SEQ ID NO: 538 - DNA, 21 bp, synthetic construct
  misc_feature 1..21: note = Description of Artificial Sequence: Synthetic primer
  source 1..21: mol_type = other DNA
cgccactggatacagctggaa

SEQ ID NO: 539 - DNA, 20 bp, synthetic construct
  misc_feature 1..20: note = Description of Artificial Sequence: Synthetic primer
  source 1..20: mol_type = other DNA
ccgcggatacagctggacaa

SEQ ID NO: 540 - DNA, 22 bp, synthetic construct
  misc_feature 1..22: note = Description of Artificial Sequence: Synthetic primer
  source 1..22: mol_type = other DNA
ccccttttggtctagctacagt

SEQ ID NO: 541 - DNA, 18 bp, synthetic construct
  misc_feature 1..18: note = Description of Artificial Sequence: Synthetic primer
  source 1..18: mol_type = other DNA
gcccgaaggagctgtggc

SEQ ID NO: 542 - DNA, 20 bp, synthetic construct
  misc_feature 1..20: note = Description of Artificial Sequence: Synthetic primer
  source 1..20: mol_type = other DNA
cgcccctttaccactcagaa

SEQ ID NO: 543 - DNA, 18 bp, synthetic construct
  misc_feature 1..18: note = Description of Artificial Sequence: Synthetic primer
  source 1..18: mol_type = other DNA
gcgggtgcagctcatcat

SEQ ID NO: 544 - DNA, 17 bp, synthetic construct
  misc_feature 1..17: note = Description of Artificial Sequence: Synthetic primer
  source 1..17: mol_type = other DNA
cgggagcagtttggcca

SEQ ID NO: 545 - DNA, 21 bp, synthetic construct
  misc_feature 1..21: note = Description of Artificial Sequence: Synthetic primer
  source 1..21: mol_type = other DNA
gcccgatattctcgacacagg

SEQ ID NO: 546 - DNA, 18 bp, synthetic construct
  misc_feature 1..18: note = Description of Artificial Sequence: Synthetic primer
  source 1..18: mol_type = other DNA
gcggacacagcaggtcac

SEQ ID NO: 547 - DNA, 16 bp, synthetic construct
  misc_feature 1..16: note = Description of Artificial Sequence: Synthetic primer
  source 1..16: mol_type = other DNA
cggtgcctacgccacc

SEQ ID NO: 548 - DNA, 17 bp, synthetic construct
  misc_feature 1..17: note = Description of Artificial Sequence: Synthetic primer
  source 1..17: mol_type = other DNA
cgccttgcctacgccac

SEQ ID NO: 549 - DNA, 19 bp, synthetic construct
  misc_feature 1..19: note = Description of Artificial Sequence: Synthetic primer
  source 1..19: mol_type = other DNA
gcccagttggagctggtgg

SEQ ID NO: 550 - DNA, 21 bp, synthetic construct
  misc_feature 1..21: note = Description of Artificial Sequence: Synthetic primer
  source 1..21: mol_type = other DNA
cgccactggatacagctggac

SEQ ID NO: 551 - DNA, 20 bp, synthetic construct
  misc_feature 1..20: note = Description of Artificial Sequence: Synthetic primer
  source 1..20: mol_type = other DNA
ccgcggatacagctggacat

SEQ ID NO: 552 - DNA, 26 bp, synthetic construct
  misc_feature 1..26: note = Description of Artificial Sequence: Synthetic primer
  source 1..26: mol_type = other DNA
agcctcaattcttaccatccacaaaa

SEQ ID NO: 553 - DNA, 22 bp, synthetic construct
  misc_feature 1..22: note = Description of Artificial Sequence: Synthetic primer
  source 1..22: mol_type = other DNA
catggaaccagacagaaaagcg

SEQ ID NO: 554 - DNA, 22 bp, synthetic construct
  misc_feature 1..22: note = Description of Artificial Sequence: Synthetic primer
  source 1..22: mol_type = other DNA
gtcactggcagcaacagtctta

SEQ ID NO: 555 - DNA, 25 bp, synthetic construct
  misc_feature 1..25: note = Description of Artificial Sequence: Synthetic primer
  source 1..25: mol_type = other DNA
tgggagccaatattgtctttgtgtt

SEQ ID NO: 556 - DNA, 23 bp, synthetic construct
  misc_feature 1..23: note = Description of Artificial Sequence: Synthetic primer
  source 1..23: mol_type = other DNA
aggaacgtactggtgaaaacacc

SEQ ID NO: 557 - DNA, 20 bp, synthetic construct
  misc_feature 1..20: note = Description of Artificial Sequence: Synthetic primer
  source 1..20: mol_type = other DNA
ccctccccagtcctcatgta

SEQ ID NO: 558 - DNA, 41 bp, synthetic construct; identical to SEQ ID NO: 220
  misc_feature 1..41: note = Description of Artificial Sequence: Synthetic primer
  source 1..41: mol_type = other DNA

SEQ ID NO: 559 - DNA, 28 bp, synthetic construct
  misc_feature 1..28: note = Description of Artificial Sequence: Synthetic primer
  source 1..28: mol_type = other DNA
tgtgtgacatgttctaatatagtcacat

SEQ ID NO: 560 - DNA, 28 bp, synthetic construct
  misc_feature 1..28: note = Description of Artificial Sequence: Synthetic primer
  source 1..28: mol_type = other DNA
tgtgtgacatgttctaatatagtcacat

SEQ ID NO: 561 - DNA, 22 bp, synthetic construct
  misc_feature 1..22: note = Description of Artificial Sequence: Synthetic primer
  source 1..22: mol_type = other DNA
ggtcctgcaccagtaatatgca

SEQ ID NO: 562 - DNA, 29 bp, synthetic construct
  misc_feature 1..29: note = Description of Artificial Sequence: Synthetic primer
  source 1..29: mol_type = other DNA
gcaaatgacttgctattattgatggcaaa

SEQ ID NO: 563 - DNA, 29 bp, synthetic construct
  misc_feature 1..29: note = Description of Artificial Sequence: Synthetic primer
  source 1..29: mol_type = other DNA
gcaaatgacttgctattattgatggcaaa

SEQ ID NO: 564 - DNA, 19 bp, synthetic construct
  misc_feature 1..19: note = Description of Artificial Sequence: Synthetic probe
  source 1..19: mol_type = other DNA
cagacaactgttcaaactg

SEQ ID NO: 565 - DNA, 16 bp, synthetic construct
  misc_feature 1..16: note = Description of Artificial Sequence: Synthetic probe
  source 1..16: mol_type = other DNA
ctggcagcaacagtct

SEQ ID NO: 566 - DNA, 17 bp, synthetic construct
  misc_feature 1..17: note = Description of Artificial Sequence: Synthetic probe
  source 1..17: mol_type = other DNA
tagtggcaccagaatgg

SEQ ID NO: 567 - DNA, 16 bp, synthetic construct
  misc_feature 1..16: note = Description of Artificial Sequence: Synthetic probe
  source 1..16: mol_type = other DNA
cccttcggctgcctcc

SEQ ID NO: 568 - DNA, 17 bp, synthetic construct
  misc_feature 1..17: note = Description of Artificial Sequence: Synthetic probe
  source 1..17: mol_type = other DNA
cagcatgtcaagatcac

SEQ ID NO: 569 - DNA, 17 bp, synthetic construct
  misc_feature 1..17: note = Description of Artificial Sequence: Synthetic probe
  source 1..17: mol_type = other DNA
ctggtccctcattgcac

SEQ ID NO: 570 - DNA, 16 bp, synthetic construct
  misc_feature 1..16: note = Description of Artificial Sequence: Synthetic probe
  source 1..16: mol_type = other DNA
ccctccccagtcctca

SEQ ID NO: 571 - DNA, 15 bp, synthetic construct
  misc_feature 1..15: note = Description of Artificial Sequence: Synthetic probe
  source 1..15: mol_type = other DNA
cctgctgaaaatgac

SEQ ID NO: 572 - DNA, 15 bp, synthetic construct
  misc_feature 1..15: note = Description of Artificial Sequence: Synthetic probe
  source 1..15: mol_type = other DNA
cctgctgaaaatgac

SEQ ID NO: 573 - DNA, 18 bp, synthetic construct
  misc_feature 1..18: note = Description of Artificial Sequence: Synthetic probe
  source 1..18: mol_type = other DNA
tcgtccacaaaatgattc

SEQ ID NO: 574 - DNA, 18 bp, synthetic construct
  misc_feature 1..18: note = Description of Artificial Sequence: Synthetic probe
  source 1..18: mol_type = other DNA
ccttcgcctgtcctcatg

SEQ ID NO: 575 - DNA, 18 bp, synthetic construct
  misc_feature 1..18: note = Description of Artificial Sequence: Synthetic probe
  source 1..18: mol_type = other DNA
ccttcgcctgtcctcatg

SEQ ID NO: 576 - DNA, 14 bp, synthetic construct
  misc_feature 1..14: note = Description of Artificial Sequence: Synthetic probe
  source 1..14: mol_type = other DNA
ctacagagaaatct

SEQ ID NO: 577 - DNA, 14 bp, synthetic construct
  misc_feature 1..14: note = Description of Artificial Sequence: Synthetic probe
  source 1..14: mol_type = other DNA
ctgtggtagtggca

SEQ ID NO: 578 - DNA, 14 bp, synthetic construct
  misc_feature 1..14: note = Description of Artificial Sequence: Synthetic probe
  source 1..14: mol_type = other DNA
ctcagagaaggagc

SEQ ID NO: 579 - DNA, 13 bp, synthetic construct
  misc_feature 1..13: note = Description of Artificial Sequence: Synthetic probe
  source 1..13: mol_type = other DNA
atcacgcagctca

SEQ ID NO: 580 - DNA, 11 bp, synthetic construct
  misc_feature 1..11: note = Description of Artificial Sequence: Synthetic probe
  source 1..11: mol_type = other DNA
ggcccgcccaa

SEQ ID NO: 581 - DNA, 14 bp, synthetic construct
  misc_feature 1..14: note = Description of Artificial Sequence: Synthetic probe
  source 1..14: mol_type = other DNA
gacacagcaggtca

SEQ ID NO: 582 - DNA, 16 bp, synthetic construct
  misc_feature 1..16: note = Description of Artificial Sequence: Synthetic probe
  source 1..16: mol_type = other DNA
caggtcaagaggagta

SEQ ID NO: 583 - DNA, 13 bp, synthetic construct
  misc_feature 1..13: note = Description of Artificial Sequence: Synthetic probe
  source 1..13: mol_type = other DNA
gccactagctcca

SEQ ID NO: 584 - DNA, 12 bp, synthetic construct
  misc_feature 1..12: note = Description of Artificial Sequence: Synthetic probe
  source 1..12: mol_type = other DNA
gccatcagctcc

SEQ ID NO: 585 - DNA, 13 bp, synthetic construct
  misc_feature 1..13: note = Description of Artificial Sequence: Synthetic probe
  source 1..13: mol_type = other DNA
tggtgacgtaggc

SEQ ID NO: 586 - DNA, 15 bp, synthetic construct
  misc_feature 1..15: note = Description of Artificial Sequence: Synthetic probe
  source 1..15: mol_type = other DNA
gctggaaaagaagag

SEQ ID NO: 587 - DNA, 14 bp, synthetic construct
  misc_feature 1..14: note = Description of Artificial Sequence: Synthetic probe
  source 1..14: mol_type = other DNA
ctggacaagaagag

SEQ ID NO: 588 - DNA, 14 bp, synthetic construct
  misc_feature 1..14: note = Description of Artificial Sequence: Synthetic probe
  source 1..14: mol_type = other DNA
ctacagtgaaatct

SEQ ID NO: 589 - DNA, 13 bp, synthetic construct
  misc_feature 1..13: note = Description of Artificial Sequence: Synthetic probe
  source 1..13: mol_type = other DNA
tgtggcagtggca

SEQ ID NO: 590 - DNA, 14 bp, synthetic construct
  misc_feature 1..14: note = Description of Artificial Sequence: Synthetic probe
  source 1..14: mol_type = other DNA
ctcagaaaaggagc

SEQ ID NO: 591 - DNA, 14 bp, synthetic construct
  misc_feature 1..14: note = Description of Artificial Sequence: Synthetic probe
  source 1..14: mol_type = other DNA
catcatgcagctca

SEQ ID NO: 592 - DNA, 12 bp, synthetic construct
  misc_feature 1..12: note = Description of Artificial Sequence: Synthetic probe
  source 1..12: mol_type = other DNA
tggccagcccaa

SEQ ID NO: 593 - DNA, 14 bp, synthetic construct
  misc_feature 1..14: note = Description of Artificial Sequence: Synthetic probe
  source 1..14: mol_type = other DNA
gacacaggaggtca

SEQ ID NO: 594 - DNA, 15 bp, synthetic construct
  misc_feature 1..15: note = Description of Artificial Sequence: Synthetic probe
  source 1..15: mol_type = other DNA
aggtcacgaggagta

SEQ ID NO: 595 - DNA, 12 bp, synthetic construct
  misc_feature 1..12: note = Description of Artificial Sequence: Synthetic probe
  source 1..12: mol_type = other DNA
ccaccagctcca

SEQ ID NO: 596 - DNA, 11 bp, synthetic construct
  misc_feature 1..11: note = Description of Artificial Sequence: Synthetic probe
  source 1..11: mol_type = other DNA
ccaccagctcc

SEQ ID NO: 597 - DNA, 13 bp, synthetic construct
  misc_feature 1..13: note = Description of Artificial Sequence: Synthetic probe
  source 1..13: mol_type = other DNA
tggtggcgtaggc

SEQ ID NO: 598 - DNA, 14 bp, synthetic construct
  misc_feature 1..14: note = Description of Artificial Sequence: Synthetic probe
  source 1..14: mol_type = other DNA
ctggacaagaagag

SEQ ID NO: 599 - DNA, 14 bp, synthetic construct
  misc_feature 1..14: note = Description of Artificial Sequence: Synthetic probe
  source 1..14: mol_type = other DNA
ctggacatgaagag

SEQ ID NO: 600 - DNA, 22 bp, synthetic construct
  misc_feature 1..22: note = Description of Artificial Sequence: Synthetic primer
  source 1..22: mol_type = other DNA
ccccttttggtctagctacaga

SEQ ID NO: 601 - DNA, 18 bp, synthetic construct
  misc_feature 1..18: note = Synthetic primer
  modified_base 18: mod_base = OTHER; note = uracil
  source 1..18: mol_type = other DNA
gcccgaaggagctgtggt

SEQ ID NO: 602 - DNA, 20 bp, synthetic construct
  misc_feature 1..20: note = Description of Artificial Sequence: Synthetic primer
  source 1..20: mol_type = other DNA
cgcccctttaccactcagag

SEQ ID NO: 603 - DNA, 18 bp, synthetic construct
  misc_feature 1..18: note = Description of Artificial Sequence: Synthetic primer
  source 1..18: mol_type = other DNA
gcgggtgcagctcatcac

SEQ ID NO: 604 - DNA, 17 bp, synthetic construct
  misc_feature 1..17: note = Description of Artificial Sequence: Synthetic primer
  source 1..17: mol_type = other DNA
cgggagcagtttggccc

SEQ ID NO: 605 - DNA, 21 bp, synthetic construct
  misc_feature 1..21: note = Description of Artificial Sequence: Synthetic primer
  source 1..21: mol_type = other DNA
gcccgatattctcgacacagc

SEQ ID NO: 606 - DNA, 26 bp, synthetic construct
  misc_feature 1..26: note = Description of Artificial Sequence: Synthetic primer
  source 1..26: mol_type = other DNA
agcctcaattcttaccatccacaaaa

SEQ ID NO: 607 - DNA, 22 bp, synthetic construct
  misc_feature 1..22: note = Description of Artificial Sequence: Synthetic primer
  source 1..22: mol_type = other DNA
catggaaccagacagaaaagcg

SEQ ID NO: 608 - DNA, 22 bp, synthetic construct
  misc_feature 1..22: note = Description of Artificial Sequence: Synthetic primer
  source 1..22: mol_type = other DNA
gtcactggcagcaacagtctta

SEQ ID NO: 609 - DNA, 25 bp, synthetic construct
  misc_feature 1..25: note = Description of Artificial Sequence: Synthetic primer
  source 1..25: mol_type = other DNA
tgggagccaatattgtctttgtgtt

SEQ ID NO: 610 - DNA, 23 bp, synthetic construct
  misc_feature 1..23: note = Description of Artificial Sequence: Synthetic primer
  source 1..23: mol_type = other DNA
aggaacgtactggtgaaaacacc

SEQ ID NO: 611 - DNA, 20 bp, synthetic construct
  misc_feature 1..20: note = Description of Artificial Sequence: Synthetic primer
  source 1..20: mol_type = other DNA
ccctccccagtcctcatgta

SEQ ID NO: 612 - DNA, 14 bp, synthetic construct
  misc_feature 1..14: note = Description of Artificial Sequence: Synthetic probe
  source 1..14: mol_type = other DNA
ctacagagaaatct

SEQ ID NO: 613 - DNA, 14 bp, synthetic construct
  misc_feature 1..14: note = Description of Artificial Sequence: Synthetic probe
  source 1..14: mol_type = other DNA
ctgtggtagtggca

SEQ ID NO: 614 - DNA, 14 bp, synthetic construct
  misc_feature 1..14: note = Description of Artificial Sequence: Synthetic probe
  source 1..14: mol_type = other DNA
ctcagagaaggagc

SEQ ID NO: 615 - DNA, 13 bp, synthetic construct
  misc_feature 1..13: note = Description of Artificial Sequence: Synthetic probe
  source 1..13: mol_type = other DNA
atcacgcagctca

SEQ ID NO: 616 - DNA, 11 bp, synthetic construct
  misc_feature 1..11: note = Description of Artificial Sequence: Synthetic probe
  source 1..11: mol_type = other DNA
ggcccgcccaa

SEQ ID NO: 617 - DNA, 14 bp, synthetic construct
  misc_feature 1..14: note = Description of Artificial Sequence: Synthetic probe
  source 1..14: mol_type = other DNA
gacacagcaggtca

SEQ ID NO: 618 - DNA, 22 bp, synthetic construct
  misc_feature 1..22: note = Synthetic primer
  modified_base 22: mod_base = OTHER; note = uracil
  source 1..22: mol_type = other DNA
ccccttttggtctagctacagt

SEQ ID NO: 619 - DNA, 18 bp, synthetic construct
  misc_feature 1..18: note = Description of Artificial Sequence: Synthetic primer
  source 1..18: mol_type = other DNA
gcccgaaggagctgtggc

SEQ ID NO: 620 - DNA, 20 bp, synthetic construct
  misc_feature 1..20: note = Description of Artificial Sequence: Synthetic primer
  source 1..20: mol_type = other DNA
cgcccctttaccactcagaa

SEQ ID NO: 621 - DNA, 18 bp, synthetic construct
  misc_feature 1..18: note = Synthetic primer
  modified_base 18: mod_base = OTHER; note = uracil
  source 1..18: mol_type = other DNA
gcgggtgcagctcatcat

SEQ ID NO: 622 - DNA, 17 bp, synthetic construct
  misc_feature 1..17: note = Description of Artificial Sequence: Synthetic primer
  source 1..17: mol_type = other DNA
cgggagcagtttggcca

SEQ ID NO: 623 - DNA, 21 bp, synthetic construct
  misc_feature 1..21: note = Description of Artificial Sequence: Synthetic primer
  source 1..21: mol_type = other DNA
gcccgatattctcgacacagg

SEQ ID NO: 624 - DNA, 19 bp, synthetic construct
  misc_feature 1..19: note = Description of Artificial Sequence: Synthetic probe
  source 1..19: mol_type = other DNA
cagacaactgttcaaactg

SEQ ID NO: 625 - DNA, 16 bp, synthetic construct
  misc_feature 1..16: note = Description of Artificial Sequence: Synthetic probe
  source 1..16: mol_type = other DNA
ctggcagcaacagtct

SEQ ID NO: 626 - DNA, 17 bp, synthetic construct
  misc_feature 1..17: note = Description of Artificial Sequence: Synthetic probe
  source 1..17: mol_type = other DNA
tagtggcaccagaatgg

SEQ ID NO: 627 - DNA, 16 bp, synthetic construct
  misc_feature 1..16: note = Description of Artificial Sequence: Synthetic probe
  source 1..16: mol_type = other DNA
cccttcggctgcctcc

SEQ ID NO: 628 - DNA, 17 bp, synthetic construct
  misc_feature 1..17: note = Description of Artificial Sequence: Synthetic probe
  source 1..17: mol_type = other DNA
cagcatgtcaagatcac

SEQ ID NO: 629 - DNA, 17 bp, synthetic construct
  misc_feature 1..17: note = Description of Artificial Sequence: Synthetic probe
  source 1..17: mol_type = other DNA
ctggtccctcattgcac

SEQ ID NO: 630 - DNA, 14 bp, synthetic construct
  misc_feature 1..14: note = Description of Artificial Sequence: Synthetic probe
  source 1..14: mol_type = other DNA
ctacagtgaaatct

SEQ ID NO: 631 - DNA, 13 bp, synthetic construct
  misc_feature 1..13: note = Description of Artificial Sequence: Synthetic probe
  source 1..13: mol_type = other DNA
tgtggcagtggca

SEQ ID NO: 632 - DNA, 14 bp, synthetic construct
  misc_feature 1..14: note = Description of Artificial Sequence: Synthetic probe
  source 1..14: mol_type = other DNA
ctcagaaaaggagc

SEQ ID NO: 633 - DNA, 14 bp, synthetic construct
  misc_feature 1..14: note = Description of Artificial Sequence: Synthetic probe
  source 1..14: mol_type = other DNA
catcatgcagctca

SEQ ID NO: 634 - DNA, 12 bp, synthetic construct
  misc_feature 1..12: note = Description of Artificial Sequence: Synthetic probe
  source 1..12: mol_type = other DNA
tggccagcccaa

SEQ ID NO: 635 - DNA, 14 bp, synthetic construct
  misc_feature 1..14: note = Description of Artificial Sequence: Synthetic probe
  source 1..14: mol_type = other DNA
gacacaggaggtca

(19) United States
(12) Patent Application Publication
(10) Pub. No.: US 2024/0132946 A1
(43) Pub. Date: Apr. 25, 2024

(54) METHODS, COMPOSITIONS, AND KITS FOR DETECTING ALLELIC VARIANTS

(71) Applicant: LIFE TECHNOLOGIES CORPORATION, Carlsbad, CA (US)

(72) Inventors: Caifu CHEN, Palo Alto, CA (US); Ruoying TAN, Palo Alto, CA (US)

(21) Appl. No.: 18/087,259

(22) Filed: Dec. 22, 2022

Related U.S. Application Data
(63) Continuation of application No. 15/930,101, filed on May 12, 2020, now Pat. No. 11,572,585.
Application No. 15/930,101 is a continuation of application No. 16/109,191, filed on Aug. 22, 2018, now Pat. No. 10,689,694.
Application No. 16/109,191 is a continuation of application No. 14/856,448, filed on Sep. 16, 2015, now Pat. No. 10,081,833.
Application No. 14/856,448 is a continuation of application No. 12/748,329, filed on Mar. 26, 2010, now abandoned.
Application No. 12/748,329 is a continuation-in-part of application No. 12/641,321, filed on Dec. 17, 2009, now Pat. No. 9,534,255.
(60) Provisional application No. 61/138,521, filed on Dec. 17, 2008.
(60) Provisional application No. 61/258,582, filed on Nov. 5, 2009.
(60) Provisional application No. 61/253,501, filed on Oct. 20, 2009.
(60) Provisional application No. 61/251,623, filed on Oct. 14, 2009.
(60) Provisional application No. 61/186,775, filed on Jun. 12, 2009.
(60) Provisional application No. 61/164,230, filed on Mar. 27, 2009.

(51) Int. Cl. C12Q 1/6858; C12Q 1/686; C12Q 1/6883

(52) U.S. Cl. CPC C12Q 1/6858; C12Q 1/686; C12Q 1/6883; C12Q 2600/156

(57) ABSTRACT

In some embodiments, the present inventions relates generally to compositions, methods and kits for use in discriminating sequence variation between different alleles. More specifically, in some embodiments, the present invention provides for compositions, methods and kits for quantitating rare (e.g., mutant) allelic variants, such as SNPs, or nucleotide (NT) insertions or deletions, in samples comprising abundant (e.g., wild type) allelic variants with high specificity and selectivity. In particular, in some embodiments, the invention relates to a highly selective method for mutation detection referred to as competitive allele-specific TaqMan PCR (“cast-PCR”).

### CROSS REFERENCE TO RELATED APPLICATIONS

The present application is a continuation of U.S. application Ser. No. 15/930,101, filed May 12, 2020, which is a continuation of U.S. application Ser. No. 16/109,191, filed Aug. 22, 2018 (now U.S. Pat. No. 10,689,694), which is a continuation of U.S. application Ser. No. 14/856,448 (now U.S. Pat. No. 10,081,833), filed Sep. 16, 2015, which is a continuation of U.S. application Ser. No. 12/748,329 (now abandoned), filed Mar. 26, 2010, which is continuation-in-part of U.S. application Ser. No. 12/641,321 (now U.S. Pat. No. 9,534,255), filed Dec. 17, 2009, which claims the benefit of and priority to U.S. Provisional Application Nos: 61/138,521, filed Dec. 17, 2008; 61/258,582, filed Nov. 5, 2009; 61/253,501, filed Oct. 20, 2009; 61/251,623, filed Oct. 14, 2009; 61/186,775, filed Jun. 12, 2009; and 61/164,230, filed Mar. 27, 2009, all of which are incorporated herein by reference in their entireties.

### SEQUENCE LISTING

The instant application contains a Sequence Listing which has been submitted via EFS-Web and is hereby incorporated by reference in its entirety. Said ASCII copy, created on Jun. 24, 2010, is named LT000051CIP.txt and is 156,489 bytes in size.

### BACKGROUND

Single nucleotide polymorphisms (SNPs) are the most common type of genetic diversity in the human genome, occurring at a frequency of about one SNP in 1,000 nucleotides or less in human genomic DNA (Kwok, P-Y, Ann Rev Genom Hum Genet 2001, 2: 235-258). SNPs have been implicated in genetic disorders, susceptibility to different diseases, predisposition to adverse reactions to drugs, and for use in forensic investigations. Thus, SNP (or rare mutation) detection provides great potentials in diagnosing early phase diseases, such as detecting circulating tumor cells in blood, for prenatal diagnostics, as well as for detection of disease-associated mutations in a mixed cell population.

Numerous approaches for SNP genotyping have been developed based on methods involving hybridization, ligation, or DNA polymerases (Chen, X., and Sullivan, PF, The Pharmacogeonomics Journal 2003, 3, 77-96.). For example, allele-specific polymerase chain reaction (AS-PCR) is a widely used strategy for detecting DNA sequence variation (Wu DY, Ugozzoli L, Pal BK, Wallace RB., Proc Natl Acad Sci U S A 1989;86:2757-2760). AS-PCR, as its name implies, is a PCR-based method whereby one or both primers are designed to anneal at sites of sequence variations which allows for the ability to differentiate among different alleles of the same gene. AS-PCR exploits the fidelity of DNA polymerases, which extend primers with a mismatched 3′ base at much lower efficiency, from 100 to 100,000 fold less efficient, than that with a matched 3′ base (Chen, X., and Sullivan, PF, The Pharmacogeonomics Journal 2003; 3:77-96). The difficulty in extending mismatched primers results in diminished PCR amplification that can be readily detected.

The specificity and selectivity of AS-PCR, however, is largely dependent on the nature of exponential amplification of PCR which makes the decay of allele discriminating power rapid. Even though primers are designed to match a specific variant to selectively amplify only that variant, in actuality significant mismatched amplification often occurs. Moreover, the ability of AS-PCR to differentiate between allelic variants can be influenced by the type of mutation or the sequence surrounding the mutation or SNP (Ayyadevara S, Thaden JJ, Shmookler Reis RJ., Anal Biochem 2000;284:11-18), the amount of allelic variants present in the sample, as well as the ratio between alternative alleles. Collectively, these factors are often responsible for the frequent appearance of false-positive results, leading many researchers to attempt to increase the reliability of AS-PCR (Orou A, Fechner B, Utermann G, Menzel HJ., Hum Mutat 1995;6:163-169)(Imyanitov EN, Busboy KG, Suspitsin EN, Kuligina ES, Belogubova EV, Grigoriev MY, et al., Biotechniques 2002;33:484-490)(McKinzie PB, Parsons BL. Detection of rare K-ras codon 12 mutations using allele-specific competitive blocker PCR. Mutat Res 2002; 517:209-220) (Latorra D, Campbell K, Wolter A, Hurley JM., Hum Mutat 2003;22:79-85).

In some cases, the selectivity of AS-PCR has been increased anywhere from detection of 1 in 10 alleles to 1 in 100,000 alleles by using SNP-based PCR primers containing locked nucleic acids (LNAs) (Latorra, D., et al., Hum Mut 2003, 2:79-85; Nakiandwe, J. et al., Plant Method 2007, 3:2) or modified bases (Koizumi, M. et al. Anal Biochem. 2005, 340:287-294). However, these base “mimics” or modifications increase the overall cost of analysis and often require extensive optimization.

Another technology involving probe hybridization methods used for discriminating allelic variations is TaqMan® genotyping. However, like AS-PCR, selectivity using this method is limited and not suitable for detecting rare (1 in >1,000) alleles or mutations in a mixed sample.

### SUMMARY

In some embodiments, the present inventions relates generally to compositions, methods and kits for use in discriminating sequence variation between different alleles. More specifically, in some embodiments, the present invention provides for compositions, methods and kits for quantitating rare (e.g., mutant) allelic variants, such as SNPs, or nucleotide (NT) insertions or deletions, in samples comprising abundant (e.g., wild type) allelic variants with high specificity. In particular, in some embodiments, the invention relates to a highly selective method for mutation detection referred to as competitive allele-specific TaqMan PCR (“cast-PCR”).

In one aspect, the present invention provides compositions for use in identifying and/or quantitating allelic variants in nucleic acid samples. Some of these compositions can comprise: (a) an allele-specific primer; (b) an allele-specific blocker probe; (c) a detector probe; and/or (d) a locus-specific primer.

In some embodiments of the compositions, the allele-specific primer comprises a target-specific portion and an allele-specific nucleotide portion. In some embodiments, the allele-specific primer may further comprise a tail. In some exemplary embodiments, the tail is located at the 5′ end of the allele-specific primer. In other embodiments, the tail of the allele-specific primer has repeated guanine and cytosine residues (“GC-rich”). In some embodiments, the melting temperature (“Tm”) of the entire allele-specific primer ranges from about 50° C. to 67° C. In some embodiments, the allele-specific primer concentration is between about 20-900 nM.

In some embodiments of the compositions, the allele-specific nucleotide portion of the allele-specific primer is located at the 3′ terminus. In some embodiments, the selection of the allele-specific nucleotide portion of the allele-specific primer involves the use of a highly discriminating base (e.g., for detection of A/A, A/G, G/A, G/G, A/C, or C/A alleles). In some embodiments, for example when the allele to be detected involves A/G or C/T SNPs, A or G is used as the 3′ allele-specific nucleotide portion of the allele-specific primer (e.g., if A or T is the target allele), or C or T is used as the 3′ allele-specific nucleotide portion of the allele-specific primer (e.g., if C or G is the target allele). In other embodiments, A is used as the discriminating base at the 3′ end of the allele-specific primer when detecting and/or quantifying A/T SNPs. In other embodiments, G is used as the discriminating base at the 3′ end of the allele-specific primer when detecting and/or quantifying C/G SNPs.

In some embodiments of the compositions, the allele-specific blocker probe comprises a non-extendable blocker moiety at the 3′ terminus. In some exemplary embodiments, the non-extendable blocker moiety is a minor groove binder (MGB). In some embodiments, the target allele position is located about 6-10, such as about 6, about 7, about 8, about 9, or about 10 nucleotides away from the non-extendable blocker moiety of the allele-specific blocker probe. In some embodiments, the allele-specific blocker probe comprises an MGB moiety at the 5′ terminus. In some exemplary embodiments, the allele-specific blocker probe is not cleaved during PCR amplification. In some embodiments, the Tm of the allele-specific blocker probe ranges from about 58° C. to 66° C.

In some embodiments of the compositions, the allele-specific blocker probe and/or allele-specific primer comprise at least one modified base. In some embodiments, the modified base(s) may increase the difference in the Tm between matched and mismatched target sequences and/or decrease mismatch priming efficiency, thereby improving not only assay specificity bust also selectivity. Such modified base(s) may include, for example, 8-Aza-7-deaza-dA (ppA), 8-Aza-7-deaza-dG (ppG), 2′-Deoxypseudoisocytidine (iso dC), 5-fluoro-2′-deoxyuridine (fdU), locked nucleic acid (LNA), or 2′-O,4′-C-ethylene bridged nucleic acid (ENA) bases (also see, for example, FIG. 4B). In some embodiments the modified base is located at (a) the 3′-end, (b) the 5′-end, (c) at an internal position or at any combination of (a), (b) or (c) within the allele-specific blocker probe and/or the allele-specific primer.

In some embodiments of the compositions, the detector probe is a sequence-based or locus-specific detector probe. In other embodiments the detector probe is a 5′ nuclease probe. In some exemplary embodiments, the detector probe can comprises an MGB moiety, a reporter moiety (e.g., FAM™, TET™, JOE™, VIC™, or SYBR® Green), a quencher moiety (e.g., Black Hole Quencher™ or TAMRA™;), and/or a passive reference (e.g., ROX™). In some exemplary embodiments, the detector probe is designed according to the methods and principles described in U.S. Pat. No. 6,727,356 (the disclosure of which is incorporated herein by reference in its entirety). In some exemplary embodiments, the detector probe is a TaqMan® probe (Applied Biosystems, Foster City).

In some embodiments of the compositions, the composition can further comprise a polymerase; deoxyribonucleotide triphosphates (dNTPs); other reagents and/or buffers suitable for amplification; and/or a template sequence or nucleic acid sample. In some embodiments, the polymerase can be a DNA polymerase. In some other embodiments, the polymerase can be thermostable, such as Taq DNA polymerase. In other embodiments, the template sequence or nucleic acid sample can be DNA, such as genomic DNA (gDNA) or complementary DNA (cDNA). In other embodiments the template sequence or nucleic acid sample can be RNA, such as messenger RNA (mRNA).

In another aspect, the present disclosure provides methods for amplifying an allele-specific sequence. Some of these methods can include one or more of the following: (a) hybridizing an allele-specific primer to a first nucleic acid molecule comprising a first allele (allele-1); (b) hybridizing an allele-specific blocker probe to a second nucleic acid molecule comprising a second allele (allele-2), wherein allele-2 corresponds to the same loci as allele-1; (c) hybridizing a detector probe to the first nucleic acid molecule; (d) hybridizing a locus-specific primer to the extension product of the allele-specific primer; and (e) PCR amplifying the first nucleic acid molecule comprising allele-1.

In another aspect, the present invention provides methods for detecting and/or quantitating an allelic variant in a pooled or mixed sample comprising other alleles. Some of these methods can include one or more of the following: (a) in a first reaction mixture hybridizing a first allele-specific primer to a fist nucleic acid molecule comprising a first allele (allele-1) and in a second reaction mixture hybridizing a second allele-specific primer to a first nucleic acid molecule comprising a second allele (allele-2), wherein allele-2 corresponds to the same locus as allele-1; (b) in the first reaction mixture hybridizing a first allele-specific blocker probe to a second nucleic acid molecule comprising allele-2 and in the second reaction mixture hybridizing a second allele-specific blocker probe to a second nucleic acid molecule comprising allele-1; (c) in the first reaction mixture, hybridizing a first detector probe to the first nucleic acid molecule and in the second reaction mixture and hybridizing a second detector probe to the first nucleic acid molecule; (d) in the first reaction mixture hybridizing a first locus-specific primer to the extension product of the first allele-specific primer and in the second reaction mixture hybridizing a second locus-specific primer to the extension product of the second allele-specific primer; and (e) PCR amplifying the first nucleic acid molecule to form a first set or sample of amplicons and PCR amplifying the second nucleic acid molecule to form a second set or sample of amplicons; and (f) comparing the first set of amplicons to the second set of amplicons to quantitate allele-1 in the sample comprising allele-2 and/or allele-2 in the sample comprising allele-1.

In some embodiments of the methods, the first and/or second allele-specific primer comprises a target-specific portion and an allele-specific nucleotide portion. In some embodiments, the first and/or second allele-specific primer may further comprise a tail. In some embodiments, the Tm of the entire first and/or second allele-specific primer ranges from about 50° C. to 67° C. In some embodiments the first and/or second allele-specific primer concentration is between about 20-900 nM.

In some embodiments of the methods, the target-specific portion of the first allele-specific primer and the target-specific portion of the second allele-specific primer comprise the same sequence. In other embodiments, the target-specific portion of the first allele-specific primer and the target-specific portion of the second allele-specific primer are the same sequence.

In some embodiments of the methods, the tail is located at the 5′-end of the first and/or second allele-specific primer. In some embodiments, the 5′ tail of the first allele-specific primer and the 5′ tail of the second allele-specific primer comprise the same sequence. In other embodiments, the 5′ tail of the first allele-specific primer and the 5′ tail of the second allele-specific primer are the same sequence. In other embodiments, the tail of the first and/or second allele-specific primer is GC-rich.

In some embodiments of the methods, the allele-specific nucleotide portion of the first allele-specific primer is specific to a first allele (allele-1) of a SNP and the allele-specific nucleotide portion of the second allele-specific primer is specific to a second allele (allele-2) of the same SNP. In some embodiments of the methods, the allele-specific nucleotide portion of the first and/or second allele-specific primer is located at the 3′-terminus. In some embodiments, the selection of the allele-specific nucleotide portion of the first and/or second allele-specific primer involves the use of a highly discriminating base (e.g., for detection of A/A, A/G, G/A, G/G, A/C, or C/A alleles). In some embodiments, for example when the allele to be detected involves A/G or C/T SNPs, A or G is used as the 3′ allele-specific nucleotide portion of the first and/or second allele-specific primer (e.g., if A or T is the major allele), or C or T is used as the 3′ allele-specific nucleotide portion of the first and/or second allele-specific primer (e.g., if C or G is the major allele). In other embodiments, A is used as the discriminating base at the 3′ end of the first and/or second allele-specific primer when detecting and/or quantifying A/T SNPs. In other embodiments, G is used as the discriminating base at the 3′ end of the first and/or second allele-specific primer when detecting and/or quantifying C/G SNPs.

In some embodiments of the methods, the first and/or second allele-specific blocker probe comprises a non-extendable blocker moiety at the 3′ terminus. In some exemplary embodiments, the non-extendable blocker moiety is an MGB. In some embodiments, the target allele position is located about 6-10, such as about 6, about 7, about 8, about 9, or about 10 nucleotides away from the non-extendable blocker moiety of the first and/or second allele-specific blocker probe. In some embodiments, the first and/or second allele-specific blocker probe comprises an MGB moiety at the 5′-terminus. In other embodiments, the first and/or second allele-specific blocker probe is not cleaved during PCR amplification. In some embodiments, the Tm of the first and/or second allele-specific blocker probe ranges from about 58° C. to 66° C.

In some embodiments of the methods, the first and/or second allele-specific blocker probe and/or the first and/or second allele-specific primer comprises at least one modified base. In some embodiments, the modified base(s) may increase the difference in the Tm between matched and mismatched target sequences and/or decrease mismatch priming efficiency, thereby improving not only assay specificity, but also selectivity. Such modified base(s) may include, for example, 8-Aza-7-deaza-dA (ppA), 8-Aza-7-deaza-dG (ppG), 2′-Deoxypseudoisocytidine (iso dC), 5-fluoro-2′-deoxyuridine (fdU), locked nucleic acid (LNA), or 2′-O,4′-C-ethylene bridged nucleic acid (ENA) bases (see also, for example, FIG. 4B). In some embodiments the modified base is located at (a) the 3′-end, (b) the 5′-end, (c) at an internal position or at any combination of (a), (b) or (c) within said first and/or second allele-specific blocker probe and/or the first and/or second allele-specific primer.

In some embodiments of the methods, the specificity of allelic discrimination is improved by the inclusion of a modified base in the first and/or second allele-specific primer and/or first, and/or second allele-specific blocker probe as compared to the use of a non-modified allelic-specific primer or blocker probe. In some embodiments, the improvement in specificity is at least 2 fold.

In some embodiments of the methods, the specificity of allelic discrimination is at least 2 fold better than the specificity of allelic discrimination using Allele-Specific PCR with a Blocking reagent (ASB-PCR) methods.

In some embodiments, the methods further comprise a 2-stage cycling protocol. In some embodiments, the number of cycles in the first stage of the 2-stage cycling protocol comprises fewer cycles than the number of cycles used in the second stage. In other embodiments, the number of cycles in the first stage is about 90% fewer cycles than the number of cycles in the second stage. In yet other embodiments, the number of cycles in the first stage is between 3-7 cycles and the number of cycles in the second stage is between 42-48 cycles.

In some embodiments, the annealing/extension temperature used during the first cycling stage of the 2-stage cycling protocol is between 1-3° C. lower than the annealing/extension temperature used during the second stage. In preferred embodiments, the annealing/extension temperature used during the first cycling stage of the 2-stage cycling protocol is between 56-59° C. and the annealing/extension temperature used during the second stage is between 60-62° C.

In some embodiments, the methods further comprise a pre-amplification step. In preferred embodiments, the pre-amplification step comprises a multiplex amplification reaction that uses at least two complete sets of allele-specific primers and locus-specific primers, wherein each set is suitable or operative for amplifying a specific polynucleotide of interest. In other embodiments, the products of the multiplex amplification reaction are divided into secondary single-plex amplification reactions, such as a cast-PCR reaction, wherein each single-plex reaction contains at least one primer set previously used in the multiplex reaction. In other embodiments, the multiplex amplification reaction further comprises a plurality of allele-specific blocker probes. In some embodiments, the multiplex amplification reaction is carried out for a number of cycles suitable to keep the reaction within the linear phase of amplification.

In some embodiments of the methods, the first and/or second detector probes are the same. In some embodiments, the first and/or second detector probes are different. In some embodiments, the first and/or second detector probe is a sequence-based or locus-specific detector probe. In other embodiments the first and/or second detector probe is a 5′ nuclease probe. In some exemplary embodiments, the first and/or second detector probes comprises an MGB moiety, a reporter moiety (e.g., FAM™, TET™, JOE™, VIC™, or SYBR® Green), a quencher moiety (e.g., Black Hole Quencher™ or TAMRA™;), and/or a passive reference (e.g., ROX™). In some exemplary embodiments, the first and/or second detector probe is designed according to the methods and principles described in U.S. Pat. No. 6,727,356 (the disclosure of which is incorporated herein by reference in its entirety). In some exemplary embodiments, the detector probe is a TaqMan® probe.

In some embodiments of the methods, the first locus-specific primer and the second locus-specific primer comprise the same sequence. In some embodiments the first locus-specific primer and the second locus-specific primer are the same sequence.

In some embodiments of the methods, the first and/or second reaction mixtures can further comprises a polymerase; dNTPs; other reagents and/or buffers suitable for PCR amplification; and/or a template sequence or nucleic acid sample. In some embodiments, the polymerase can be a DNA polymerase. In some embodiments, the polymerase can be thermostable, such as Taq DNA polymerase. In some embodiments, the template sequence or nucleic acid sample can be DNA, such as gDNA or cDNA. In other embodiments the template sequence or nucleic acid sample can be RNA, such as mRNA.

In some embodiments of the methods, the first allele-specific blocker probe binds to the same strand or sequence as the second allele-specific primer, while the second allele-specific blocker probe binds to the same strand or sequence as the first allele-specific primer. In some embodiments, the first and/or second allele-specific blocker probes are used to reduce the amount of background signal generated from either the second allele and/or the first allele, respectively. In some embodiments, first and/or second allele-specific blocker probes are non-extendable and preferentially anneal to either the second allele or the first allele, respectively, thereby blocking the annealing of, for example, the extendable first allele-specific primer to the second allele and/or the extendable second allele-specific primer to first allele.

In some exemplary embodiments, the first allele is a rare (e.g., minor) or mutant allele. In other exemplary embodiments the second allele is an abundant (e.g., major) or wild type allele.

In another aspect, the present invention provides kits for quantitating a first allelic variant in a sample comprising a second allelic variant involving: (a) a first allele-specific primer; (b) a second allele-specific primer; (c), a first locus-specific primer; (d) a second locus-specific primer; (e) a first allele-specific blocker probe; (f) a second allele-specific blocker probe; and (g) a first locus-specific detector probe and (h) a second locus-specific detector probe.

In some embodiments of the kits, the first and/or second allele-specific primer comprises a target-specific portion and an allele-specific nucleotide portion. In some embodiments, the first and/or second allele-specific primer may further comprise a tail. In some embodiments, the Tm of the entire first and/or second allele-specific primer ranges from about 50° C. to 67° C. In some embodiments the first and/or second allele-specific primer concentrations are between about 20-900 nM.

In some embodiments of the kits, the target-specific portion of the first allele-specific primer and the target-specific portion of the second allele-specific primer comprise the same sequence. In other embodiments, the target-specific portion of the first allele-specific primer and the target-specific portion of the second allele-specific primer are the same sequence.

In some embodiments of the kits, the tail is located at the 5′ end of the first and/or second allele-specific primer. In some embodiments, the 5′ tail of the first allele-specific primer and the 5′ tail of the second allele-specific primer comprise the same sequence. In other embodiments, the 5′ tail of the first allele-specific primer and the 5′ tail of the second allele-specific primer are the same sequence. In other embodiments, the tail of the first and/or second allele-specific primer is GC rich.

In some embodiments of the kits, the allele-specific nucleotide portion of the first allele-specific primer is specific to a first allele (allele-1) of a SNP and the allele-specific nucleotide portion of the second allele-specific primer is specific to a second allele (allele-2) of the same SNP. In some embodiments of the disclosed methods, the allele-specific nucleotide portion of the first and/or second allele-specific primer is located at the 3′ terminus. In some embodiments, the selection of the allele-specific nucleotide portion of the first and/or second allele-specific primer involves the use of a highly discriminating base (e.g., for detection of A/A, A/G, G/A, G/G, A/C, or C/A alleles) (FIG. 2). In some embodiments, for example when the allele to be detected involves A/G or C/T SNPs, A or G is used as the 3′ allele-specific nucleotide portion of the first and/or second allele-specific primer (e.g., if A or T is the major allele), or C or T is used as the 3′ allele-specific nucleotide portion of the first and/or second allele-specific primer (e.g., if C or G is the major allele). In other embodiments, A is used as the discriminating base at the 3′ end of the first and/or second allele-specific primer when detecting and/or quantifying A/T SNPs. In other embodiments, G is used as the discriminating base at the 3′ end of the first and/or second allele-specific primer when detecting and/or quantifying C/G SNP s.

In some embodiments of the kits, the first and/or second allele-specific blocker probe comprises a non-extendable blocker moiety at the 3′ terminus. In some exemplary embodiments, the non-extendable blocker moiety is an MGB. In some embodiments, the target allele position is located about 6-10, such as about 6, about 7, about 8, about 9, or about 10 nucleotides away from the non-extendable blocker moiety of the first and/or second allele-specific blocker probe. In some embodiments, the first and/or second allele-specific blocker probe comprises an MGB moiety at the 5′ terminus. In other embodiments, the first and/or second allele-specific blocker probe is not cleaved during PCR amplification. In some embodiments, the Tm of the first and/or second allele-specific blocker probe ranges from about 58° C. to 66° C.

In some embodiments of the kits, the allele-specific blocker probe and/or the first and/or second allele-specific primer comprises at least one modified base. In some embodiments, the modified base(s) may increase the difference in the Tm between matched and mismatched target sequences and/or decrease mismatch priming efficiency, thereby improving not only assay specificity bust also selectivity. Such modified base(s) may include, for example, 8-Aza-7-deaza-dA (ppA), 8-Aza-7-deaza-dG (ppG), 2′-Deoxypseudoisocytidine (iso dC), 5-fluoro-2′-deoxyuridine (fdU), locked nucleic acid (LNA), or 2′-O,4′-C-ethylene bridged nucleic acid (ENA) bases (see also, for example, FIG. 4B). In some embodiments the modified base is located at (a) the 3′-end, (b) the 5′-end, (c) at an internal position or at any combination of (a), (b) or (c) within said first and/or second allele-specific blocker probe and/or the first and/or second allele-specific primer.

In some embodiments of the kits, the first and/or second detector probes are the same. In some embodiments of the disclosed kits the first and/or second detector probes are different. In some embodiments of the disclosed kits, the first and/or second detector probes are sequence-based or locus-specific detector probes. In other embodiments the first and/or second detector probe are 5′ nuclease probes. In some exemplary embodiments, the first and/or second detector probes comprise an MGB moiety, a reporter moiety (e.g., FAM™, TET™, JOE™, VIC™, or SYBR® Green), a quencher moiety (e.g., Black Hole Quencher™ or TAMRA™;), and/or a passive reference (e.g., ROX™). In some exemplary embodiments, the first and/or second detector probe are designed according to the methods and principles described in U.S. Pat. No. 6,727,356 (the disclosure of which is incorporated herein by reference in its entirety). In some exemplary embodiments, the detector probe is a TaqMan® probe.

In some embodiments of the kits, the first locus-specific primer and the second locus-specific primer comprise the same sequence. In some embodiments the first locus-specific primer and the second locus-specific primer are the same sequence.

In some embodiments of the kits, the first and/or second reaction mixture can further comprise a polymerase; dNTPs; other reagents and/or buffers suitable for PCR amplification; and/or a template sequence or nucleic acid sample. In some embodiments, the polymerase can be a DNA polymerase. In some other embodiments, the polymerase can be thermostable, such as Taq DNA polymerase.

In some embodiments, the compositions, methods and kits of the present invention provide high allelic discrimination specificity and selectivity. In some embodiments, the quantitative determination of specificity and/or selectivity comprises a comparison of Ct values between a first set of amplicons and a second set of amplicons. In some embodiments, selectivity is at a level whereby a single copy of a given allele in about 1 million copies of another allele or alleles can be detected.

The foregoing has described various embodiments of the invention that provide improved detection and discrimination of allelic variants using one or more of the following: (a) tailed allele-specific primers; (b) low allele-specific primer concentration; (c) allele-specific primers designed to have lower Tms; (d) allele-specific primers designed to target discriminating bases; (e) allele-specific blocker probes containing MGB, designed to prevent amplification from alternative, and potentially more abundant, allelic variants in a sample; and (f) allele-specific blocker probes and/or allele-specific primers designed to comprise modified bases in order to increase the delta Tm between matched and mismatched target sequences.

While particular embodiments employing several of the above improvements have been discussed herein, it will be apparent to the skilled artisan that depending on the nature of the sample to be examined, various combinations of the above improvements can be combined to arrive at a favorable result. Thus, for example, non-MGB blocker probes can be used with an embodiment that include methods employing allele-specific primers containing modified bases to increase delta Tm; such primers can also be designed to target discriminating bases; and the primers can be used at low primer concentrations. Accordingly, alternative embodiments based upon the present disclosure can be used to achieve a suitable level of allelic detection.

The present disclosure provides the advantage that any of the combinations of listed improvements could be utilized by a skilled artisan in a particular situation. For example, the current invention can include a method or reaction mixture that employs improvements a, c, d and f; improvements b, c, and e; or improvements

It is to be understood that both the foregoing general description and the following detailed description are exemplary and explanatory only and are not restrictive of the invention, as claimed.

The accompanying drawings, which are incorporated in and constitute a part of this specification, illustrate several exemplary embodiments of the disclosure and together with the description, serve to explain certain teachings.

### BRIEF DESCRIPTION OF THE DRAWINGS

The skilled artisan will understand that the drawings described below are for illustration purposes only. The drawings are not intended to limit the scope of the present teachings in any way.

FIG. 1 depicts a schematic of an illustrative embodiment of cast-PCR. In some embodiments, components of cast-PCR include the following: one locus-specific TaqMan probe (LST); two MGB blockers: one allele-1-specific MGB blocker (MGB 1) and one allele-2-specific MGB blocker (MGB2); 3 PCR primers: one locus-specific PCR primer (LSP); one allele-1-specific primer (ASP1) and one allele-2-specific primer (ASP2).

FIG. 2 depicts a schematic of an illustrative embodiment of cast-PCR using allele-specific blocker probes comprising highly discriminating bases for detecting rare allelic variants. Highly discriminating bases may include, for example, A/A, A/G, G/A, G/G, A/C, C/A. The least discriminating bases may include, for example, C/C, T/C, G/T, T/G, C/T. In some embodiments, for example, for detection of A-G or C-T SNPs, A & G are used as the discriminating base if A//T is allelic variant (e.g., mutant allele); or C & T are used as the discriminating base if C//G allelic variant (e.g., mutant allele).

FIG. 3 depicts a schematic of an illustrative embodiment of cast-PCR using an allele-specific blocker probe with an MGB moiety at the 5′ end. In some embodiments the blocker moiety at the 3′-end of the probe may include, for example, NH₂, biotin, MGB, PO₄, and PEG.

FIG. 4A depicts a schematic of an illustrative embodiment of cast-PCR using modified bases in an MGB blocker probe or allele-specific primer. (G* represents ppG.)

FIG. 4B depicts some examples of modified bases of an MGB blocker probe or allele-specific primer.

FIG. 5 depicts the TaqMan-like sensitivity and dynamic range of one exemplary embodiment of cast-PCR.

FIG. 6 depicts the sequence of KRAS mutations at codons 12 and 13 that are detectable using cast-PCR methods. KRAS mutations at codons 12 and 13 are associated with resistance to cetuxima or panitumumab in metastatic colorectal cancer (Di Nicolantonio F., et al., J Clin Oncol. 2008; 26:5705-12). FIG. 6 discloses SEQ ID NO: 79.

FIG. 7 depicts the specificity of KRAS mutation detection using cast-PCR assays in one exemplary embodiment.

FIG. 8 depicts one exemplary embodiment using cast-PCR methods to detect a single copy of mutant DNA in 10⁶ copies of wild-type DNA.

FIG. 9 depicts detection of the relative copy number of mutant samples (KRAS-G12A) spiked in wild type samples using cast-PCR methods.

FIG. 10 depicts a number of different tumor markers (SNPs) detected in tumor samples using one exemplary embodiment of cast-PCR.

FIG. 11A-E shows a list of exemplary primers and probes used in cast-PCR assays. Nucleotides shown in lower case are the tailed portion of the primers. The nucleotide-portion of allele-specific primers (ASP) is at the 3′-most terminus of each primer and are indicated in bold. The allele positions of the blocker probes (MGB) are located at various internal positions relative to the blocker moieties, in some cases, are indicated in bold. FIG. 11A discloses ‘ASP 1’ as SEQ ID NOS 80-84, respectively, in order of appearance, ‘LSP’ as SEQ ID NOS 85-89, respectively, in order of appearance, ‘MGB1’ as SEQ ID NOS 90-94, respectively, in order of appearance, ‘ASP2’ as SEQ ID NOS 95-99, respectively, in order of appearance, ‘LST’ as SEQ ID NOS 100-104, respectively, in order of appearance, and ‘MGB2’ as SEQ ID NOS 105-109, respectively, in order of appearance. FIG. 11B discloses ‘ASP 1’ as SEQ ID NOS 110-135, respectively, in order of appearance, and ‘ASP2’ as SEQ ID NOS 136-161, respectively, in order of appearance. FIG. 11C discloses ‘ASP1’ as SEQ ID NOS 162-187, respectively, in order of appearance, and ‘ASP2’ as SEQ ID NOS 188-213, respectively, in order of appearance. FIG. 11D discloses ‘LSP’ as SEQ ID NOS 214-239, respectively, in order of appearance, and ‘LST’ as SEQ ID NOS 240-265, respectively, in order of appearance. FIG. 11E discloses ‘MGB1’ as SEQ ID NOS 266-292, respectively, in order of appearance, ‘MGB2’ as SEQ ID NOS 293-317, respectively, in order of appearance.

FIG. 12 depicts, in one exemplary embodiment, the specificity of allelic discrimination for samples that were pre-amplified prior to analysis by cast-PCR.

FIG. 13 depicts, in on exemplary embodiment, the specificity of allelic discrimination for cast-PCR assays performed using tailed versus non-tailed allele-specific primers.

FIG. 14 depicts, in on exemplary embodiment, the specificity of allelic discrimination for samples analyzed by cast-PCR versus samples analyzed by ASB-PCR methods.

FIG. 15 depicts, in on exemplary embodiment, the specificity of allelic discrimination for cast-PCR assays performed using MGB blocker probes or phosphate blocker probes.

FIG. 16 depicts, in one exemplary embodiment, the specificity of allelic discrimination for cast-PCR assays performed using LNA-modified allele-specific primers.

FIG. 17 compares, in one exemplary embodiment, the specificity of allelic discrimination for cast-PCR assays performed using various chemically-modified allele-specific primers.

FIGS. 18A and 18B show a list of exemplary allele-specific primers and probes used in pre-amplification and cast-PCR assays. FIG. 18A discloses ‘ASP1’ as SEQ ID NOS 318-324, respectively, in order of appearance, ‘LSP’ as SEQ ID NOS 325-331, respectively, in order of appearance, ‘ASP2’ as SEQ ID NOS 332-338, respectively, in order of appearance, and ‘LST’ as SEQ ID NOS 339-345, respectively, in order of appearance. FIG. 18B discloses ‘MGB1’ as SEQ ID NOS 346-352, respectively, in order of appearance, and ‘MGB2’ as SEQ ID NOS 353-359, respectively, in order of appearance.

FIG. 19A-D shows a list of exemplary primers and probes used in cast-PCR assays using either tailed (ASP+tail) or non-tailed (ASP−tail) allele-specific primers. (The tailed portion of the ASP+tail primers are indicated in bold.). FIG. 19A discloses ‘ASP1-tail’ as SEQ ID NOS 360-371, respectively, in order of appearance, and ‘ASP2-tail’ as SEQ ID NOS 372-383, respectively, in order of appearance. FIG. 19B discloses ‘ASP1+tail’ as SEQ ID NOS 384-395, respectively, in order of appearance, and ‘ASP2+tail’ as SEQ ID NOS 396-407, respectively, in order of appearance. FIG. 19C discloses ‘LSP’ as SEQ ID NOS 408-419, respectively, in order of appearance, and ‘LST’ as SEQ ID NOS 420-431, respectively, in order of appearance. FIG. 19D discloses ‘MGB1’ as SEQ ID NOS 432-443, respectively, in order of appearance, and ‘MGB2’ as SEQ ID NOS 444-455, respectively, in order of appearance.

FIG. 20A-C shows a list of exemplary primers and probes used in ASB-PCR. The blocker probes used in ASB-PCR comprise a phosphate group at the 3′-end of the blocker probes (PHOS). FIG. 20A discloses ‘ASP 1’ as SEQ ID NOS 456-467, respectively, in order of appearance, and ‘ASP2’ as SEQ ID NOS 468-479, respectively, in order of appearance. FIG. 20B discloses ‘LSP’ as SEQ ID NOS 480-491, respectively, in order of appearance, and ‘LST’ as SEQ ID NOS 492-503, respectively, in order of appearance. FIG. 20C discloses ‘PHOS1’ as SEQ ID NOS 504-515, respectively, in order of appearance, and ‘PHOS2’ as SEQ ID NOS 516-527, respectively, in order of appearance.

FIG. 21A-C shows a list of exemplary primers and probes used in cast-PCR assays performed using LNA-modified allele-specific primers. In this exemplary embodiment, the LNA modifications of the ASP are at the 3′-ends. (“+” indicates the LNA modified nucleotide and are notated in parentheses.) FIG. 21A discloses ‘ASP1’ as SEQ ID NOS 528-539, respectively, in order of appearance, and ‘ASP2’ as SEQ ID NOS 540-551, respectively, in order of appearance. FIG. 21B discloses ‘LSP’ as SEQ ID NOS 552-563, and ‘LST’ as SEQ ID NOS 564-575, respectively, in order of appearance. FIG. 21C discloses ‘MGB1’ as SEQ ID NOS 576-587, respectively, in order of appearance, and ‘MGB2’ as SEQ ID NOS 588-599, respectively, in order of appearance.

FIG. 22 shows a list of exemplary primers and probes used in cast-PCR assays performed using chemically modified allele-specific primers. In this exemplary embodiment, the chemical modifications (e.g., ppA, ppG, fdU, and iso dC) of the ASP are at the 3′-ends. (The chemically modified nucleotides are shown in parentheses.) FIG. 22 discloses ‘ASP1’ as SEQ ID NOS 600-605, respectively, in order of appearance, ‘LSP’ as SEQ ID NOS 606-611, respectively, in order of appearance, ‘MGB1’ as SEQ ID NOS 612-617, respectively, in order of appearance, ‘ASP2’ as SEQ ID NOS 618-623, respectively, in order of appearance, ‘LST’ as SEQ ID NOS 624-629, respectively, in order of appearance, and ‘MGB2’ as SEQ ID NOS 630-635, respectively, in order of appearance.

### DETAILED DESCRIPTION

### I. Introduction

The selective amplification of an allele of interest is often complicated by factors including the mispriming and extension of a mismatched allele-specific primer on an alternative allele. Such mispriming and extension can be especially problematic in the detection of rare alleles present in a sample populated by an excess of another allelic variant. When in sufficient excess, the mispriming and extension of the other allelic variant may obscure the detection of the allele of interest. When using PCR-based methods, the discrimination of a particular allele in a sample containing alternative allelic variants relies on the selective amplification of an allele of interest, while minimizing or preventing amplification of other alleles present in the sample.

A number of factors have been identified, which alone or in combination, contribute to the enhanced discriminating power of allele-specific PCR. As disclosed herein, a factor which provides a greater ΔCt value between a mismatched and matched allele-specific primer is indicative of greater discriminating power between allelic variants. Such factors found to improve discrimination of allelic variants using the present methods include, for example, the use of one or more of the following: (a) tailed allele-specific primers; (b) low allele-specific primer concentration; (c) allele-specific primers designed to have lower Tms; (d) allele-specific primers designed to target discriminating bases; (e) allele-specific blocker probes designed to prevent amplification from alternative, and potentially more abundant, allelic variants in a sample; and (f) allele-specific blocker probes and/or allele-specific primers designed to comprise modified bases in order to increase the delta Tm between matched and mismatched target sequences.

The above-mentioned factors, especially when used in combination, can influence the ability of allele-specific PCR to discriminate between different alleles present in a sample. Thus, the present disclosure relates generally to novel amplification methods referred to as cast-PCR, which utilizes a combination of factors referred to above to improve discrimination of allelic variants during PCR by increasing ΔCt values. In some embodiments, the present methods can involve high levels of selectivity, wherein one mutant molecule in a background of at least 1,000 to 1,000,000, such as about 1000-10,000, about 10,000 to 100,000, or about 100,000 to 1,000,000 wild type molecules, or any fractional ranges in between can be detected. In some embodiments, the comparison of a first set of amplicons and a second set of amplicons involving the disclosed methods provides improvements in specificity from 1,000× to 100,000,000× fold difference, such as about 1000-10,000×, about 10,000 to 100,000×, about 100,000 to 1,000,000× or about 1,000,000 to 100,000,000× fold difference, or any fractional ranges in between.

### II. Definitions

For the purposes of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended.

As used herein, the term “allele” refers generally to alternative DNA sequences at the same physical locus on a segment of DNA, such as, for example, on homologous chromosomes. An allele can refer to DNA sequences which differ between the same physical locus found on homologous chromosomes within a single cell or organism or which differ at the same physical locus in multiple cells or organisms (“allelelic variant”). In some instances, an allele can correspond to a single nucleotide difference at a particular physical locus. In other embodiments and allele can correspond to nucleotide (single or multiple) insertion or deletion.

As used herein, the term “allele-specific primer” refers to an oligonucleotide sequence that hybridizes to a sequence comprising an allele of interest, and which when used in PCR can be extended to effectuate first strand cDNA synthesis. Allele-specific primers are specific for a particular allele of a given target DNA or loci and can be designed to detect a difference of as little as one nucleotide in the target sequence. Allele-specific primers may comprise an allele-specific nucleotide portion, a target-specific portion, and/or a tail.

As used herein, the terms “allele-specific nucleotide portion” or “allele-specific target nucleotide” refers to a nucleotide or nucleotides in an allele-specific primer that can selectively hybridize and be extended from one allele (for example, a minor or mutant allele) at a given locus to the exclusion of the other (for example, the corresponding major or wild type allele) at the same locus.

As used herein, the term “target-specific portion” refers to the region of an allele-specific primer that hybridizes to a target polynucleotide sequence. In some embodiments, the target-specific portion of the allele-specific primer is the priming segment that is complementary to the target sequence at a priming region 5′ of the allelic variant to be detected. The target-specific portion of the allele-specific primer may comprise the allele-specific nucleotide portion. In other instances, the target-specific portion of the allele-specific primer is adjacent to the 3′ allele-specific nucleotide portion.

As used herein, the terms “tail” or “5′-tail” refers to the non-3′ end of a primer. This region typically will, although does not have to contain a sequence that is not complementary to the target polynucleotide sequence to be analyzed. The 5′ tail can be any of about 2-30, 2-5, 4-6, 5-8, 6-12, 7-15, 10-20, 15-25 or 20-30 nucleotides, or any range in between, in length.

As used herein, the term “allele-specific blocker probe” (also referred to herein as “blocker probe,” “blocker,”) refers to an oligonucleotide sequence that binds to a strand of DNA comprising a particular allelic variant which is located on the same, opposite or complementary strand as that bound by an allelic-specific primer, and reduces or prevents amplification of that particular allelic variant. As discussed in greater detail herein, allele-specific blocker probes generally comprise modifications, e.g., at the 3′-OH of the ribose ring, which prevent primer extension by a polymerase. The allele-specific blocker probe can be designed to anneal to the same or opposing strand of what the allele-specific primer anneals to and can be modified with a blocking group (e.g., a “non-extendable blocker moiety”) at its 3′ terminal end. Thus, a blocker probe can be designed, for example, so as to tightly bind to a wild type allele (e.g., abundant allelic variant) in order to suppress amplification of the wild type allele while amplification is allowed to occur on the same or opposing strand comprising a mutant allele (e.g., rare allelic variant) by extension of an allele-specific primer. In illustrative examples, the allele-specific blocker probes do not include a label, such as a fluorescent, radioactive, or chemiluminescent label

As used herein, the term “non-extendable blocker moiety” refers generally to a modification on an oligonucleotide sequence such as a probe and/or primer which renders it incapable of extension by a polymerase, for example, when hybridized to its complementary sequence in a PCR reaction. Common examples of blocker moieties include modifications of the ribose ring 3′-OH of the oligonucleotide, which prevents addition of further bases to the ‘3-end of the oligonucleotide sequence a polymerase. Such 3’-OH modifications are well known in the art. (See, e.g., Josefsen, M., et al., Molecular and Cellular Probes, 23 (2009):201-223; McKinzie, P. et al., Mutagenesis. 2006, 21(6):391-7; Parsons, B. et al., Methods Mol Biol. 2005, 291:235-45; Parsons, B. et al., Nucleic Acids Res. 1992, 25:20(10):2493-6; and Morlan, J. et al., PLoS One 2009, 4 (2): e4584, the disclosures of which are incorporated herein by reference in their entireties.)

As used herein, the terms “MGB,” “MGB group,” “MGB compound,” or “MBG moiety” refers to a minor groove binder. When conjugated to the 3′ end of an oligonucleotide, an MGB group can function as a non-extendable blocker moiety.

An MGB is a molecule that binds within the minor groove of double stranded DNA. Although a general chemical formula for all known MGB compounds cannot be provided because such compounds have widely varying chemical structures, compounds which are capable of binding in the minor groove of DNA, generally speaking, have a crescent shape three dimensional structure. Most MGB moieties have a strong preference for A-T (adenine and thymine) rich regions of the B form of double stranded DNA. Nevertheless, MGB compounds which would show preference to C-G (cytosine and guanine) rich regions are also theoretically possible. Therefore, oligonucleotides comprising a radical or moiety derived from minor groove binder molecules having preference for C-G regions are also within the scope of the present invention.

Some MGBs are capable of binding within the minor groove of double stranded DNA with an association constant of 10³ M⁻¹ or greater. This type of binding can be detected by well established spectrophotometric methods such as ultraviolet (UV) and nuclear magnetic resonance (NMR) spectroscopy and also by gel electrophoresis. Shifts in UV spectra upon binding of a minor groove binder molecule and NMR spectroscopy utilizing the “Nuclear Overhauser” (NOSEY) effect are particularly well known and useful techniques for this purpose. Gel electrophoresis detects binding of an MGB to double stranded DNA or fragment thereof, because upon such binding the mobility of the double stranded DNA changes.

A variety of suitable minor groove binders have been described in the literature. See, for example, Kutyavin, et al. U.S. Pat. No. 5,801,155; Wemmer, D. E., and Dervan P. B., Current Opinion in Structural Biology, 7:355-361 (1997); Walker, W. L., Kopka, J. L. and Goodsell, D. S., Biopolymers, 44:323-334 (1997); Zimmer, C.& Wahnert, U. Prog. Biophys. Molec. Bio. 47:31-112 (1986) and Reddy, B. S. P., Dondhi, S. M., and Lown, J. W., Pharmacol. Therap., 84:1-111 (1999) (the disclosures of which are herein incorporated by reference in their entireties). A preferred MGB in accordance with the present disclosure is DPI₃. Synthesis methods and/or sources for such MGBs are also well known in the art. (See, e.g., U.S. Pat. Nos. 5,801,155; 6,492,346; 6,084,102; and 6,727,356, the disclosures of which are incorporated herein by reference in their entireties.)

As used herein, the term “MGB blocker probe,” “MBG blocker,” or “MGB probe” is an oligonucleotide sequence and/or probe further attached to a minor groove binder moiety at its 3′ and/or 5′ end. Oligonucleotides conjugated to MGB moieties form extremely stable duplexes with single-stranded and double-stranded DNA targets, thus allowing shorter probes to be used for hybridization based assays. In comparison to unmodified DNA, MGB probes have higher melting temperatures (Tm) and increased specificity, especially when a mismatch is near the MGB region of the hybridized duplex. (See, e.g., Kutyavin, I. V., et al., Nucleic Acids Research, 2000, Vol. 28, No. 2: 655-661).

As used herein, the term “modified base” refers generally to any modification of a base or the chemical linkage of a base in a nucleic acid that differs in structure from that found in a naturally occurring nucleic acid. Such modifications can include changes in the chemical structures of bases or in the chemical linkage of a base in a nucleic acid, or in the backbone structure of the nucleic acid. (See, e.g., Latorra, D. et al., Hum Mut 2003, 2:79-85. Nakiandwe, J. et al., Plant Method 2007, 3:2.)

As used herein, the term “detector probe” refers to any of a variety of signaling molecules indicative of amplification. For example, SYBR® Green and other DNA-binding dyes are detector probes. Some detector probes can be sequence-based (also referred to herein as “locus-specific detector probe”), for example 5′ nuclease probes. Various detector probes are known in the art, for example (TaqMan® probes described herein (See also U.S. Pat. No. 5,538,848) various stem-loop molecular beacons (See, e.g., U.S. Pat. Nos. 6,103,476 and 5,925,517 and Tyagi and Kramer, 1996, Nature Biotechnology 14:303-308), stemless or linear beacons (See, e.g., WO 99/21881), PNA Molecular Beacons™ (See, e.g., U.S. Pat. Nos. 6,355,421 and 6,593,091), linear PNA beacons (See, e.g., Kubista et al., 2001, SPIE 4264:53-58), non-FRET probes (See, e.g., U.S. Pat. No. 6,150,097), Sunrise®/Amplifluor® probes (U.S. Pat. No. 6,548,250), stem-loop and duplex Scorpion™ probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Detector probes can comprise reporter dyes such as, for example, 6-carboxyfluorescein (6-FAM) or tetrachlorofluorescin (TET). Detector probes can also comprise quencher moieties such as tetramethylrhodamine (TAMRA), Black Hole Quenchers (Biosearch), Iowa Black (IDT), QSY quencher (Molecular Probes), and Dabsyl and Dabcel sulfonate/carboxylate Quenchers (Epoch). Detector probes can also comprise two probes, wherein for example a fluor is on one probe, and a quencher on the other, wherein hybridization of the two probes together on a target quenches the signal, or wherein hybridization on a target alters the signal signature via a change in fluorescence. Detector probes can also comprise sulfonate derivatives of fluorescein dyes with SO3 instead of the carboxylate group, phosphoramidite forms of fluorescein, phosphoramidite forms of CY5 (available, for example, from Amersham Biosciences-GE Healthcare).

As used herein, the term “locus-specific primer” refers to an oligonucleotide sequence that hybridizes to products derived from the extension of a first primer (such as an allele-specific primer) in a PCR reaction, and which can effectuate second strand cDNA synthesis of said product. Accordingly, in some embodiments, the allele-specific primer serves as a forward PCR primer and the locus-specific primer serves as a reverse PCR primer, or vice versa. In some preferred embodiments, locus-specific primers are present at a higher concentration as compared to the allele-specific primers.

As used herein, the term “rare allelic variant” refers to a target polynucleotide present at a lower level in a sample as compared to an alternative allelic variant. The rare allelic variant may also be referred to as a “minor allelic variant” and/or a “mutant allelic variant.” For instance, the rare allelic variant may be found at a frequency less than 1/10, 1/100, 1/1,000, 1/10,000, 1/100,000, 1/1,000,000, 1/10,000,000, 1/100,000,000 or 1/1,000,000,000 compared to another allelic variant for a given SNP or gene. Alternatively, the rare allelic variant can be, for example, less than 2, 3, 4, 5, 6, 7, 8, 9, 10, 15, 20, 25, 50, 75, 100, 250, 500, 750, 1,000, 2,500, 5,000, 7,500, 10,000, 25,000, 50,000, 75,000, 100,000, 250,000, 500,000, 750,000, or 1,000,000 copies per 1, 10, 100, 1,000 micro liters of a sample or a reaction volume.

As used herein, the terms “abundant allelic variant” may refer to a target polynucleotide present at a higher level in a sample as compared to an alternative allelic variant. The abundant allelic variant may also be referred to as a “major allelic variant” and/or a “wild type allelic variant.” For instance, the abundant allelic variant may be found at a frequency greater than 10×, 100×, 1,000×, 10,000×, 100,000×, 1,000,000×, 10,000,000×, 100,000,000× or 1,000,000,000× compared to another allelic variant for a given SNP or gene. Alternatively, the abundant allelic variant can be, for example, greater than 2, 3, 4, 5, 6, 7, 8, 9, 10, 15, 20, 25, 50, 75, 100, 250, 500, 750, 1,000, 2,500, 5,000, 7,500, 10,000, 25,000, 50,000, 75,000, 100,000, 250,000, 500,000, 750,000, 1,000,000 copies per 1, 10, 100, 1,000 micro liters of a sample or a reaction volume.

As used herein, the terms “first” and “second” are used to distinguish the components of a first reaction (e.g., a “first” reaction; a “first” allele-specific primer) and a second reaction (e.g., a “second” reaction; a “second” allele-specific primer). By convention, as used herein the first reaction amplifies a first (for example, a rare) allelic variant and the second reaction amplifies a second (for example, an abundant) allelic variant or vice versa.

As used herein, both “first allelic variant” and “second allelic variant” can pertain to alleles of a given locus from the same organism. For example, as might be the case in human samples (e.g., cells) comprising wild type alleles, some of which have been mutated to form a minor or rare allele. The first and second allelic variants of the present teachings can also refer to alleles from different organisms. For example, the first allele can be an allele of a genetically modified organism, and the second allele can be the corresponding allele of a wild type organism. The first allelic variants and second allelic variants of the present teachings can be contained on gDNA, as well as mRNA and cDNA, and generally any target nucleic acids that exhibit sequence variability due to, for example, SNP or nucleotide(s) insertion and/or deletion mutations.

As used herein, the term “thermostable” or “thermostable polymerase” refers to an enzyme that is heat stable or heat resistant and catalyzes polymerization of deoxyribonucleotides to form primer extension products that are complementary to a nucleic acid strand. Thermostable DNA polymerases useful herein are not irreversibly inactivated when subjected to elevated temperatures for the time necessary to effect destabilization of single-stranded nucleic acids or denaturation of double-stranded nucleic acids during PCR amplification. Irreversible denaturation of the enzyme refers to substantial loss of enzyme activity. Preferably a thermostable DNA polymerase will not irreversibly denature at about 90°-100° C. under conditions such as is typically required for PCR amplification.

As used herein, the term “PCR amplifying” or “PCR amplification” refers generally to cycling polymerase-mediated exponential amplification of nucleic acids employing primers that hybridize to complementary strands, as described for example in Innis et al., PCR Protocols: A Guide to Methods and Applications, Academic Press (1990). Devices have been developed that can perform thermal cycling reactions with compositions containing fluorescent indicators which are able to emit a light beam of a specified wavelength, read the intensity of the fluorescent dye, and display the intensity of fluorescence after each cycle. Devices comprising a thermal cycler, light beam emitter, and a fluorescent signal detector, have been described, e.g., in U.S. Pat. Nos. 5,928,907; 6,015,674; 6,174,670; and 6,814,934 and include, but are not limited to, the ABI Prism® 7700 Sequence Detection System (Applied Biosystems, Foster City, California), the ABI GeneAmp® 5700 Sequence Detection System (Applied Biosystems, Foster City, California), the ABI GeneAmp® 7300 Sequence Detection System (Applied Biosystems, Foster City, California), the ABI GeneAmp® 7500 Sequence Detection System (Applied Biosystems, Foster City, California), the StepOne™ Real-Time PCR System (Applied Biosystems, Foster City, California) and the ABI GeneAmp® 7900 Sequence Detection System (Applied Biosystems, Foster City, California).

As used herein, the term “Tm”' or “melting temperature” of an oligonucleotide refers to the temperature (in degrees Celsius) at which 50% of the molecules in a population of a single-stranded oligonucleotide are hybridized to their complementary sequence and 50% of the molecules in the population are not-hybridized to said complementary sequence. The Tm of a primer or probe can be determined empirically by means of a melting curve. In some cases it can also be calculated using formulas well know in the art (See, e.g., Maniatis, T., et al., Molecular cloning: a laboratory manual / Cold Spring Harbor Laboratory, Cold Spring Harbor, N.Y.: 1982).

As used herein, the term “sensitivity” refers to the minimum amount (number of copies or mass) of a template that can be detected by a given assay. As used herein, the term “specificity” refers to the ability of an assay to distinguish between amplification from a matched template versus a mismatched template. Frequently, specificity is expressed as ΔCₜ=Ct_{mismatch}−Ct_{match}. An improvement in specificity or “specificity improvement” or “fold difference” is expressed herein as 2^{(ΔCt_condition1-(ΔCt_condition2)}. The term “selectivity” refers to the extent to which an AS-PCR assay can be used to determine minor (often mutant) alleles in mixtures without interferences from major (often wild type) alleles. Selectivity is often expressed as a ratio or percentage. For example, an assay that can detect 1 mutant template in the presence of 100 wild type templates is said to have a selectivity of 1:100 or 1%. As used herein, assay selectivity can also be calculated as ½^{ΔCt} or as a percentage using (½^{ΔCt}×100).

As used herein, the term “Ct” or “Ct value” refers to threshold cycle and signifies the cycle of a PCR amplification assay in which signal from a reporter that is indicative of amplicon generation (e.g., fluorescence) first becomes detectable above a background level. In some embodiments, the threshold cycle or “Ct” is the cycle number at which PCR amplification becomes exponential.

As used herein, the term “delta Ct” or “ΔCt” refers to the difference in the numerical cycle number at which the signal passes the fixed threshold between two different samples or reactions. In some embodiments delta Ct is the difference in numerical cycle number at which exponential amplification is reached between two different samples or reactions. The delta Ct can be used to identify the specificity between a matched primer to the corresponding target nucleic acid sequence and a mismatched primer to the same corresponding target nucleic acid sequence.

In some embodiments, the calculation of the delta Ct value between a mismatched primer and a matched primer is used as one measure of the discriminating power of allele-specific PCR. In general, any factor which increases the difference between the Ct value for an amplification reaction using a primer that is matched to a target sequence (e.g., a sequence comprising an allelic variant of interest) and that of a mismatched primer will result in greater allele discrimination power.

According to various embodiments, a Ct value may be determined using a derivative of a PCR curve. For example, a first, second, or nth order derivative method may be performed on a PCR curve in order to determine a Ct value. In various embodiments, a characteristic of a derivative may be used in the determination of a Ct value. Such characteristics may include, but are not limited by, a positive inflection of a second derivative, a negative inflection of a second derivative, a zero crossing of the second derivative, or a positive inflection of a first derivative. In various embodiments, a Ct value may be determined using a thresholding and baselining method. For example, an upper bound to an exponential phase of a PCR curve may be established using a derivative method, while a baseline for a PCR curve may be determined to establish a lower bound to an exponential phase of a PCR curve. From the upper and lower bound of a PCR curve, a threshold value may be established from which a Ct value is determined. Other methods for the determination of a Ct value known in the art, for example, but not limited by, various embodiments of a fit point method, and various embodiments of a sigmoidal method (See, e.g., U.S. Pat. Nos. 6,303,305; 6,503,720; 6,783,934, 7,228,237 and U.S. Application No. 2004/0096819; the disclosures of which are herein incorporated by reference in their entireties).

### III. Compositions, Methods and Kits

In one aspect, the present invention provides compositions for use in identifying and/or quantitating an allelic variant in a nucleic acid sample. Some of these compositions can comprise: (a) an allele-specific primer; (b) an allele-specific blocker probe; (c) a detector probe; and (d) a locus-specific primer, or any combinations thereof. In some embodiments of the compositions, the compositions may further comprise a polymerase, dNTPs, reagents and/or buffers suitable for PCR amplification, and/or a template sequence or nucleic acid sample. In some embodiments, the polymerase can be thermostable.

In another aspect, the invention provides compositions comprising: (i) a first allele-specific primer, wherein an allele-specific nucleotide portion of the first allele-specific primer is complementary to the first allelic variant of a target sequence; and (ii) a first allele-specific blocker probe that is complementary to a region of the target sequence comprising the second allelic variant, wherein said region encompasses a position corresponding to the binding position of the allele-specific nucleotide portion of the first allele-specific primer, and wherein the first allele-specific blocker probe comprises a minor groove binder.

In some illustrative embodiments, the compositions can further include a locus-specific primer that is complementary to a region of the target sequence that is 3′ from the first allelic variant and on the opposite strand.

In further embodiments, the compositions can further include a detector probe.

In another aspect, the present invention provides methods for amplifying an allele-specific sequence. Some of these methods can include: (a) hybridizing an allele-specific primer to a first nucleic acid molecule comprising a target allele; (b) hybridizing an allele-specific blocker probe to a second nucleic acid molecule comprising an alternative allele wherein the alternative allele corresponds to the same loci as the target allele; (c) hybridizing a locus-specific detector probe to the first nucleic acid molecule; (d) hybridizing a locus-specific primer to the extension product of the allele-specific primer and (e) PCR amplifying the target allele.

In another aspect, the present invention provides methods for detecting and/or quantitating an allelic variant in a mixed sample. Some of these methods can involve: (a) in a first reaction mixture hybridizing a first allele-specific primer to a fist nucleic acid molecule comprising a first allele (allele-1) and in a second reaction mixture hybridizing a second allele-specific primer to a first nucleic acid molecule comprising a second allele (allele-2), wherein the allele-2 corresponds to the same loci as allele-1; (b) in the first reaction mixture hybridizing a first allele-specific blocker probe to a second nucleic acid molecule comprising allele-2 and in the second reaction mixture hybridizing a second allele-specific blocker probe to a second nucleic acid molecule comprising allele-1; (c) in the first reaction mixture, hybridizing a first detector probe to the first nucleic acid molecule and in the second reaction mixture, hybridizing a second detector probe to the first nucleic acid molecule; (d) in the first reaction mixture hybridizing a first locus-specific primer to the extension product of the first allele-specific primer and in the second reaction mixture hybridizing a second locus-specific primer to the extension product of the second allele-specific primer; and (e) PCR amplifying the first nucleic acid molecule to form a first set or sample of amplicons and PCR amplifying the second nucleic acid molecule to form a second set or sample of amplicons; and (f) comparing the first set of amplicons to the second set of amplicons to quantitate allele-1 in the sample comprising allele-2 and/or allele-2 in the sample comprising allele-1.

In yet another aspect, the present invention provides methods for detecting and/or quantitating allelic variants. Some of these methods can comprise: (a) PCR amplifying a first allelic variant in a first reaction comprising (i) a low-concentration first allele-specific primer, (ii) a first locus-specific primer, and (iii) a first blocker probe to form first amplicons; (b) PCR amplifying a second allelic variant in a second reaction comprising (i) a low-concentration second allele-specific primer, (ii) a second locus-specific primer, and (iii) a second blocker probe to form second amplicons; and (d) comparing the first amplicons to the second amplicons to quantitate the first allelic variant in the sample comprising second allelic variants.

In yet another aspect, the present invention provides methods for detecting a first allelic variant of a target sequence in a nucleic acid sample suspected of comprising at least a second allelic variant of the target sequence. Methods of this aspect include forming a first reaction mixture by combining the following: (i) a nucleic acid sample; (ii) a first allele-specific primer, wherein an allele-specific nucleotide portion of the first allele-specific primer is complementary to the first allelic variant of the target sequence; (iii) a first allele-specific blocker probe that is complementary to a region of the target sequence comprising the second allelic variant, wherein said region encompasses a position corresponding to the binding position of the allele-specific nucleotide portion of the first allele-specific primer, and wherein the first allele-specific blocker probe comprises a minor groove binder; (iv) a first locus-specific primer that is complementary to a region of the target sequence that is 3′ from the first allelic variant and on the opposite strand; and (v) a first detector probe.

Next an amplification reaction, typically a PCR amplification reaction, is carried out on the first reaction mixture using the first locus-specific primer and the first allele-specific primer to form a first amplicon. Then, the first amplicon is detected by a change in a detectable property of the first detector probe upon binding to the amplicon, thereby detecting the first allelic variant of the target gene in the nucleic acid sample. The detector probe in some illustrative embodiments is a 5′ nuclease probe. The detectable property in certain illustrative embodiments is fluorescence.

In some embodiments, the 3′ nucleotide position of the 5′ target region of the first allele-specific primer is an allele-specific nucleotide position. In certain other illustrative embodiments, including those embodiments where the 3′ nucleotide position of the 5′ target region of the first allele-specific primer is an allele-specific nucleotide position, the blocking region of the allele-specific primer encompasses the allele-specific nucleotide position. Furthermore, in illustrative embodiments, the first allele-specific blocker probe includes a minor groove binder. Furthermore, the allele-specific blocker probe in certain illustrative embodiments does not have a label, for example a fluorescent label, or a quencher.

In certain illustrative embodiments, the quantity of the first allelic variant is determined by evaluating the change in a detectable property of the first detector probe.

In certain illustrative embodiments, the method further includes forming a second reaction mixture by combining (i) the nucleic acid sample; (ii) a second allele-specific primer, wherein an allele-specific nucleotide portion of the second allele-specific primer is complementary to the second allelic variant of the target sequence; (iii) a second allele-specific blocker probe that is complementary to a region of the target sequence comprising the first allelic variant, wherein said region encompasses a position corresponding to the binding position of the allele-specific nucleotide portion of the second allele-specific primer, and wherein the second allele-specific blocker probe comprises a minor groove binder; (iv) a second locus-specific primer that is complementary to a region of the target sequence that is 3′ from the second allelic variant and on the opposite strand; and (v) a second detector probe. Next, an amplification reaction is carried out on the second reaction mixture using the second allele-specific primer and the locus-specific primer, to form a second amplicon. Then the second amplicon is detected by a change in a detectable property of the detector probe.

In certain embodiments, the method further includes comparing the change in a detectable property of the first detector probe in the first reaction mixture to the change in a detectable property of the second detector probe in the second reaction mixture.

In preferred embodiments, the methods further include a 2-stage cycling protocol. In some embodiments, the cycling protocol comprises a first stage of amplification that employs an initial number of cycles at a lower annealing/extension temperature, followed by a second stage of amplification that employs a number of cycles at a higher annealing/extension temperature. Due to the lower Tm of cast-PCR allele-specific primers (e.g., 53-56° C.), PCR is not optimal at standard annealing/extension conditions (e.g., 60-64° C.). Consequently, lower annealing/extension temperatures are used during the initial cycling stage which improves cast-PCR efficiency significantly.

In some embodiments, the number of cycles used in the first stage of the cast-PCR cycling protocol is fewer than the number of cycles used in the second stage. In some embodiments of the cast-PCR methods, the number of cycles used in the first stage of the cycling protocol is about 2%-20%, 4%-18%, 6%-16%, 8%-14%, 10%-12%, or any percent in between, of the total number of cycles used in the second stage. In some embodiments, the first stage employs between 1 to 10 cycles, 2 to 8 cycles, 3 to 7 cycles, or 4 to 6 cycles, and all number of cycles in between, e.g., 2, 3, 4, 5, 6, or 7 cycles.

In some embodiments, the number of cycles used in the second stage of the cast-PCR cycling protocol is greater than the number of cycles used in the second stage. In some embodiments of the cast-PCR methods, the number of cycles used in the second stage of the cycling protocol is 5 times, 6 times, 8 times, 10 times, 12 times, 18 times, 25 times, or 30 times the number of cycles used in the first stage. In some embodiments, the second stage employs between 30 to 50 cycles, 35 to 48 cycles, 40 to 46 cycles, or any number of cycles in between, e.g., 42, 43, 44, 45, or 46 cycles.

In some embodiments, the lower annealing/extension temperature used during the first cycling stage is about 1° C., about 2° C., about 3° C., about 4° C., or about 5° C. lower than the annealing/extension temperature used during the second cycling stage. In some preferred embodiments, the annealing/extension temperature of the first stage is between 50° C. to 60° C., 52° C. to 58° C., or 54° C. to 56° C., e.g., 53° C., 54° C., 55° C. or 55° C. In some preferred embodiments the annealing/extension temperature of the second stage is between 56° C. to 66° C., 58° C. to 64° C., or 60° C. to 62° C., e.g., 58° C., 60° C., 62° C. or 64° C.

There are several major advantages of this 2-stage PCR cycling protocol used in cast-PCR that make it better than conventional AS-PCR methods. First, it improves the detection sensitivity by lowering the Ct value for matched targets or alleles. Next, it improves the specificity of cast-PCR by increasing the ΔCt between Ct values of matched and mismatched sequences. Finally, it can improve the uniformity of cast-PCR by making it equally efficient across various assays.

In yet another aspect, the present invention provides a reaction mixture that includes the following (i) nucleic acid molecule; (ii) an allele-specific primer, wherein an allele-specific nucleotide portion of the allele-specific primer is complementary to a first allelic variant of a target sequence; (iii) an allele-specific blocker probe that is complementary to a region of the target sequence comprising a second allelic variant, wherein said region encompasses a position corresponding to the binding position of the allele-specific nucleotide portion of the allele-specific primer, and wherein the allele-specific blocker probe comprises a minor groove binder; (iv) a locus-specific primer that is complementary to a region of the target sequence that is 3′ from the first allelic variant and on the opposite strand; and (v) a detector probe.

In certain embodiments, the methods of the invention are used to detect a first allelic variant that is present at a frequency less than 1/10, 1/100, 1/1,000, 1/10,000, 1/100,000, 1/1,000,000, 1/10,000,000, 1/100,000,000 or 1/1,000,000,000, and any fractional ranges in between, of a second allelic variant for a given SNP or gene. In other embodiments, the methods are used to detect a first allelic variant that is present in less than 2, 3, 4, 5, 6, 7, 8, 9, 10, 15, 20, 25, 50, 75, 100, 250, 500, 750, 1,000, 2,500, 5,000, 7,500, 10,000, 25,000, 50,000, 75,000, 100,000, 250,000, 500,000, 750,000, 1,000,000 copies per 1, 10, 100, 1,000 micro liters, and any fractional ranges in between, of a sample or a reaction volume.

In some embodiments, the first allelic variant is a mutant. In some embodiments the second allelic variant is wild type. In some embodiments, the present methods can involve detecting one mutant molecule in a background of at least 1,000 to 1,000,000, such as about 1000 to 10,000, about 10,000 to 100,000, or about 100,000 to 1,000,000 wild type molecules, or any fractional ranges in between. In some embodiments, the methods can provide high sensitivity and the efficiency at least comparable to that of TaqMan®-based assays.

In some embodiments, the comparison of the first amplicons and the second amplicons involving the disclosed methods provides improvements in specificity from 1,000× to 1,000,000× fold difference, such as about 1000 to 10,000×, about 10,000 to 100,000×, or about 100,000 to 1,000,000× fold difference, or any fractional ranges in between. In some embodiments, the size of the amplicons range from about 60-120 nucleotides long.

In another aspect, the present invention provides kits for quantitating a first allelic variant in a sample comprising an alternative second allelic variants that include: (a) a first allele-specific primer; (b) a second allele-specific primer; (c), a first locus-specific primer; (d) a second locus-specific primer; (e) a first allele-specific blocker probe; (f) a second allele-specific blocker probe; and (g) a polymerase. In some embodiments of the disclosed kits, the kit further comprises a first locus-specific detector probe and a second locus-specific detector probe.

In another aspect, the present invention provides kits that include two or more containers comprising the following components independently distributed in one of the two or more containers: (i) a first allele-specific primer, wherein an allele-specific nucleotide portion of the first allele-specific primer is complementary to the first allelic variant of a target sequence; and (ii) a first allele-specific blocker probe that is complementary to a region of the target sequence comprising the second allelic variant, wherein said region encompasses a position corresponding to the binding position of the allele-specific nucleotide portion of the first allele-specific primer, and wherein the first allele-specific blocker probe comprises a minor groove binder.

In some illustrative embodiments, the kits can further include a locus-specific primer that is complementary to a region of the target sequence that is 3′ from the first allelic variant and on the opposite strand.

In other embodiments, the kits can further include a detector probe.

In some embodiments, the compositions, methods, and/or kits can be used in detecting circulating cells in diagnosis. In one embodiment, the compositions, methods, and/or kits can be used to detect tumor cells in blood for early cancer diagnosis. In some embodiments, the compositions, methods, and/or kits can be used for cancer or disease-associated genetic variation or somatic mutation detection and validation. In some embodiments, the compositions, methods, and/or kits can be used for genotyping tera-, tri-and di-allelic SNPs. In other embodiments, the compositions, methods, and/or kits can be used for identifying single or multiple nucleotide insertion or deletion mutations. In some embodiments, the compositions, methods, and/or kits can be used for DNA typing from mixed DNA samples for QC and human identification assays, cell line QC for cell contaminations, allelic gene expression analysis, virus typing/rare pathogen detection, mutation detection from pooled samples, detection of circulating tumor cells in blood, and/or prenatal diagnostics.

In some embodiments, the compositions, methods, and/or kits are compatible with various instruments such as, for example, SDS instruments from Applied Biosystems (Foster City, CA).

### Allele-Specific Primers

Allele-specific primers (ASPs) designed with low Tms exhibit increased discrimination of allelic variants. In some embodiments, the allele-specific primers are short oligomers ranging from about 15-30, such as about 16-28, about 17-26, about 18-24, or about 20-22, or any range in between, nucleotides in length. In some embodiments, the Tm of the allele-specific primers range from about 50° C. to 70° C., such as about 52° C. to 68° C., about 54° C. to 66° C., about 56° C. to 64° C., about 58° C. to 62° C., or any temperature in between (e.g., 53° C., 54° C., 55° C., 56° C.). In other embodiments, the Tm of the allele-specific primers is about 3° C. to 6° C. higher than the anneal/extend temperature of the PCR cycling conditions employed during amplification.

Low allele-specific primer concentration can also improve selectivity. Reduction in concentration of allele-specific primers below 900 nM can increase the delta Ct between matched and mismatched sequences. In some embodiments of the disclosed compositions, the concentration of allele-specific primers ranges from about 20 nM to 900 nM, such as about 50 nM to 700 nM, about 100 nM to 500 nM, about 200 nM to 300 nM, about 400 nM to 500 nM, or any range in between. In some exemplary embodiments, the concentration of the allele-specific primers is between about 200 nM to 400 nM.

In some embodiments, allele-specific primers can comprise an allele-specific nucleotide portion that is specific to the target allele of interest. The allele-specific nucleotide portion of an allele-specific primer is complementary to one allele of a gene, but not another allele of the gene. In other words, the allele-specific nucleotide portion binds to one or more variable nucleotide positions of a gene that is nucleotide positions that are known to include different nucleotides for different allelic variants of a gene. The allele-specific nucleotide portion is at least one nucleotide in length. In exemplary embodiments, the allele-specific nucleotide portion is one nucleotide in length. In some embodiments, the allele-specific nucleotide portion of an allele-specific primer is located at the 3′ terminus of the allele-specific primer. In other embodiments, the allele-specific nucleotide portion is located about 1-2, 3-4, 5-6, 7-8, 9-11, 12-15, or 16-20 nucleotides in from the 3′most-end of the allele-specific primer.

Allele-specific primers designed to target discriminating bases can also improve discrimination of allelic variants. In some embodiments, the nucleotide of the allele-specific nucleotide portion targets a highly discriminating base (e.g., for detection of A/A, A/G, G/A, G/G, A/C, or C/A alleles). Less discriminating bases, for example, may involve detection of C/C, T/C, G/T, T/G, C/T alleles. In some embodiments, for example when the allele to be detected involves A/G or C/T SNPs, A or G may be used as the 3′ allele-specific nucleotide portion of the allele-specific primer (e.g., if A or T is the major allele), or C or T may be used as the 3′ allele-specific nucleotide portion of the allele-specific primer (e.g., if C or G is the major allele). In other embodiments, A may be used as the nucleotide-specific portion at the 3′ end of the allele specific primer (e.g., the allele-specific nucleotide portion) when detecting and/or quantifying A/T SNPs. In other embodiments, G may be used as the nucleotide-specific portion at the 3′ end of the allele specific primer when detecting and/or quantifying C/G SNPs.

In some embodiments, the allele-specific primer can comprise a target-specific portion that is specific to the polynucleotide sequence (or locus) of interest. In some embodiments the target-specific portion is about 75-85%, 85-95%, 95-99% or 100% complementary to the target polynucleotide sequence of interest. In some embodiments, the target-specific portion of the allele-specific primer can comprise the allele-specific nucleotide portion. In other embodiments, the target-specific portion is located 5′ to the allele-specific nucleotide portion. The target-specific portion can be about 4-30, about 5-25, about 6-20, about 7-15, or about 8-10 nucleotides in length. In some embodiments, the Tm of the target specific portion is about 5° C. below the anneal/extend temperature used for PCR cycling. In some embodiments, the Tm of the target specific portion of the allele-specific primer ranges from about 51° C. to 60° C., about 52° C. to 59° C., about 53° C. to 58° C., about 54° C. to 57° C., about 55° C. to 56° C., or about 50° C. to about 60° C.

In some embodiments of the disclosed methods and kits, the target-specific portion of the first allele-specific primer and the target-specific portion of the second allele-specific primer comprise the same sequence. In other embodiments, the target-specific portion of the first allele-specific primer and the target-specific portion of the second allele-specific primer are the same sequence.

In some embodiments, the allele-specific primer comprises a tail. Allele-specific primers comprising tails, enable the overall length of the primer to be reduced, thereby lowering the Tm without significant impact on assay sensitivity.

In some exemplary embodiments, the tail is on the 5′ terminus of the allele-specific primer. In some embodiments, the tail is located 5′ of the target-specific portion and/or allele-specific nucleotide portion of the allele-specific primer. In some embodiments, the tail is about 65-75%, about 75-85%, about 85-95%, about 95-99% or about 100% non-complementary to the target polynucleotide sequence of interest. In some embodiments the tail can be about 2-40, such as about 4-30, about 5-25, about 6-20, about 7-15, or about 8-10 nucleotides in length. In some embodiments the tail is GC-rich. For example, in some embodiments the tail sequence is comprised of about 50-100%, about 60-100%, about 70-100%, about 80-100%, about 90-100% or about 95-100% G and/or C nucleotides.

The tail of the allele-specific primer may be configured in a number of different ways, including, but not limited to a configuration whereby the tail region is available after primer extension to hybridize to a complementary sequence (if present) in a primer extension product. Thus, for example, the tail of the allele-specific primer can hybridize to the complementary sequence in an extension product resulting from extension of a locus-specific primer.

In some embodiments of the disclosed methods and kits, the tail of the first allele-specific primer and the tail of the second allele-specific primer comprise the same sequence. In other embodiments, the 5′ tail of the first allele-specific primer and the 5′ tail of the second allele-specific primer are the same sequence.

### Allele-Specific Blocker Probes

Allele-specific blocker probes (or ASBs) (herein sometimes referred to as “blocker probes”) may be designed as short oligomers that are single-stranded and have a length of 100 nucleotides or less, more preferably 50 nucleotides or less, still more preferably 30 nucleotides or less and most preferably 20 nucleotides or less with a lower limit being approximately 5 nucleotides.

In some embodiments, the Tm of the blocker probes range from 58° C. to 70° C., 61° C. to 69° C., 62° C. to 68° C., 63° C. to 67° C., 64° C. to 66° C., or about 60° C. to about 63° C., or any range in between. In yet other embodiments, the Tm of the allele-specific blocker probes is about 3° C. to 6° C. higher than the anneal/extend temperature in the PCR cycling conditions employed during amplification.

In some embodiments, the blocker probes are not cleaved during PCR amplification. In some embodiments, the blocker probes comprise a non-extendable blocker moiety at their 3′-ends. In some embodiments, the blocker probes can further comprise other moieties (including, but not limited to additional non-extendable blocker moieties, quencher moieties, fluorescent moieties, etc) at their 3′-end, 5′-end, and/or any internal position in between. In some embodiments, the allele position is located about 5-15, such as about 5-11, about 6-10, about 7-9, about 7-12, or about 9-11, such as about 6, about 7, about 8, about 9, about 10, or about 11 nucleotides away from the non-extendable blocker moiety of the allele-specific blocker probes when hybridized to their target sequences. In some embodiments, the non-extendable blocker moiety can be, but is not limited to, an amine (NH₂), biotin, PEG, DPI₃, or PO₄. In some preferred embodiments, the blocker moiety is a minor groove binder (MGB) moiety. (The oligonucleotide-MGB conjugates of the present invention are hereinafter sometimes referred to as “MGB blocker probes” or “MGB blockers.”)

As disclosed herein, the use of MGB moieties in allele-specific blocker probes can increase the specificity of allele-specific PCR. One possibility for this effect is that, due to their strong affinity to hybridize and strongly bind to complementary sequences of single or double stranded nucleic acids, MGBs can lower the Tm of linked oligonucleotides (See, for example, Kutyavin, I., et al., Nucleic Acids Res., 2000, Vol. 28, No. 2: 655-661). Oligonucleotides comprising MGB moieties have strict geometric requirements since the linker between the oligonucleotide and the MGB moiety must be flexible enough to allow positioning of the MGB in the minor groove after DNA duplex formation. Thus, MGB blocker probes can provide larger Tm differences between matched versus mismatched alleles as compared to conventional DNA blocker probes.

In general, MGB moieties are molecules that bind within the minor groove of double stranded DNA. Although a generic chemical formula for all known MGB compounds cannot be provided because such compounds have widely varying chemical structures, compounds which are capable of binding in the minor groove of DNA, generally speaking, have a crescent shape three dimensional structure. Most MGB moieties have a strong preference for A-T (adenine and thymine) rich regions of the B form of double stranded DNA. Nevertheless, MGB compounds which would show preference to C-G (cytosine and guanine) rich regions are also theoretically possible. Therefore, oligonucleotides comprising a radical or moiety derived from minor groove binder molecules having preference for C-G regions are also within the scope of the present invention.

Some MGBs are capable of binding within the minor groove of double stranded DNA with an association constant of 10³ M⁻¹ or greater. This type of binding can be detected by well established spectrophotometric methods such as ultraviolet (UV) and nuclear magnetic resonance (NMR) spectroscopy and also by gel electrophoresis. Shifts in UV spectra upon binding of a minor groove binder molecule and NMR spectroscopy utilizing the “Nuclear Overhauser” (NOSEY) effect are particularly well known and useful techniques for this purpose. Gel electrophoresis detects binding of an MGB to double stranded DNA or fragment thereof, because upon such binding the mobility of the double stranded DNA changes.

A variety of suitable minor groove binders have been described in the literature. See, for example, Kutyavin, et al. U.S. Pat. No. 5,801,155; Wemmer, D. E., and Dervan P. B., Current Opinion in Structural Biology, 7:355-361 (1997); Walker, W. L., Kopka, J. L. and Goodsell, D. S., Biopolymers, 44:323-334 (1997); Zimmer, C.& Wahnert, U. Prog. Biophys. Molec. Bio. 47:31-112 (1986) and Reddy, B. S. P., Dondhi, S. M., and Lown, J. W., Pharmacol. Therap., 84:1-111 (1999). In one group of embodiments, the MGB is selected from the group consisting of CC1065 analogs, lexitropsins, distamycin, netropsin, berenil, duocarmycin, pentamidine, 4, 6-diamino-2-phenylindole and pyrrolo[2,1-c][1,4]benzodiazepines. A preferred MGB in accordance with the present disclosure is DPI₃ (see U.S. Pat. No. 6,727,356, the disclosure of which is incorporated herein by reference in its entirety).

Suitable methods for attaching MGBs through linkers to oligonucleotides or probes and have been described in, for example, U.S. Pat. Nos. 5,512,677; 5,419,966; 5,696,251; 5,585,481; 5,942,610; 5,736,626; 5,801,155 and 6,727,356. (The disclosures of each of which are incorporated herein by reference in their entireties.) For example, MGB-oligonucleotide conjugates can be synthesized using automated oligonucleotide synthesis methods from solid supports having cleavable linkers. In other examples, MGB probes can be prepared from an MGB modified solid support substantially in accordance with the procedure of Lukhtanov et al. Bioconjugate Chem., 7: 564-567 (1996). (The disclosure of which is also incorporated herein by reference in its entirety.) According to these methods, one or more MGB moieties can be attached at the 5′-end, the 3′-end and/or at any internal portion of the oligonucleotide.

The location of an MGB moiety within an MGB-oligonucleotide conjugate can affect the discriminatory properties of such a conjugate. An unpaired region within a duplex will likely result in changes in the shape of the minor groove in the vicinity of the mismatched base(s). Since MGBs fit best within the minor groove of a perfectly-matched DNA duplex, mismatches resulting in shape changes in the minor groove would reduce binding strength of an MGB to a region containing a mismatch. Hence, the ability of an MGB to stabilize such a hybrid would be decreased, thereby increasing the ability of an MGB-oligonucleotide conjugate to discriminate a mismatch from a perfectly-matched duplex. On the other hand, if a mismatch lies outside of the region complementary to an MGB-oligonucleotide conjugate, discriminatory ability for unconjugated and MGB-conjugated oligonucleotides of equal length is expected to be approximately the same. Since the ability of an oligonucleotide probe to discriminate single base pair mismatches depends on its length, shorter oligonucleotides are more effective in discriminating mismatches. The first advantage of the use of MGB-oligonucleotides conjugates in this context lies in the fact that much shorter oligonucleotides compared to those used in the art (i.e., 20-mers or shorter), having greater discriminatory powers, can be used, due to the pronounced stabilizing effect of MGB conjugation. Consequently, larger delta Tms of allele-specific blocker probes can improve AS-PCR assay specificity and selectivity.

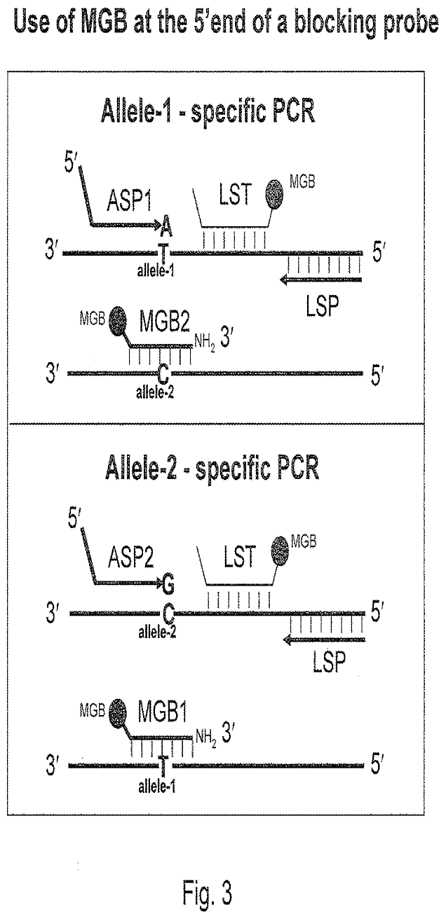

Blocker probes having MGB at the 5′ termini may have additional advantages over other blocker probes having a blocker moiety (e.g., MGB, PO₄, NH₂, PEG, or biotin) only at the 3′ terminus. This is at least because blocker probes having MGB at the 5′ terminus (in addition to a blocking moiety at the 3′-end that prevents extension) will not be cleaved during PCR amplification. Thus, the probe concentration can be maintained at a constant level throughout PCR, which may help maintain the effectiveness of blocking non-specific priming, thereby increasing cast-PCR assay specificity and selectivity (FIG. 3).

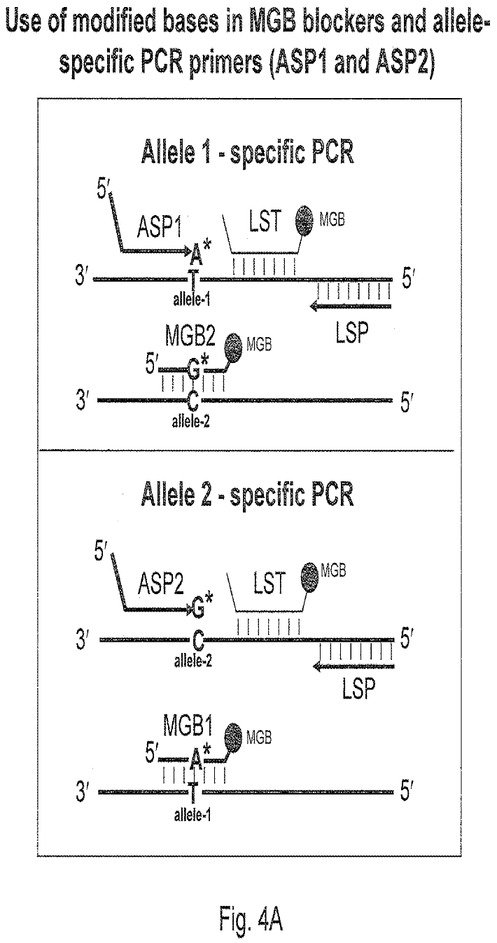

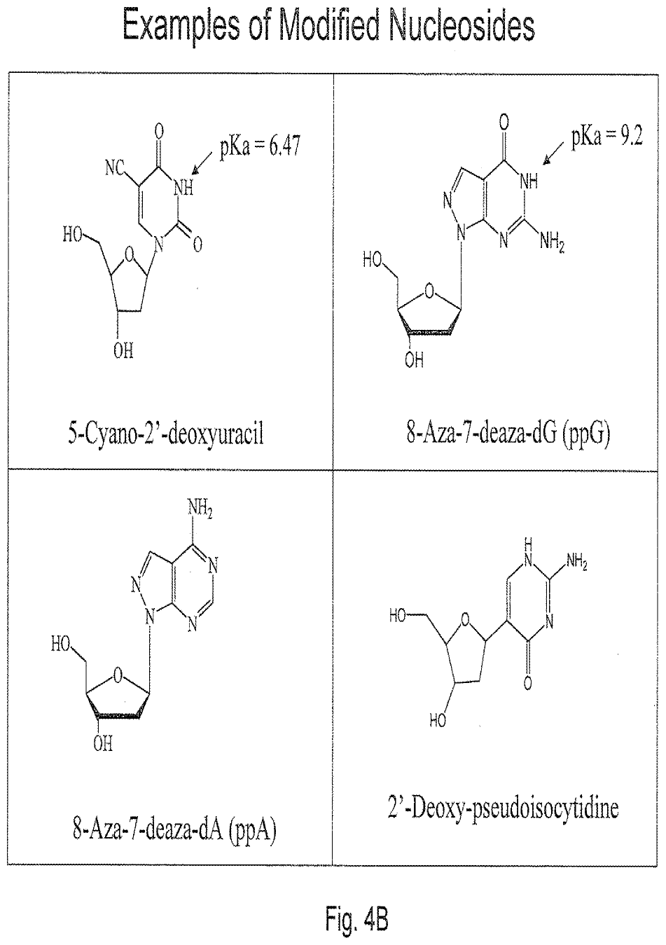

In some embodiments, as depicted in FIG. 4A, the allele-specific primer and/or the allele-specific blocker probe can comprise one or more modified nucleobases or nucleosidic bases different from the naturally occurring bases (i.e., adenine, cytosine, guanine, thymine and uracil). In some embodiments, the modified bases are still able to effectively hybridize to nucleic acid units that contain adenine, guanine, cytosine, uracil or thymine moieties. In some embodiments, the modified base(s) may increase the difference in the Tm between matched and mismatched target sequences and/or decrease mismatch priming efficiency, thereby improving not only assay specificity, bust also selectivity.

Modified bases are considered to be those that differ from the naturally-occurring bases by addition or deletion of one or more functional groups, differences in the heterocyclic ring structure (i.e., substitution of carbon for a heteroatom, or vice versa), and/or attachment of one or more linker arm structures to the base. In some embodiments, all tautomeric forms of naturally occurring bases, modified bases and base analogues may also be included in the oligonucleotide primers and probes of the invention.

Some examples of modified base(s) may include, for example, the general class of base analogues 7-deazapurines and their derivatives and pyrazolopyrimidines and their derivatives (described in PCT WO 90/14353; and U.S. application Ser. No. 09/054,630, the disclosures of each of which are incorporated herein by reference in their entireties). Examples of base analogues of this type include, for example, the guanine analogue 6-amino-1H-pyrazolo[3,4-d]pyrimidin-4(5H)-one (ppG), the adenine analogue 4-amino-1H-pyrazolo[3,4-d]pyrimidine (ppA), and the xanthine analogue 1H-pyrazolo[4,4-d]pyrimidin-4(5H)-6(7H)-dione (ppX). These base analogues, when present in an oligonucleotide of some embodiments of this invention, strengthen hybridization and can improve mismatch discrimination.

Additionally, in some embodiments, modified sugars or sugar analogues can be present in one or more of the nucleotide subunits of an oligonucleotide conjugate in accordance with the invention. Sugar modifications include, but are not limited to, attachment of substituents to the 2′,3′ and/or 4′ carbon atom of the sugar, different epimeric forms of the sugar, differences in the α- or β-configuration of the glycosidic bond, and other anomeric changes. Sugar moieties include, but are not limited to, pentose, deoxypentose, hexose, deoxyhexose, ribose, deoxyribose, glucose, arabinose, pentofuranose, xylose, lyxose, and cyclopentyl.

Locked nucleic acid (LNA)-type modifications, for example, typically involve alterations to the pentose sugar of ribo- and deoxyribonucleotides that constrains, or “locks,” the sugar in the N-type conformation seen in A-form DNA. In some embodiments, this lock can be achieved via a 2′-O, 4′-C methylene linkage in 1,2:5,6-di-O-isopropylene-α-D-allofuranose. In other embodiments, this alteration then serves as the foundation for synthesizing locked nucleotide phosphoramidite monomers. (See, for example, Wengel J., Acc. Chem. Res., 32:301-310 (1998), U.S. Pat. No. 7,060,809; Obika, et al., Tetrahedron Lett 39: 5401-5405 (1998); Singh, et al., Chem Commun 4:455-456 (1998); Koshkin, et al., Tetrahedron 54: 3607-3630 (1998), the disclosures of each of which are incorporated herein by reference in their entireties.)

In some preferred embodiments, the modified bases include 8-Aza-7-deaza-dA (ppA), 8-Aza-7-deaza-dG (ppG), 2′-Deoxypseudoisocytidine (iso dC), 5-fluoro-2′-deoxyuridine (fdU), locked nucleic acid (LNA), or 2′-O, 4′-C-ethylene bridged nucleic acid (ENA) bases. Other examples of modified bases that can be used in the invention are depicted in FIG. 4B and described in U.S. Pat. No. 7,517,978 (the disclosure of which is incorporated herein by reference in its entirety).

Many modified bases, including for example, LNA, ppA, ppG, 5-Fluoro-dU (fdU), are commercially available and can be used in oligonucleotide synthesis methods well known in the art. In some embodiments, synthesis of modified primers and probes can be carried out using standard chemical means also well known in the art. For example, in certain embodiments, the modified moiety or base can be introduced by use of a (a) modified nucleoside as a DNA synthesis support, (b) modified nucleoside as a phosphoramidite, (c) reagent during DNA synthesis (e.g., benzylamine treatment of a convertible amidite when incorporated into a DNA sequence), or (d) by post-synthetic modification.

In some embodiments, the primers or probes are synthesized so that the modified bases are positioned at the 3′ end. In some embodiments, the modified base are located between, 1-6 nucleotides, e.g., 2, 3, 4 or 5 nucleotides away from the 3′-end of the allele-specific primer or blocker probe. In some preferred embodiments, the primers or probes are synthesized so that the modified bases are positioned at the 3′-most end of the allele-specific primer or blocker probe.

Modified internucleotide linkages can also be present in oligonucleotide conjugates of the invention. Such modified linkages include, but are not limited to, peptide, phosphate, phosphodiester, phosphotriester, alkylphosphate, alkanephosphonate, thiophosphate, phosphorothioate, phosphorodithioate, methylphosphonate, phosphoramidate, substituted phosphoramidate and the like. Several further modifications of bases, sugars and/or internucleotide linkages, that are compatible with their use in oligonucleotides serving as probes and/or primers, will be apparent to those of skill in the art.

In addition, in some embodiments, the nucleotide units which are incorporated into the oligonucleotides of the allele-specific primers and/or blocker probes of the present invention may have a cross-linking function (an alkylating agent) covalently bound to one or more of the bases, through a linking arm.

In some embodiments of the methods and kits, the first allele-specific blocker probe binds to the same strand or sequence as the first allele-specific primer, while the second allele-specific blocker probe binds to the opposite strand and/or complementary sequence as the first allele-specific primer.

### Detector Probes

In some embodiments, detector probe is designed as short oligomers ranging from about 15-30 nucleotides, such as about 16, about 18, about 22, about 24, about 30, or any number in between. In some embodiments, the Tm of the detector probe ranges from about 60° C. to 80° C., about 61° C. to 69° C., about 62° C. to 68° C., about 63° C. to 67° C., or about 64° C. to 66° C., or any range in between.

In some embodiments, the detector probe is a locus-specific detector probes (LST). In other embodiments the detector probe is a 5′ nuclease probe. In some exemplary embodiments, the detector probe can comprises an MGB moiety, a reporter moiety (e.g., FAM™, TET™, JOE™ VIC™, or SYBR® Green), a quencher moiety (e.g., Black Hole Quencher™ or TAMRA™;), and/or a passive reference (e.g., ROX™). In some exemplary embodiments, the detector probe is designed according to the methods and principles described in U.S. Pat. No. 6,727,356 (the disclosure of which is incorporated herein by reference in its entirety). In some exemplary embodiments, the detector probe is a TaqMan® probe (Applied Biosystems, Foster City). In exemplary embodiments, the locus-specific detector probe can be designed according to the principles and methods described in U.S. Pat. No. 6,727,356 (the disclosure of which is incorporated herein by reference in its entirety). For example, fluorogenic probes can be prepared with a quencher at the 3′ terminus of a single DNA strand and a fluorophore at the 5′ terminus. In such an example, the 5′-nuclease activity of a Taq DNA polymerase can cleave the DNA strand, thereby separating the fluorophore from the quencher and releasing the fluorescent signal. In some embodiments, the detector probes are hybridized to the template strands during primer extension step of PCR amplification (e.g., at 60-65° C.). In yet other embodiments, an MGB is covalently attached to the quencher moiety of the locus-specific detector probes (e.g., through a linker).

In some embodiments of the disclosed methods and kits, the first and second detector probes are the same and/or comprise the same sequence or are the same sequence.

### Locus-Specific Primers

In some embodiments, locus-specific primer (LSP) is designed as a short oligomer ranging from about 15-30 nucleotides, such as about 16, about 18, about 22, about 24, about 30, or any number in between. In some embodiments, the Tm of the locus-specific primer ranges from about 60° C. to 70° C., about 61° C. to 69° C., about 62° C. to 68° C., about 63° C. to 67° C., or about 64° C. to 66° C., or any range in between.

In some other embodiments of the disclosed methods and kits, the first locus-specific detector probe and/or second locus-specific detector probes comprise the same sequence or are the same sequence.

### Additional Components

Polymerase enzymes suitable for the practice of the present invention are well known in the art and can be derived from a number of sources. Thermostable polymerases may be obtained, for example, from a variety of thermophilic bacteria that are commercially available (for example, from American Type Culture Collection, Rockville, Md.) using methods that are well-known to one of ordinary skill in the art (See, e.g., U.S. Pat. No. 6,245,533). Bacterial cells may be grown according to standard microbiological techniques, using culture media and incubation conditions suitable for growing active cultures of the particular species that are well-known to one of ordinary skill in the art (See, e.g., Brock, T. D., and Freeze, H., J. Bacteriol. 98(1):289-297 (1969); Oshima, T., and Imahori, K, Int. J. Syst. Bacteriol. 24(1):102-112 (1974)). Suitable for use as sources of thermostable polymerases are the thermophilic bacteria Thermus aquaticus, Thermus thermophilus, Thermococcus litoralis, Pyrococcus furiosus, Pyrococcus woosii and other species of the Pyrococcus genus, Bacillus stearothermophilus, Sulfolobus acidocaldarius, Thermoplasma acidophilum, Thermus flavus, Thermus ruber, Thermus brockianus, Thermotoga neapolitana, Thermotoga maritima and other species of the Thermotoga genus, and Methanobacterium thermoautotrophicum, and mutants of each of these species. Preferable thermostable polymerases can include, but are not limited to, Taq DNA polymerase, Tne DNA polymerase, Tma DNA polymerase, or mutants, derivatives or fragments thereof.

### Various Sources and/or Preparation Methods of Nucleic Acids

Sources of nucleic acid samples in the disclosed compositions, methods and/or kits include, but are not limited to, human cells such as circulating blood, buccal epithelial cells, cultured cells and tumor cells. Also other mammalian tissue, blood and cultured cells are suitable sources of template nucleic acids. In addition, viruses, bacteriophage, bacteria, fungi and other micro-organisms can be the source of nucleic acid for analysis. The DNA may be genomic or it may be cloned in plasmids, bacteriophage, bacterial artificial chromosomes (BACs), yeast artificial chromosomes (YACs) or other vectors. RNA may be isolated directly from the relevant cells or it may be produced by in vitro priming from a suitable RNA promoter or by in vitro transcription. The present invention may be used for the detection of variation in genomic DNA whether human, animal or other. It finds particular use in the analysis of inherited or acquired diseases or disorders. A particular use is in the detection of inherited diseases.

In some embodiments, template sequence or nucleic acid sample can be gDNA. In other embodiments, the template sequence or nucleic acid sample can be cDNA. In yet other embodiments, as in the case of simultaneous analysis of gene expression by RT-PCR, the template sequence or nucleic acid sample can be RNA. The DNA or RNA template sequence or nucleic acid sample can be extracted from any type of tissue including, for example, formalin-fixed paraffin-embedded tumor specimens.

### Preamplification

In some embodiments, additional compositions, methods and kits are provided for “boosting” cast-PCR amplification reactions for limited quantity specimens having very low nucleic acid copy number. In some embodiments, said compositions, methods and kits involve a two-step amplification process comprising a first “booster” or pre-amplification multiplex reaction (see, for example, U.S. Pat. Nos. 6,605,451 and 7,087,414 and U.S. Published Application No. 2004/0175733, the disclosures of which are herein incorporated by reference in their entireties), followed by a second single-plex (i.e., cast-PCR) amplification reaction.

In some preferred embodiments, the first step involves a multiplex reaction which uses at least two complete sets of primers (e.g., one forward allele-1-specific primer, one forward allele-2-specific primer and one reverse locus-specific primer), each set of which is suitable or operative for amplifying a specific polynucleotide of interest. In other embodiments, the resultant multiplex products acquired in the first step are divided into optimized secondary single-plex cast-PCR amplification reactions, each containing at least one primer set previously used in the first multiplexing step and then PCR amplified using the cast-PCR methods described herein.

In other preferred embodiments, the first multiplex reaction is a cast-PCR amplification reaction (although other well known amplification methods such as, but not limited to PCR, RT-PCR, NASBA, SDA, TMA, CRCA, Ligase Chain Reaction, etc. can be used). In certain embodiments, the first multiplex reaction comprises a plurality of allele-specific primers, and locus-specific primers, each group of which is specific for a particular allele of interest and designed according to the cast-PCR methods described herein. Unlike single-plex cast-PCR reactions that generate a single amplified sequence, multiplex cast-PCR amplification reactions, by virtue of utilizing a plurality of different primer sets, can permit the simultaneous amplification of a plurality of different sequences of interest in a single reaction. Because a plurality of different sequences is amplified simultaneously in a single reaction, the multiplex amplifications can effectively increase the concentration or quantity of a sample available for downstream cast-PCR assays. Thus, in some preferred embodiments, significantly more analyses or assays can be performed with a pre-amplified cast-PCR sample than could have been performed with the original sample.

The number of different amplification primer pairs utilized in the multiplex amplification is not critical and can range from as few as two, to as many as tens, hundreds, thousands, or even more. Thus, depending upon the particular conditions, the multiplex amplifications permit the simultaneous amplification of from as few as two to as many as tens, hundreds, thousands, or even more polynucleotide sequences of interest.

The number of amplification cycles performed with a multiplex amplification may depend upon, among other factors, the degree of amplification desired. The degree of amplification desired, in turn, may depend upon such factors as the amount of polynucleotide sample to be amplified or the number of alleles or mutations to be detected using subsequent cast-PCR assays.

In preferred embodiments, it may be desirable to keep the multiplex amplification from progressing beyond the exponential phase or the linear phase. Indeed, in some embodiments, it may be desirable to carry out the multiplex amplification for a number of cycles suitable to keep the reaction within the exponential or linear phase. Utilization of a truncated multiplex amplification round can result in a sample having a boosted product copy number of about 100-1000 fold increase.

In many embodiments, pre-amplification permits the ability to perform cast-PCR assays or analyses that require more sample, or a higher concentration of sample, than was originally available. For example, after a 10×, 100×, 1000×, 10,000×, and so on, multiplex amplification, subsequent cast-PCR single-plex assays can then be performed using, respectively, a 10×, 100×, 1000×, 10,000×, and so on, less sample volume. In some embodiments, this allows each single-plex cast-PCR reaction to be optimized for maximum sensitivity and requires only one method of detection for each allele analyzed. This can be a significant benefit to cast-PCR analysis since, in some embodiments, it allows for the use of off-the-shelf commercially available cast-PCR reagents and kits to be pooled together and used in a multiplex amplification reaction without extensive effort toward or constraints against redesigning and/or re-optimizing cast-PCR assays for any given target sequence. Moreover, in some embodiments, the ability to carry out a multiplex amplification with reagents and kits already optimized for cast-PCR analysis permits the creation of multiplex amplification reactions that are ideally correlated or matched with subsequent single-plex cast-PCR assays. The following examples are intended to illustrate but not limit the invention.

### EXAMPLES

### I. General Cast-PCR Assay Design

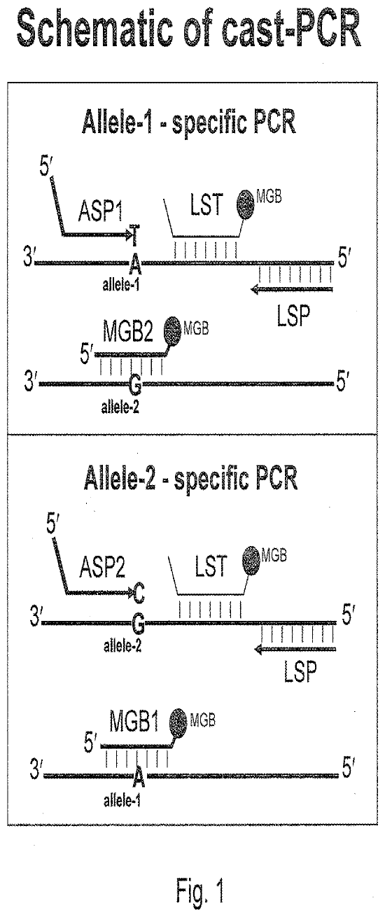

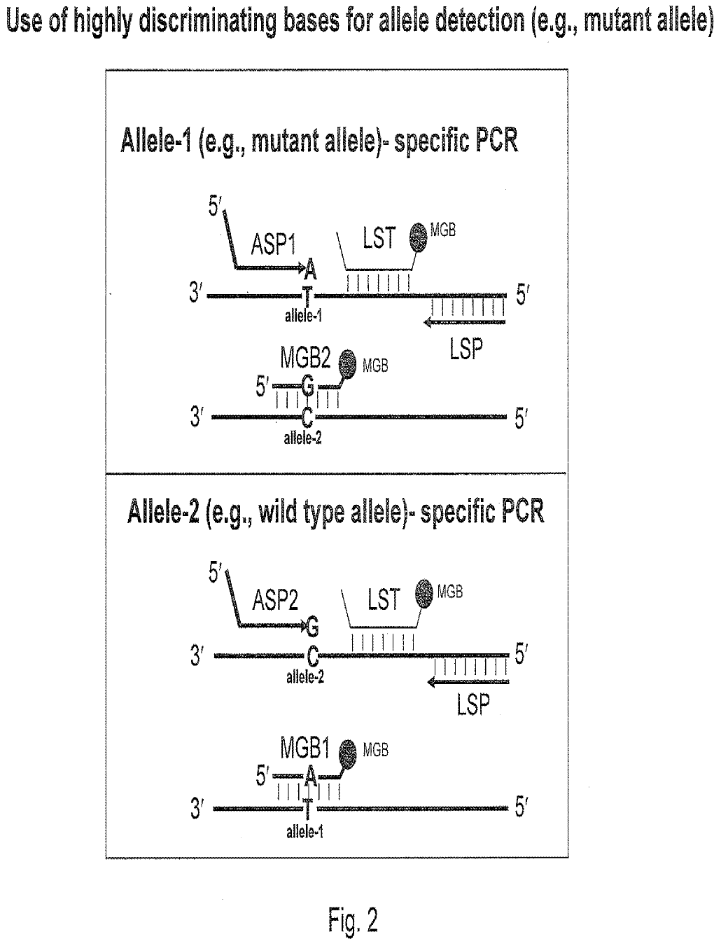

The general schema for the cast-PCR assays used in the following examples is illustrated in FIG. 1. For each SNP that was analyzed, allele-specific primers (ASPs) were designed to target a first allele (i.e. allele-1) and a second allele (i.e. allele-2). The cast-PCR assay reaction mixture for allele-1 analysis included a 5′-tailed allele-1-specific primer (ASP1), one MGB allele-2 blocker probe (MGB2), one common locus-specific TaqMan probe (LST) and one common locus-specific primer (LSP). The cast-PCR assay reaction mixture for analysis of allele-2 included a 5′-tailed allele-2-specific primer (ASP2), one MGB allele-1 blocker probe (MGB1), one common locus-specific TaqMan probe (LST) and one common locus-specific primer (LSP).

### II. Reaction Conditions

Each assay reaction mixture (10 μl total) contained 1× TaqMan Genotyping Master Mixture (Applied Biosystems, Foster City, CA; P/N 437135), 0.5 ng/μL genomic DNA or 1 million copies of plasmid DNA (or as indicated otherwise), 300 nM (unless specified otherwise) tailed-, or in some cases untailed-, allele-specific primer (ASP1 for detection of allele-1 or ASP2 for detection of allele-2), 200 nM TaqMan probe (LST), 900 nM locus-specific primer (LSP), 150 nM allele-specific MGB blocker probe (MGB1 for detection of allele-2 or MGB2 for detection of allele-1). The reactions were incubated in a 384-well plate at 95° C. for 10 minutes, then for 5 cycles at 95° C. for 15 seconds and 58° C. for 1 minute, then by 45 cycles at 95° C. for 15 seconds and 60° C. for 1 minute. All reactions were run in duplicate or higher replication in an ABI PRISM 7900HT® Sequence Detection System, according to the manufacturer's instructions.

The 2-stage cycling protocol used in the following examples for cast-PCR amplification reactions is different from conventional allele-specific PCR (AS-PCR). The 2-stage cycling protocol comprises an initial 5 cycles at a lower annealing/extension temperature (e.g., 58° C.), followed by 45 standard cycles at a higher annealing/extension temperature (e.g., 60° C.). Due to the lower Tm of cast-PCR allele-specific primers (e.g., 53-56° C.), PCR is not optimal at standard annealing/extension conditions (e.g., 60° C.). Consequently, lower annealing/extension temperatures used during the initial 5 cycles increases overall cast-PCR efficiency.

### III. Nucleic Acid Samples

Plasmids containing specific SNP sequences were designed and ordered from BlueHeron (Bothell, WA). (See Table 1 for a list of plasmids comprising SNPs used in some of the following examples.) The plasmids were quantified using TaqMan RNase P Assay (Applied Biosystems, Foster City, CA; P/N 4316838) according to the manufacturer's instructions and were used as templates (See Table 1, RNase P Control) to validate sensitivity, linear dynamic range, specificity, and selectivity of the given assays.

Genomic DNAs were purchased from Coriell Institute for Medical Research (Camden, NJ; NA17203, NA17129, NA17201). The genotypes of target SNPs were validated with TaqMan SNP Genotyping Assays (Applied Biosystems, Foster City, CA; P/N 4332856) according to the manufacturer's instructions.

### IV. Modified Oligonucleotides

Modified bases were purchased from Berry and Associates (ppA: P/N BA 0239; ppG: P/N BA 0242; fdU: P/N BA 0246; and iso dC: P/N BA 0236) or Exicon (LNA-T Amidite: P/N EQ-0064; LNA-mC Amidite: P/N EQ-0066; LNA-G Amidite: P/N EQ-0082; and LNA-A Amidite: P/N EQ-0063). Oligonucleotides comprising the modified nucleotides at their 3′ends were synthesized according to the manufacturer's instructions.

TABLE 1
Plasmid SNP Sequences (target alleles
are indicated in brackets).
Table 1 discloses SEQ ID NOS
1-27, respectively, in order of appearance.

| SNP ID | Sequence |
|---|---|
| CV11201742 | GCTCTGCTTCATTCCTGTCTGAAGAAGGGCA |
| | GATAGTTTGGCTGCTCCTGTG[C/T]TGTCA |
| | CCTGCAATTCTCCCTTATCAGGGCCATTGGC |
| | CTCTCCCTTCTCTCTGTGAGGGATATTTTCT |
| | CTGACTTGTCAATCCACATCTTCC |
| CV11349123 | GGCTTGCAATGGCTCCAACCGGAAGGGCGGT |
| | GCTCGAGCTGTGGTGCGTGC[C/T]GCTAAG |
| | TTGTGCGTTCCAGGGTGCACTCGC |
| CV1207700 | GCAACTATACCCTTGATGGATGGAGATTT |
| | A[C/T]GCAATGTGTTTTACTGGGTAGAGTG |
| | ACAGACCTT |
| CV25594064 | CCTGAACTTATTTGGCAAGAGCGATGAGTAC |
| | TCTTAAAATTACTATCTGGAAATTATATTAT |
| | TTAGAATCTGCCAATTACCTAGATCCCCCC |
| | T[C/G]AACAATTGTTTCACCAAGGAACTT |
| | CCTGAA |
| CV25639181 | GAATTGGTTGTCTCCTTATGGGAACTGGAAG |
| | TATTTTGACA[G/T]CTTTACCACATTTCTT |
| | CATGGGATAGTAAGTGTTAAACAGCTCTGAG |
| | CCATTTATTATCAGCTACTTGTAAATTAGCA |
| | GTAGAATTTTATTTTTATACTTGTAAGTGGG |
| | CAGTTACCTTTTGAGAGGAATACCTATAG |
| RNaseP Control | GCGGAGGGAAGCTCATCAGTGGGGCCACGAG |
| | CTGAGTGCGTCCTGTCACTCCACTCCCATGT |
| | CCCTTGGGAAGGTCTGAGACTAGGG |
| BRAF-1799TA | TACTACACCTCAGATATATTTCTTCATGAAG |
| | ACCTCACAGTAAAAATAGGTGATTTTGGTCT |
| | AGCTACAG[T/A]GAAATCTCGATGGAGTGG |
| | GTCCCATCAGTTTGAACAGTTGTCTGGATCC |
| | ATTTTGTGGATGGTAAGAATTGAGGCTATTT |
| | TTCCACTGATTAAATTTTTGGCCCTGAGATG |
| | CTGCTGAGTT |
| CTNNB1-121AG | TGCTAATACTGTTTCGTATTTATAGCTGATT |
| | TGATGGAGTTGGACATGGCCATGGAACCAGA |
| | CAGAAAAGCGGCTGTTAGTCACTGGCAGCAA |
| | CAGTCTTACCTGGACTCTGGAATCCATTCTG |
| | GTGCCACT[A/G]CCACAGCTCCTTCTCTGA |
| | GTGGTAAAGGCAATCCTGAGGAAGAGGATGT |
| | GGATACCTCCCAAGTC |
| CTNNB1-134CT | TTTGATGGAGTTGGACATGGCCATGGAACCA |
| | GACAGAAAAGCGGCTGTTAGTCACTGGCAGC |
| | AACAGTCTTACCTGGACTCTGGAATCCATTC |
| | TGGTGCCACTACCACAGCTCCTT[C/T]TCT |
| | GAGTGGTAAAGGCAATCCTGAGGAAGAGGAT |
| | GTGGATACCTCCCAAGTCCTGTATGAGTGGG |
| | AA |
| EGFR-2369CT | GTGGACAACCCCCACGTGTGCCGCCTGCTGG |
| | GCATCTGCCTCACCTCCACCGTGCAGCTCAT |
| | CA[C/T]GCAGCTCATGCCCTTCGGCTGCCT |
| | CCTGGACTATGTCCGGGAACACAAAGACAAT |
| | ATTGGCTCCCAGTACCTGCTCAACTGGTGTG |
| | TGCAGATCGCAAAGGTAATCAGGGAAGGGA |
| EGFR-2573TG | GCATGAACTACTTGGAGGACCGTCGCTTGGT |
| | GCACCGCGACCTGGCAGCCAGGAACGTACTG |
| | GTGAAAACACCGCAGCATGTCAAGATCACAG |
| | ATTTTGGGC[T/G]GGCCAAACTGCTGGGTG |
| | CGGAAGAGAAAGAATACCATGCAGAAGGAGG |
| | CAAAGTAAGGAGGTG |
| KRAS-176CG | CAGGATTCCTACAGGAAGCAAGTAGTAATTG |
| | ATGGAGAAACCTGTCTCTTGGATATTCTCGA |
| | CACAG[C/G]AGGTCAAGAGGAGTACAGTGC |
| | AATGAGGGACCAGTACATGAGGACTGGGGAG |
| | GGCTTTCTTTGTGTATTTGCCATAAATAATA |
| | CTAAATCATTTGAAGATATTC |
| KRAS-183AC | ACAGGAAGCAAGTAGTAATTGATGGAGAAAC |
| | CTGTCTCTTGGATATTCTCGACACAGCAGGT |
| | CA[A/C]GAGGAGTACAGTGCAATGAGGGAC |
| | CAGTACATGAGGACTGGGGAGGGCTTTCTTT |
| | GTGTATTTGCCATAAATAATACTAAATCATT |
| | TGAAGATATTCACCATTATAGGTGGGTTTAA |
| | ATTGAATATAATAAGCTGACATTAA |
| KRAS-34GA | TATTAACCTTATGTGTGACATGTTCTAATAT |
| | AGTCACATTTTCATTATTTTTATTATAAGGC |
| | CTGCTGAAAATGACTGAATATAAACTTGTGG |
| | TAGTTGGAGCT[G/A]GTGGCGTAGGCAAGA |
| | GTGCCTTGACGATACAGCTAATTCAGAATCA |
| | TTTTGTGGACGAATATGA |
| KRAS-35GA | TATTAACCTTATGTGTGACATGTTCTAATAT |
| | AGTCACATTTTCATTATTTTTATTATAAGGC |
| | CTGCTGAAAATGACTGAATATAAACTTGTGG |
| | TAGTTGGAGCTG[G/A]TGGCGTAGGCAAGA |
| | GTGCCTTGACGATACAGCTAATTCAGAATCA |
| | TTTTGTGGACGAATATGATC |
| KRAS-38GA | CATTATTTTTATTATAAGGCCTGCTGAAAAT |
| | GACTGAATATAAACTTGTGGTAGTTGGAGCT |
| | GGTG[G/A]CGTAGGCAAGAGTGCCTTGACG |
| | ATACAGCTAATTCAGAATCATTTTGTGGACG |
| | AATATGATCCAACAATAGAGGTAAATCTTGT |
| | TTTAATATGCATATTACTGGTGCAGGACCAT |
| | TCTTTGATACAGATAAAGGTTTCTCTGACCA |
| | TTTTCATGAGTACTTAT |
| NRAS-181CA | ATTCTTACAGAAAACAAGTGGTTATAGATGG |
| | TGAAACCTGTTTGTTGGACATACTGGATACA |
| | GCTGGA[C/A]AAGAAGAGTACAGTGCCATG |
| | AGAGACCAATACATGAGGACAGGCGAAGGCT |
| | TCCTCTGTGTATTTGCCATCAATAATAGCAA |
| | GTCATTTGCGGATATTAACCTCTACAGGTAC |
| | TAGGAGCATTATTTTCTCTGAAAGGATG |
| NRAS-183AT | TTACAGAAAACAAGTGGTTATAGATGGTGAA |
| | ACCTGTTTGTTGGACATACTGGATACAGCTG |
| | GACA[A/T]GAAGAGTACAGTGCCATGAGAG |
| | ACCAATACATGAGGACAGGCGAAGGCTTCCT |
| | CTGTGTATTTGCCATCAATAATAGCAAGTCA |
| | TTTGCGGATATTAACCTCTACAGGTACTAGG |
| | AGCATTATTTTCTCTGAAAGGATG |
| NRAS-35GA | TGGTTTCCAACAGGTTCTTGCTGGTGTGAAA |
| | TGACTGAGTACAAACTGGTGGTGGTTGGAGC |
| | AG[G/A]TGGTGTTGGGAAAAGCGCACTGAC |
| | AATCCAGCTAATCCAGAACCACTTTGTAGAT |
| | GAATATGATCCCACCATAGAGGTGAGGCCCA |
| | GTGGTAGCCCG |
| NRAS-38GA | TTTCCAACAGGTTCTTGCTGGTGTGAAATGA |
| | CTGAGTACAAACTGGTGGTGGTTGGAGCAGG |
| | TG[G/A]TGTTGGGAAAAGCGCACTGACAAT |
| | CCAGCTAATCCAGAACCACTTTGTAGATGAA |
| | TATGATCCCACCATAGAGGTGAGGCCCAGTG |
| | GTAGCCC |
| TP53-524GA | GGCACCCGCGTCCGCGCCATGGCCATCTACA |
| | AGCAGTCACAGCACATGACGGAGGTTGTGAG |
| | GC[G/A]CTGCCCCCACCATGAGCGCTGCTC |
| | AGATAGCGATGGTGAGCAGCTGGGGCTGGAG |
| | AGACGACAGGGCTGGTTGCCCAGGGTCCCCA |
| | GGCCTCTGATTCCTCACTGATTGCTCTTAGG |
| | TCTGGCC |
| TP53-637CT | CCTCCTCAGCATCTTATCCGAGTGGAAGGAA |
| | ATTTGCGTGTGGAGTATTTGGATGACAGAAA |
| | CACTTTT[C/T]GACATAGTGTGGTGGTGCC |
| | CTATGAGCCGCCTGAGGTCTGGTTTGCAACT |
| | GGGGTCTCTGGGAGGAGGGGTTAAGGGTGGT |
| | TGTCAGTGGCCCTC |
| TP53-721TG | CTTGGGCCTGTGTTATCTCCTAGGTTGGCTC |
| | TGACTGTACCACCATCCACTACAACTACATG |
| | TGTAACAGT[T/G]CCTGCATGGGCGGCATG |
| | AACCGGAGGCCCATCCTCACCATCATCACAC |
| | TGGAAGACTCCAGGTCAGGAGCCACTTGCCA |
| | CCCTGCACACTGGCCTGCTGTGCCCCAGCCTC |
| TP53-733GA | TAGGTTGGCTCTGACTGTACCACCATCCACT |
| | ACAACTACATGTGTAACAGTTCCTGCATGGG |
| | C[G/A]GCATGAACCGGAGGCCCATCCTCAC |
| | CATCATCACACTGGAAGACTCCAGGTCAGGA |
| | GCCACTTGCCACCCTGCACACTGGCCTGCTG |
| | TGCCCCAGCCTC |
| TP53-742CT | CTGACTGTACCACCATCCACTACAACTACAT |
| | GTGTAACAGTTCCTGCATGGGCGGCATGAA |
| | C[C/T]GGAGGCCCATCCTCACCATCATCAC |
| | ACTGGAAGACTCCAGGTCAGGAGCCACTTGC |
| | CACCCTGCACACTGGCCTGCTGTGCCCCAGC |
| | CTCTGCTTGCCTC |
| TP53-743GA | TGACTGTACCACCATCCACTACAACTACATG |
| | TGTAACAGTTCCTGCATGGGCGGCATGAAC |
| | C[G/A]GAGGCCCATCCTCACCATCATCACA |
| | CTGGAAGACTCCAGGTCAGGAGCCACTTGCC |
| | ACCCTGCACACTGGCCTGCTGTGCCCCAGCC |
| | TCTGCTTGCCTC |
| TP53-817CT | CCTCTTGCTTCTCTTTTCCTATCCTGAGTAG |
| | TGGTAATCTACTGGGACGGAACAGCTTTGAG |
| | GTG[C/T]GTGTTTGTGCCTGTCCTGGGAGA |
| | GACCGGCGCACAGAGGAAGAGAATCTCCGCA |
| | AGAAAGGGGAGCCTCACCACGAGCTGCCCCC |
| | AGGGAGCACTAAGCGAGGTAAGCAA |

### Data Analysis

An automatic baseline and manual threshold of 0.2 were used to calculate the threshold cycle (Cₜ) which is defined as the fractional cycle number at which the fluorescence passes the fixed threshold. PCR reactions were run for a total of 50 cycles. For cast-PCR reactions, there was a pre-run of five cycles at a lower annealing/extension temperature followed by an additional 45 cycles at a higher annealing/extension temperature. The ΔCt between amplification reactions for matched vs. mismatched sequences is defined as the specificity of cast-PCR (ΔCt=Ct_{mismatch}−Ct_{match}). The larger the ΔCt between mismatched and matched targets, the better assay specificity. The 2^{ΔCt} value was used to estimate the power of discrimination (or selectivity) which is equal to ½^{ΔCt} or, in some cases, calculated as % (½^{ΔCt}×100).

### Example 1

### Tailed Primers Improve Discrimination of Allelic Variants

The following example demonstrates that the application of allele-specific primers comprising tails significantly improves the discrimination of allelic variants.

In conventional AS-PCR, the discrimination of 3′ nucleotide mismatches is largely dependent on the sequence surrounding the SNP and the nature of the allele. The ΔCt between the amplification reactions for matched and mismatched primers varies. To improve the discrimination between the amplification of matched and mismatched sequences, allele-specific primers were designed to comprise tails at their 5′ termini and then tested for their suitability in AS-PCR assays.

Assays were performed using the general experimental design and reaction conditions indicated above (with the exception that no blocker probes were included and either tailed or non-tailed allele-specific primers were added), using 0.5 ng/uL genomic DNA containing the hsv11711720 SNP comprising one of three alleles (A, C, or T) as the nucleic acid template (see Table 2A). The three genotypes are indicated in Table 2B. Primers and probes were designed according to the sequences shown in Table 3.

TABLE 2A
Genomic DNA Sequence for
hsv11711720 SNP (SEQ ID NO. 28) (target
alleles are indicated in brackets).

AGAAAATAACTAAGGGAAGGAGGAAAGTGGGGAGGAAGGAAGA
ACAGTGTGAAGACAATGGCCTGAAAACTGAAAAAGTCTGTTAA
AGTTAATTATCAGTTTTTGAGTCCAAGAACTGGCTTTGCTACT
TTCTGTAAGTTTCTAATTTACTGAATAAGCATGAAAAAGATTG
CTTTGAGGAATGGTTATAAACACATTCTTAGAGCATAGTAAGC
AGTAGGGAGTAACAAAATAACACTGATTAGAATACTTTACTCT
ACTTAATTAATCAATCATATTTAGTTTGACTCACCTTCCCAG
[A/C/T]ACCTTCTAGTTCTTTCTTATCTTTCAGTGCTTGTCC
AGACAACATTTTCATTTCAACAACTCCTGCTATTGCAATGATG
GGTACAATTGCTAAGAGTAACAGTGTTAGTTGCCAACCATAGA
TGAAGGATATAATTATTCCTGTCCCAAGATTTGCTATATTCTG
GGTAATTACAGCAAGCCTGGAACCTATAGCCTGCAAAACAAAA
CAAATTAGAGAAATTTTAAAAATATTATCTTCACAACTCATGC
TTCTATTTTCTGAAAACTCACCTTCATGAGACTATATTCATTA
TTTTAT

TABLE 2B
Genotypes of Genomic DNA
Sequence for hsv11711720 SNP

| Genomic DNA ID | Genotype |
|---|---|
| NA17203 | AA |
| NA17129 | CC |
| NA17201 | TT |

TABLE 3
List and Sequences of Primers and Probes
(SEQ ID NOS 29-36,respectively, in order
of appearance) for genomic DNA:
conventional allele-
specific primers (“ASP”); tailed allele-
specific primers (“tailASP”); locus-specific
TaqMan probe (LST); locus-specific primer
(LSP). The nucleotides shown in lower
case are the tailed portion
of the primers. The nucleotide-
specific portion of each allele-
specific primer is at the 3'-most terminus
of each primer (indicated in bold).

| Primer/ | | Tm |
|---|---|---|
| Probe ID | Sequence (5′ to 3′) | (° C.) |
| 17129-ASP | ATATTTAGTTTGACTCACCTTCCCAGC | 63.2 |
| 17129- | accACTCACCTTTCCCAGC | 63.0 |
| tailASP | | |
| 17203-ASP | ATATTTAGTTTGACTCACCTTCCCAGA | 62.0 |
| 17203- | accACTCACCTTTCCCAGA | 63.7 |
| tailASP | | |
| 17201-ASP | ATATTTAGTTTGACTCACCTTCCCAGT | 62.2 |
| 17201- | accACTCACCTTTCCCAGT | 64.0 |
| tailASP | | |
| LST | (6-FAM)-TGGACAAGCACTGAAAGA-(MGB) | 67.4 |
| LSP | GCAGGAGTTGTTGAAATGAAAATGTTG | 62.5 |

As shown in Table 4, when using non-tailed ASPs (“ASP−tail”), the discrimination of 3′ nucleotide mismatch is largely dependent on the nature of the allele, as a considerable range of ΔCt values is observed depending on the identity of the 3′-terminal base. The range of ΔCt values between matched and mismatched nucleotides (“NT”) were from −0.1 to 10. However, with tailed ASPs (ASP+tail), the discrimination of 3′ nucleotide mismatch was significantly improved. In fact, as Table 4 shows, the ΔCt value between matched and mismatched nucleotides was consistently equal to or greater than 10 when tailed ASPs were used. The Ct values for amplification of matched sequences using tailed ASPs were comparable to those using conventional or non-tailed ASPs. These results indicate that tailed ASP, can improve the specificity of AS-PCR, but may not improve the sensitivity of detection.

TABLE 4
Tailed allele-specific primers (“ASP”) significantly
improve discrimination of allelic variants.
The specificity (“fold difference”) was calculated based
on the difference between Ct values using tailed vs.
untailed primers (2^{(ΔCt(ASP +} ^{tail) -} ^{(ΔCt(ASP-tail)}). The mismatched
nucleotides of the 3′ allele-specific nucleotide portion
of the ASPs (+/−tail) are also indicated (“NT mismatch”).

| NT | ΔCt | ΔCt | Specificity Improvement |
|---|---|---|---|
| mismatch | (ASP − tail) | (ASP + tail) | (fold difference) |
| C-A | 0.9 | 11.5 | 1552.1 |
| C-T | 1.2 | 11.5 | 1278.3 |
| A-C | 10.0 | 11.9 | 3.7 |
| A-G | 9.8 | 11.9 | 4.3 |
| T-G | 2.3 | 11.5 | 588.1 |
| T-C | −0.1 | 11.5 | 3104.2 |
| Average | 4.0 | 11.6 | 1088.5 |

### Example 2

### Low Primer Concentrations Improve Discrimination of Allelic Variants

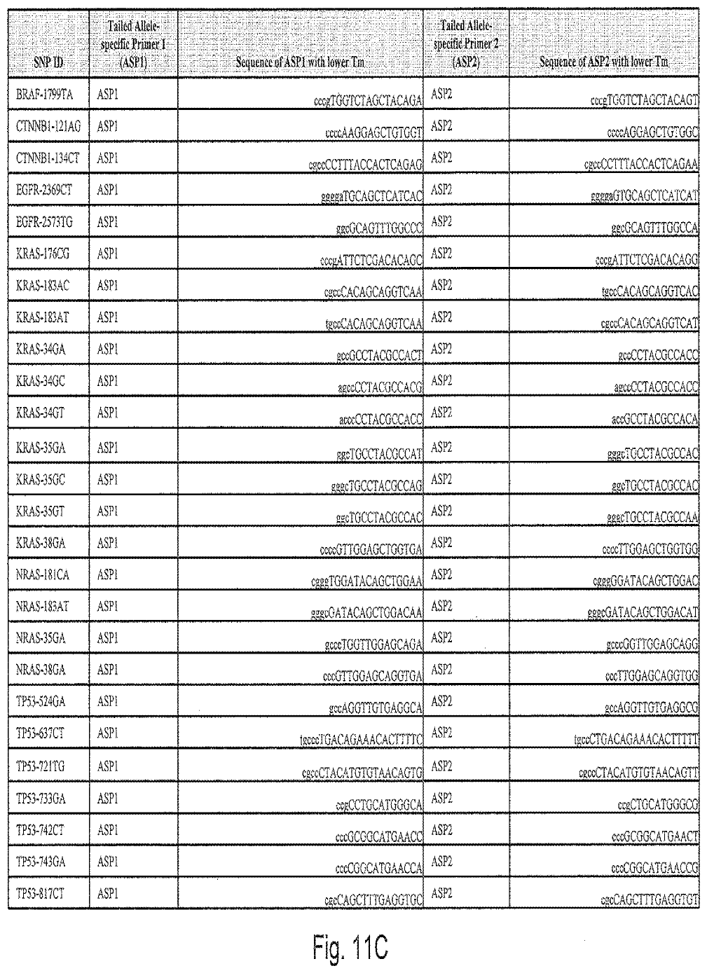

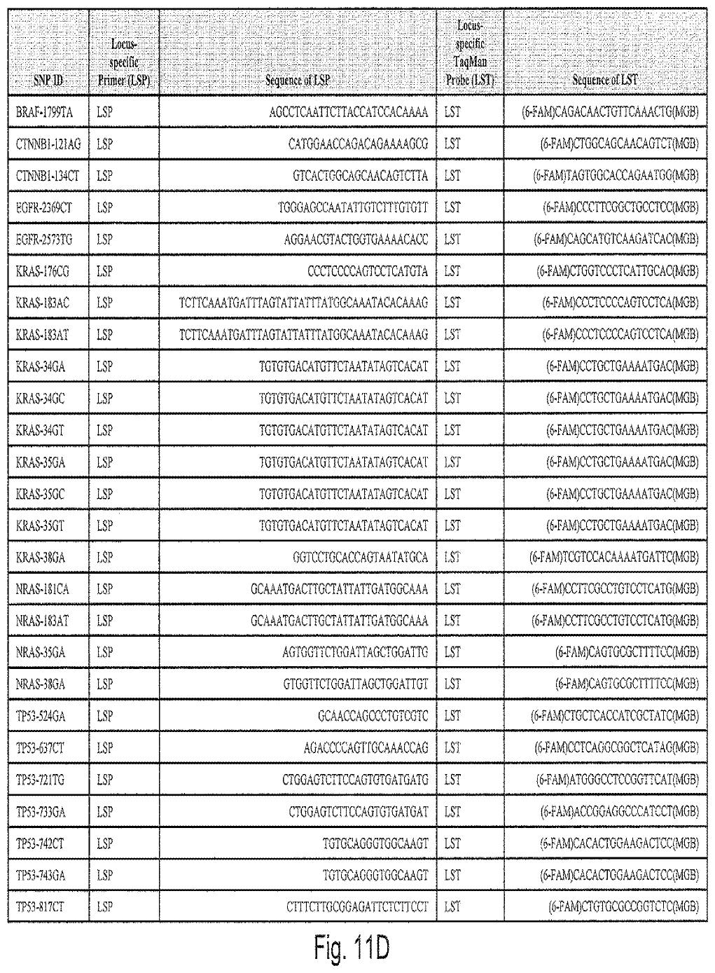

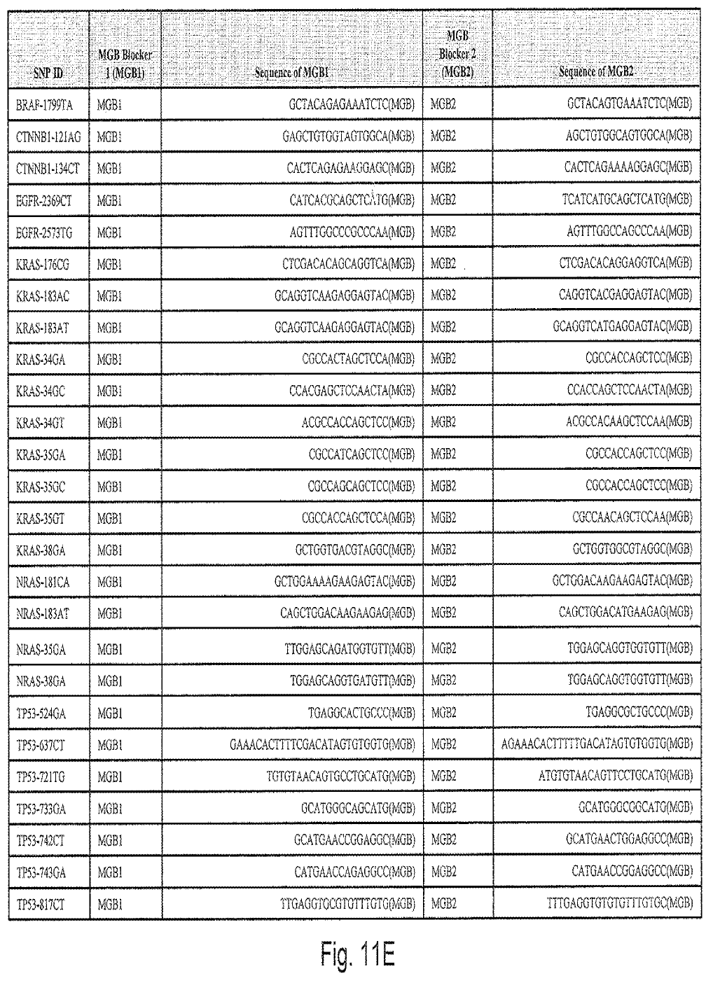

Assays were performed using the general experimental design and reaction conditions indicated above, in the presence of 1 million copies of plasmid DNA containing various SNP target sequences (see Table 1) and 200 nM or 800 nM tailed ASP (as indicated). Assay primers and probes were designed according to the sequences shown in FIG. 11A.

The effect of tailed ASP concentration on discrimination of allelic variants is summarized in Table 5. The ΔCt between the amplification reactions for matched and mismatched primers demonstrate that lower tailed ASP concentrations improve discrimination of allelic variants.

TABLE 5
Assay Results Using Different Concentrations
of Tailed Allele-specific Primers

| | ΔCt | ΔCt | Specificity |
|---|---|---|---|
| Plasmid | (ASP @ | (ASP @ | Improvement |
| SNP ID | 800 nM) | 200 nM) | (fold difference) |
| CV11201742 | 14.1 | 15.2 | 2.14 |
| CV11349123 | 8.2 | 10 | 3.48 |
| CV1207700 | 5.2 | 6.6 | 2.64 |
| CV25594064 | 20.1 | 19.1 | 0.5 |
| CV25639181 | 11.9 | 12.9 | 2 |
| Average | 12.6 | 13.44 | 2.14 |

### Example 3

### Primers Designed with Reduced Tms Improves Discrimination of Allelic Variants

Assays were performed using the general experimental design and reaction conditions indicated above, in the presence of 1 million copies of plasmid DNA containing various SNP target sequences (see Table 1) using tailed ASP with a higher Tm (˜57° C.) or tailed ASP with a lower Tm (˜53° C.). Assay primers and probes were designed according to the sequences shown in FIG. 11B-E (see FIG. 11B for higher Tm ASP and FIG. 11C or lower Tm ASP).

The effect of allele-specific primer Tm on discrimination of allelic variants is summarized in Table 6. The ΔCt of allele-specific primers with a lower Tm are significantly higher than those of allele-specific primers with a higher Tm. Allele-specific primers designed with reduced Tms improved discrimination of allelic variants by as much as 118-fold in some cases or an average of about 13-fold difference.

TABLE 6
ΔCt Values Using Tailed ΔSPs with Lower Tm (~53° C.)
or with Higher Tm (~57° C.)

| | | | Specificity |
|---|---|---|---|
| Plasmid | ΔCt (ASP w/ | ΔCt (ASP w/ | Improvement |
| SNP ID | Tm ~57° C.) | Tm ~53° C.) | (fold difference) |
| BRAF-1799TA | 12.2 | 19.1 | 118.9 |
| CTNNB1-121AG | 11.6 | 14.9 | 10.0 |
| KRAS-176CG | 18.8 | 22.5 | 13.1 |
| NRAS-35GA | 13.0 | 14.0 | 1 |
| TP53-721TG | 14.7 | 19.1 | 20.6 |
| CTNNB1-134CT | 8.6 | 14.1 | 44.8 |
| EGFR-2369CT | 9.7 | 10.7 | 2 |
| KRAS-183AC | 22.2 | 23.1 | 1.8 |
| NRAS-38GA | 14.0 | 14.3 | 1.2 |
| TP53-733GA | 13.6 | 13.5 | 1.0 |
| EGFR-2573TG | 16.7 | 20.2 | 10.9 |
| KRAS-34GA | 14 | 14.8 | 1.8 |
| KRAS-38GA | 11.2 | 14.4 | 8.9 |
| S-181CA | 24.0 | 27.1 | 8.6 |
| TP53-742CT | 9.1 | 8.0 | 0.5 |
| KRAS-35GA | 11.5 | 15.1 | 12.3 |
| NRAS-183AT | 23.6 | 22.7 | 0.5 |
| TP53-524GA | 11.4 | 13.5 | 4.6 |
| TP53-637CT | 11.4 | 14.4 | 7.8 |
| TP53-743GA | 10.1 | 13.2 | 8.4 |
| TP53-817CT | 13.6 | 13.9 | 1.2 |
| Average | 14.1 | 16.3 | 13.3 |

### Example 4

### Use of Blocker Probes Improves Discrimination of Allelic Variants

The following example illustrates that the use of MGB blocker probes improves the discrimination between 3′ nucleotide mismatched and matched primers to target sequences in AS-PCR reactions.

Assays were performed using the general cast-PCR schema and reaction conditions indicated above, using 1 million copies of plasmid DNA containing various SNP target sequences (see Table 1) in the presence of MGB blocker probes or in the absence of MGB blocker probes. Assay primers and probes were designed according to the sequences shown in FIG. 11C-E.

To improve the selectivity of AS-PCR, blocker probes were synthesized to comprise an MGB group at their 3′ terminus. (See, for example, Kutyavin, I. V., et al., Nucleic Acids Research, 2000, Vol. 28, No. 2: 655-661, U.S. Pat. Nos. 5,512,677; 5,419,966; 5,696,251; 5,585,481; 5,942,610 and 5,736,626.)

The results of cast-PCR using MGB blocker probes are summarized in Table 7. The ΔCt between cast-PCR with MGB blocker probes is larger than that without MGB blocker probes. As shown, MGB blocker probes improve the discrimination of allelic variants.

TABLE 7
MGB Blocker Probes Improve Discrimination of Allelic Variants

| | ΔCt (no | ΔCt | Specificity |
|---|---|---|---|
| | MGB | (+MGB | Improvement |
| SNP ID | blocker) | blocker) | (fold difference) |
| BRAF-1799TA | 11.4 | 14.9 | 11.5 |
| CTNNB1-121AG | 11.6 | 14.1 | 5.4 |
| KRAS-176CG | 17.8 | 20.9 | 9 |
| NRAS-35GA | 13.9 | 14.3 | 1.4 |
| TP53-721TG | 12.5 | 14.7 | 4.4 |
| CTNNB1-134CT | 6.7 | 10.2 | 11.6 |
| EGFR-2369CT | 7.7 | 10.1 | 5.3 |
| KRAS-183AC | 22.4 | 23 | 1.5 |
| NRAS-38GA | 14.5 | 14.6 | 1.1 |
| TP53-733GA | 13.2 | 14.4 | 2.3 |
| EGFR-2573TG | 18.2 | 21.8 | 11.6 |
| KRAS-34GA | 14.4 | 15.1 | 1.7 |
| KRAS-38GA | 11.9 | 15.1 | 1.7 |
| NRAS-181CA | 19.3 | 24.2 | 30.2 |
| TP53-742CT | 12.7 | 13.6 | 1.9 |
| KRAS-35GA | 11.0 | 13.7 | 6.5 |
| NRAS-183AT | 20.2 | 21.7 | 2.9 |
| TP53-524GA | 13.5 | 13.5 | 1 |
| TP53-637CT | 9.3 | 12.1 | 7.0 |
| TP53-743GA | 9.9 | 11.5 | 3.1 |
| TP53-817CT | 12.6 | 13.2 | 1.5 |
| Average | 13.6 | 15.5 | 6.0 |

### Example 5

### Primers Designed to Target Discriminating Bases Improves Discrimination of Allelic Variants

Assays were performed using the general cast-PCR schema and reaction conditions indicated above, in the presence of 1 million copies of plasmid DNA containing SNP target sequences (see Table 1). Assay primers and probes were designed according to the sequences shown in FIG. 11C-E.

According to the data summarized in Table 8, the discrimination of cast-PCR was dependent on the nature of the allele being analyzed. As Table 8 indicates, the ΔCt between mismatched and matched sequences for allele-1 were different from ΔCt between mismatched and matched sequences for allele-2. However, both A and G bases, as compared to a T base, were highly discriminating for allele-1 and allele-2 in all four SNPs examined.

TABLE 8
Primers Designed to Target Discriminating Bases Improve Discrimination of Allelic Variants

| | ASP design | | SNP allele-1 | | | SNP allele-2 | | |
|---|---|---|---|---|---|---|---|---|
| | 3′ NT | 3′ NT | | ΔCt | Specificity | | ΔCt | Specificity |
| | of | of | Allele | (Ct_mismatch − | (fold | Allele | (Ct_mismatch − | (fold |
| SNP ID | ASP1 | ASP2 | NT | Ct_match) | difference) | NT | Ct_match) | difference) |
| KRAS-38GA | G | A | C | 13.4 | 10809 | T | 8.2 | 294 |
| NRAS-181CA | C | A | G | 27.5 | 189812531 | T | 9.8 | 891 |
| NRAS-183AT | A | T | T | 17.9 | 244589 | A | 23.4 | 11068835 |
| TP53-742CT | C | T | G | 12.3 | 5043 | A | 8.3 | 315 |

### Example 6

### Determination of the Sensitivity and Dynamic Range for Cast-PCR

In this example, the sensitivity and dynamic range of cast-PCR was determined by performing cast-PCR using various copy numbers of a target plasmid.

Assays were performed using the general cast-PCR schema and reaction conditions indicated above, using 1×10⁰ (1 copy) to 1×10⁷ copies of plasmid DNA containing the NRAS-181CA SNP target sequence (see Table 1). Assay primers and probes were designed according to the sequences shown in FIG. 11C-E.

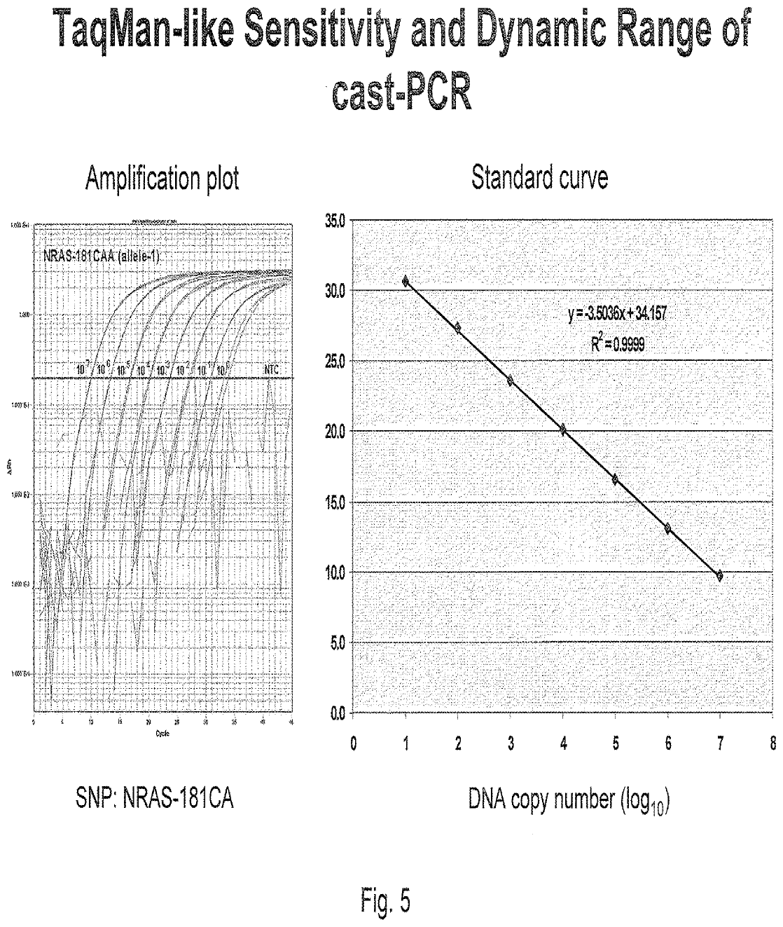

As shown in FIG. 5, the use of tailed primers and MGB-blocker probes does not adversely affect the sensitivity of cast-PCR, as the sensitivity of cast-PCR is comparable to TaqMan assays which do not utilize tailed primers or blocker probes. Furthermore, FIG. 5 shows that the cast-PCR assay shows a linear dynamic range over at least 7 logs.

### Example 7

### Determination of the Specificity of Cast-PCR

In this example, the specificity of cast-PCR was determined by comparing the amplification of particular alleles of KRAS using either matched or mismatched ASPs to a given allele in the presence of their corresponding blocker probes.

Assays were performed using the general cast-PCR schema and reaction conditions indicated above, using 1×10⁶ copies of plasmid DNA containing either one of two alleles of the KRAS-183AC SNP target sequence (see Table 1). Assay primers and probes were designed according to the sequences shown in FIG. 11C-E.

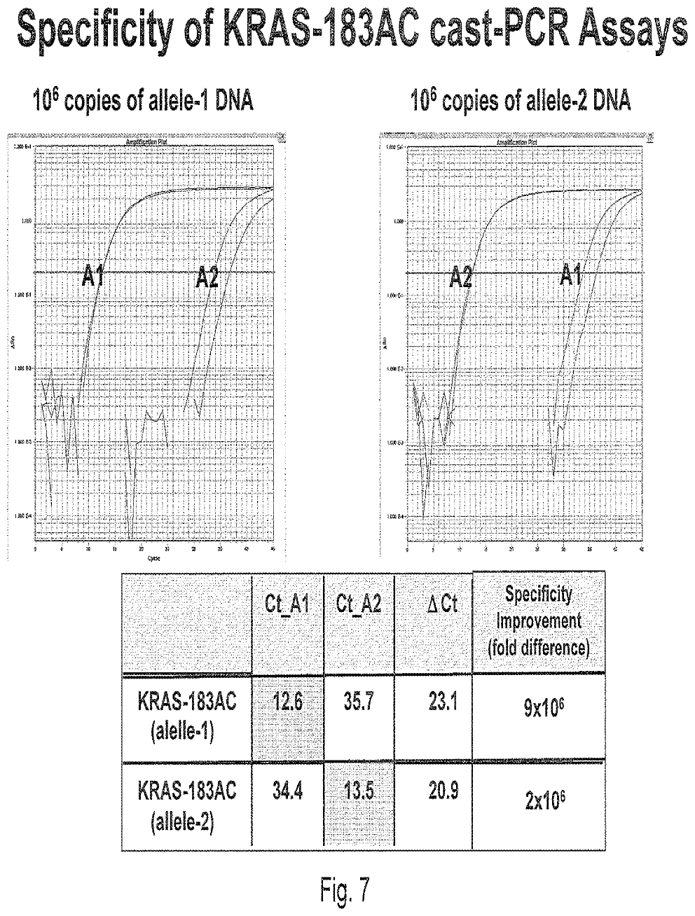

The left panel of FIG. 7 shows the an amplification plot of cast-PCR on allele-1 DNA using matched (A1) primers in the presence of A2 blocker probes or mismatched (A2) primers in the presence of A1 blocker probes. The right hand panel shows a similar experiment in which cast-PCR was performed on allele-2 DNA. As indicated in the data summary in FIG. 7, a robust ΔCt values of over 20 were observed for cast-PCR on both alleles of KRAS-183AC tested. This corresponds to a specificity as determined by a calculation of 2^{ΔCt} of 9×10⁶, and 2×10⁶, respectively, for allele-1 and allele-2. Furthermore, a calculation of selectivity (½^{ΔCt}) indicates that values of 1/1.1×10⁷ and 1/5.0×10⁷ are observed for allele-1 and allele-2, respectively.

### Example 8

### Cast-PCR is Able to Detect a Single Copy Mutant DNA in One Million Copies of Wild Type DNA

In this example, the selectivity of cast-PCR, i.e., the ability of cast-PCR to detect a rare mutant DNA in an excess of wild type DNA, was determined.

Assays were performed using the general cast-PCR schema and reaction conditions indicated above, using various copy numbers of mutant KRAS-183AC plasmid DNA (1 copy to 1×10⁶ copies) mixed with 1×10⁶ copies of wild type KRAS-183AC plasmid DNA (see Table 1). Assay primers and probes were designed according to the sequences shown in FIG. 11C-E, and cast-PCR reactions were performed using wild type or mutant allele-specific primers and the corresponding MGB blocker probes.

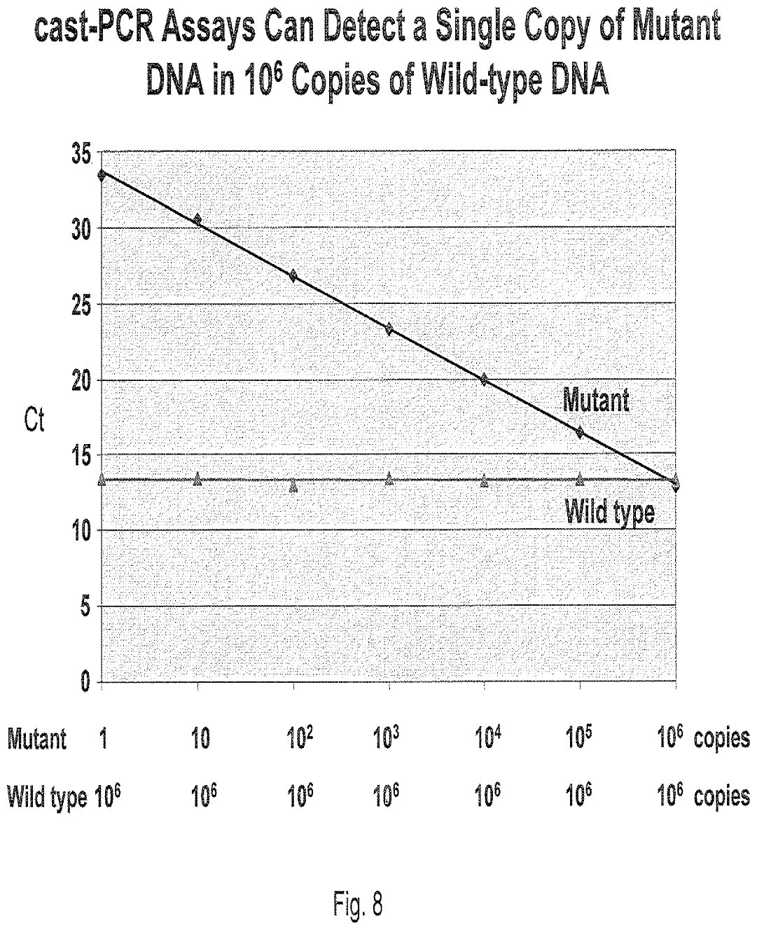

FIG. 8 shows that cast-PCR is able to detect as little as one copy of a mutant DNA sequence, even when surrounded by a million-fold excess of a wild type sequence.

### Example 9

### Selectivity of Cast-PCR in Discriminating Tumor Cell DNA from Normal Cell DNA

In this example, the selectivity of cast-PCR was determined by performing assays on samples in which various amounts of tumor cell genomic DNA were mixed with or “spiked” into genomic DNA from normal cells. DNA samples were extracted using QIAmp DNA Mini Prep Kits (Qiagen). Wild type DNA was extracted from the SW48 cell line and mutant DNA was extracted the H1573 cell line.

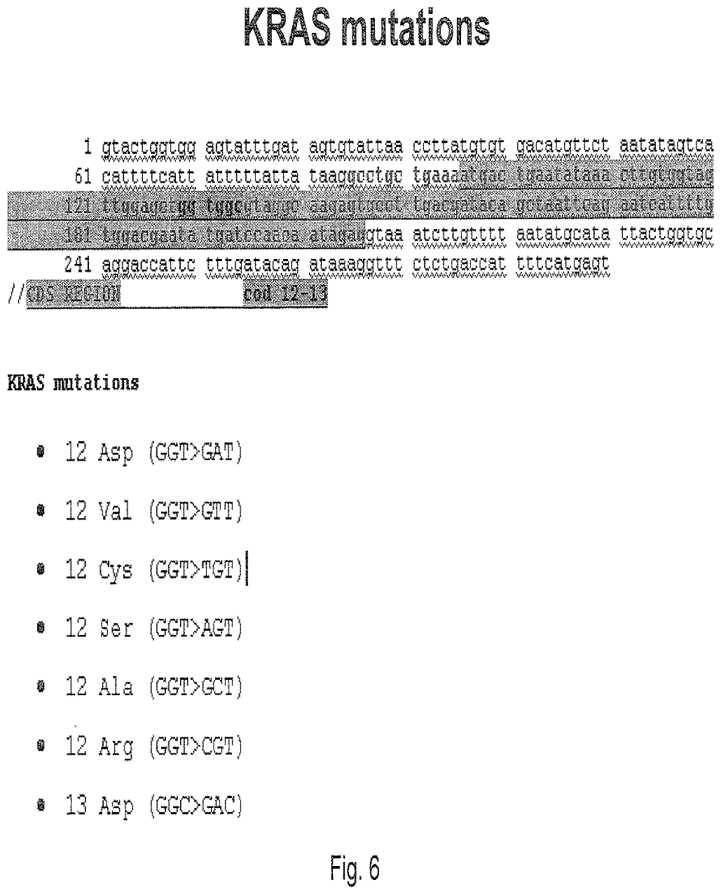

The mutant DNA contained the KRAS-G12A mutation (See FIG. 6). The percentage of tumor cell DNA in the spiked samples varied from 0.5 to 100%. cast-PCR was used to detect the presence of tumor cell DNA when present in these percentages.

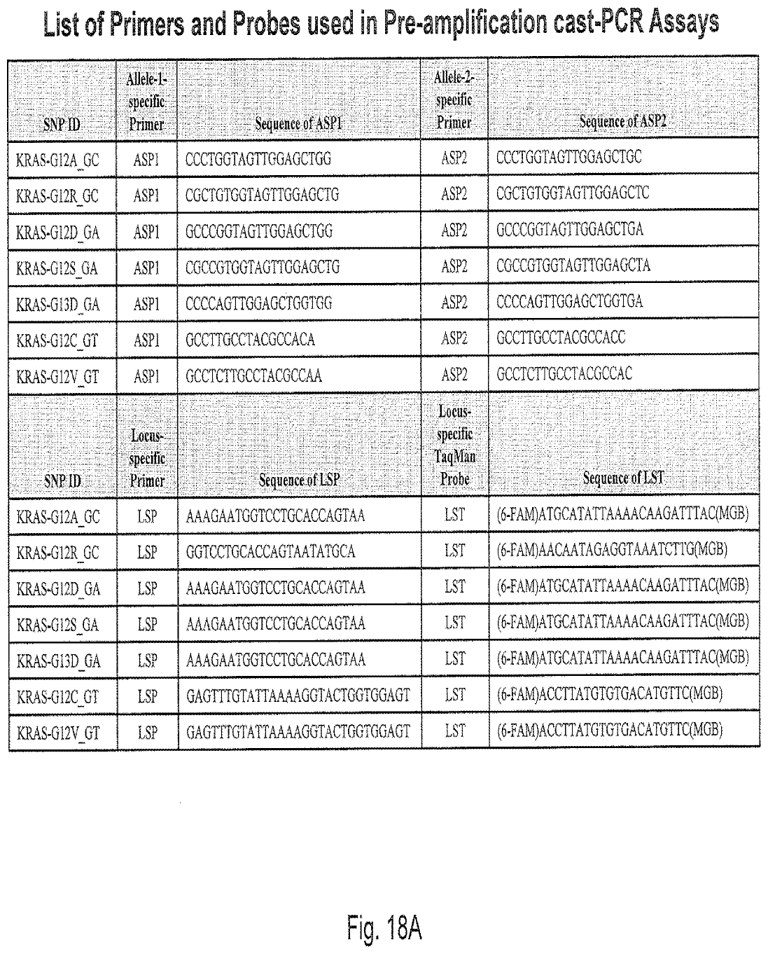

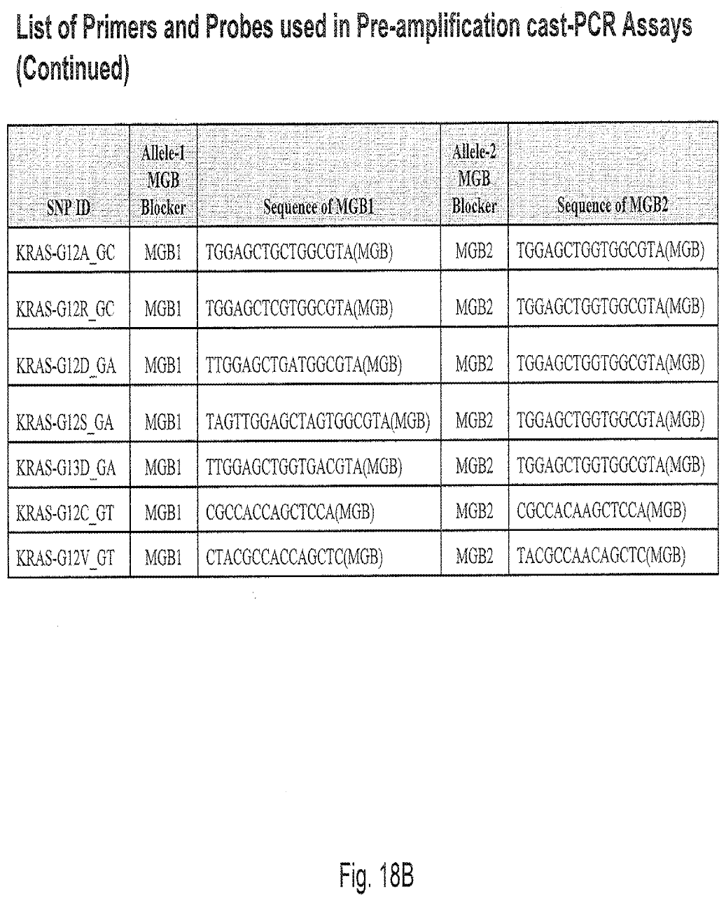

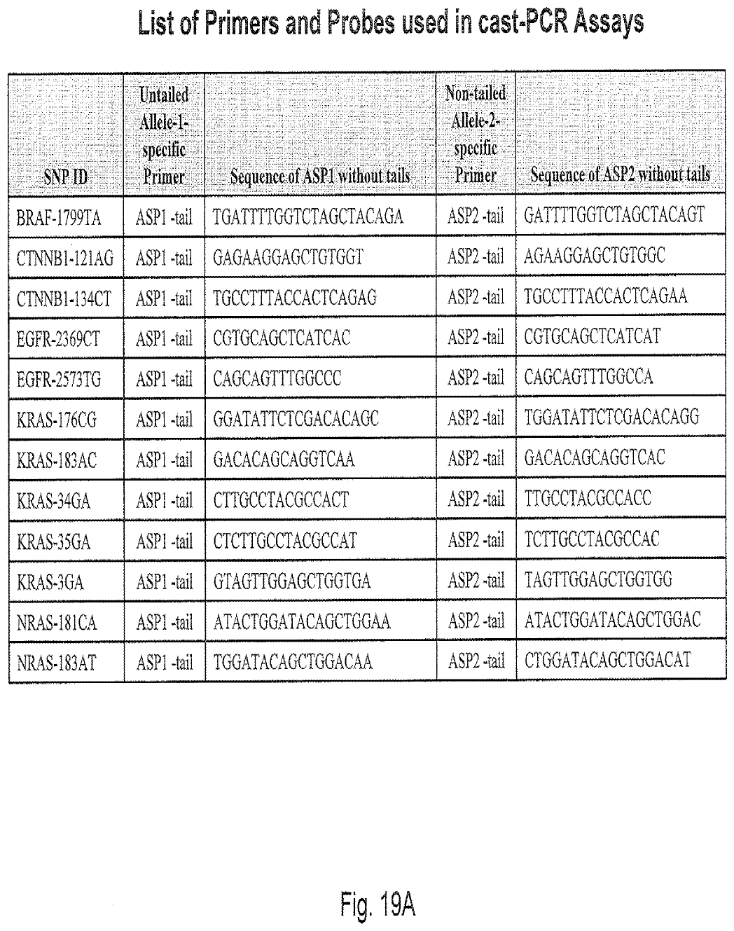

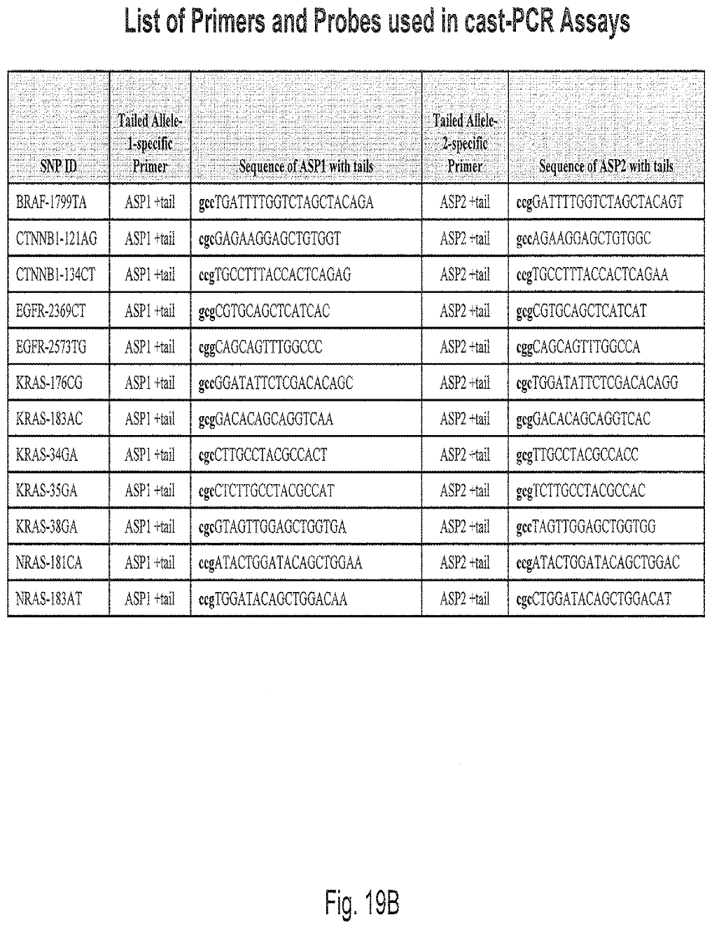

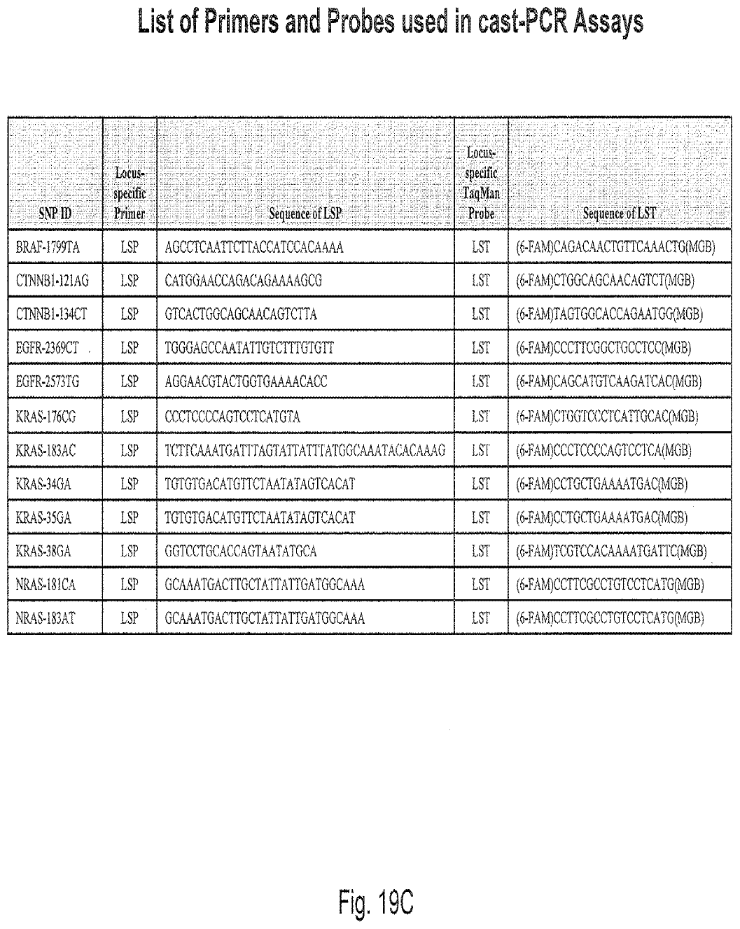

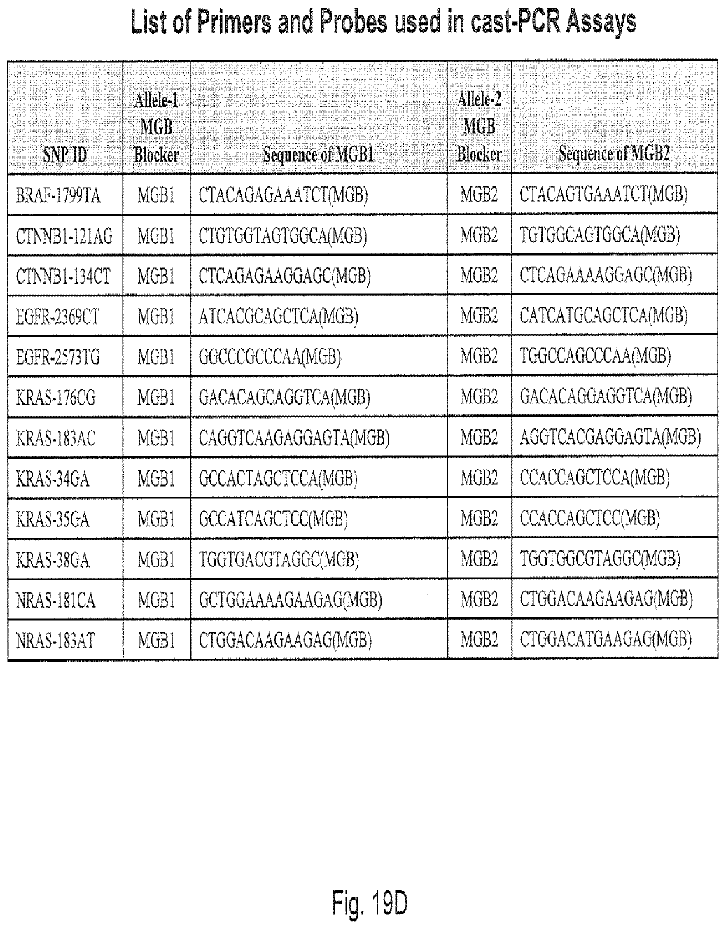

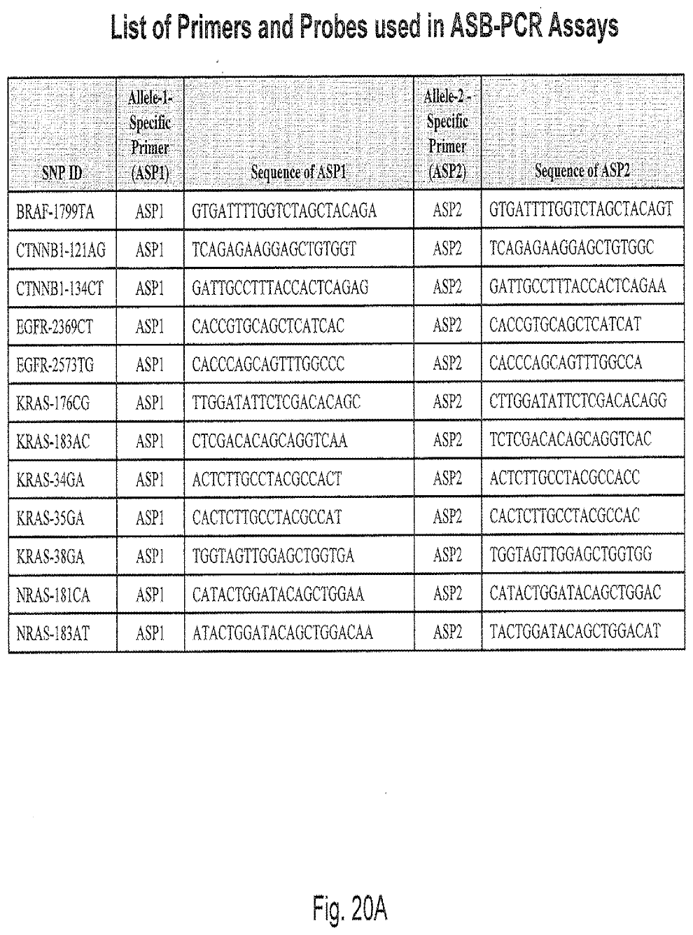

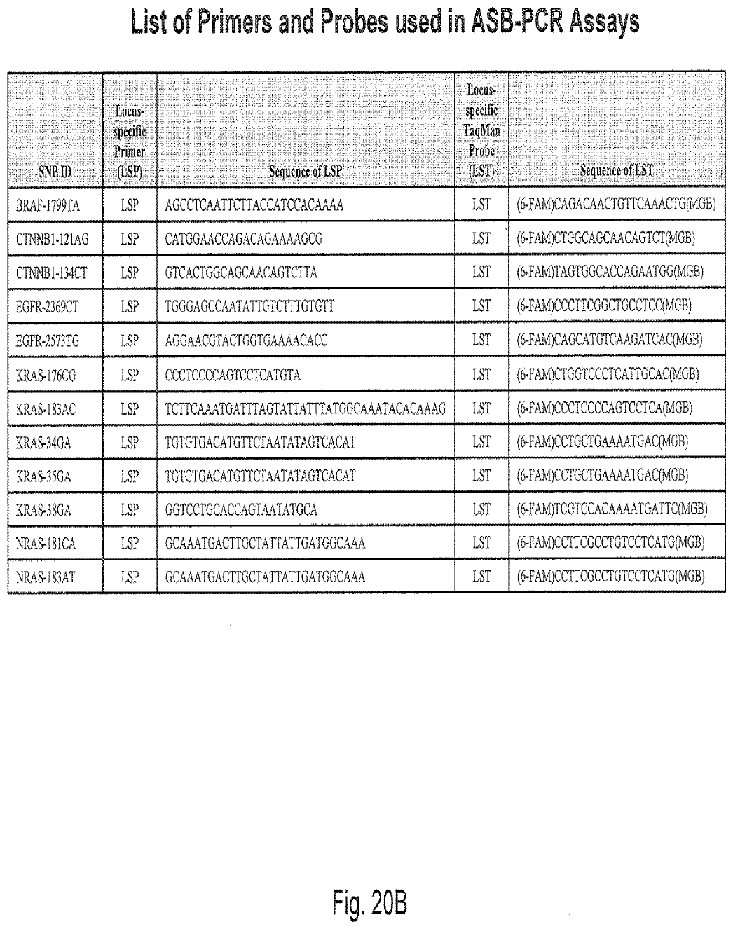

Assays were performed using the general cast-PCR schema and reaction conditions indicated above, using 30 ng of gDNA per reaction. Assay primers and probes were designed according to the sequences corresponding to KRAS-G12A SNP ID, as shown in FIGS. 18A and 18B.

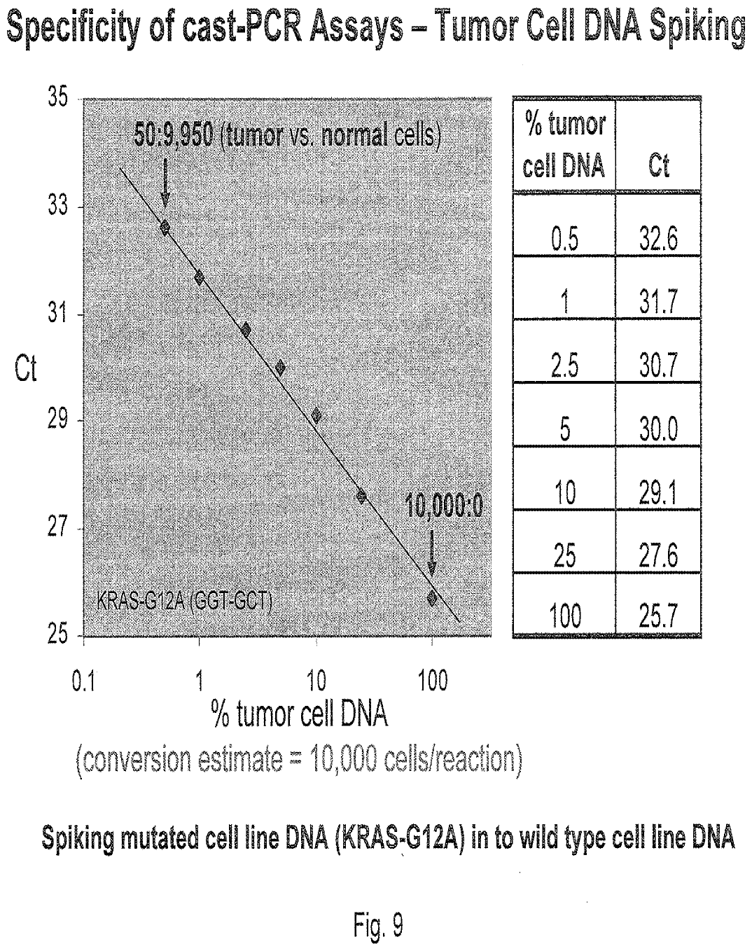

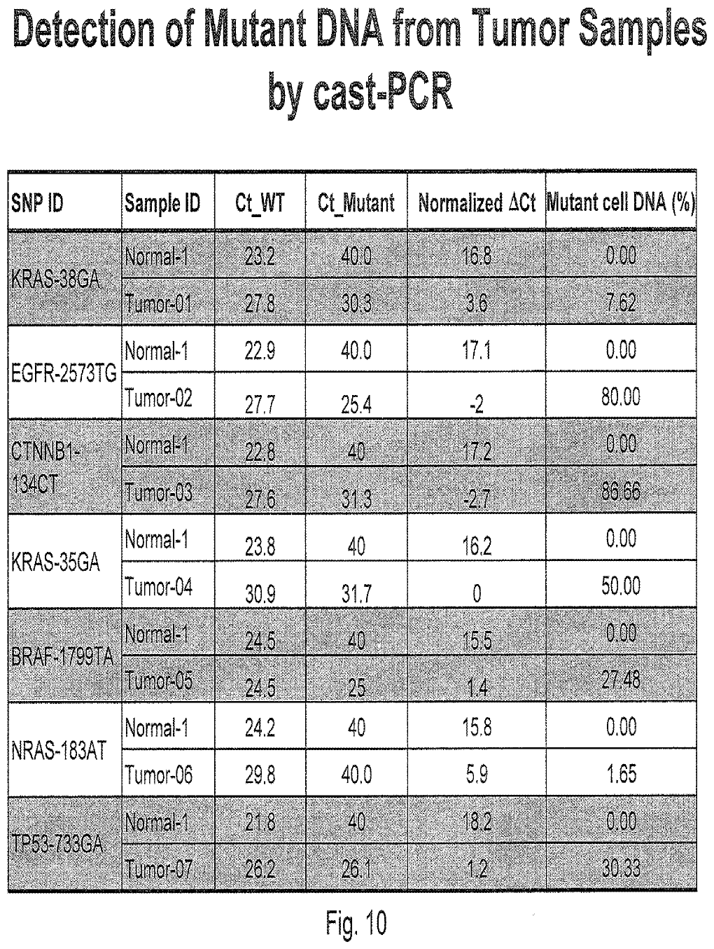

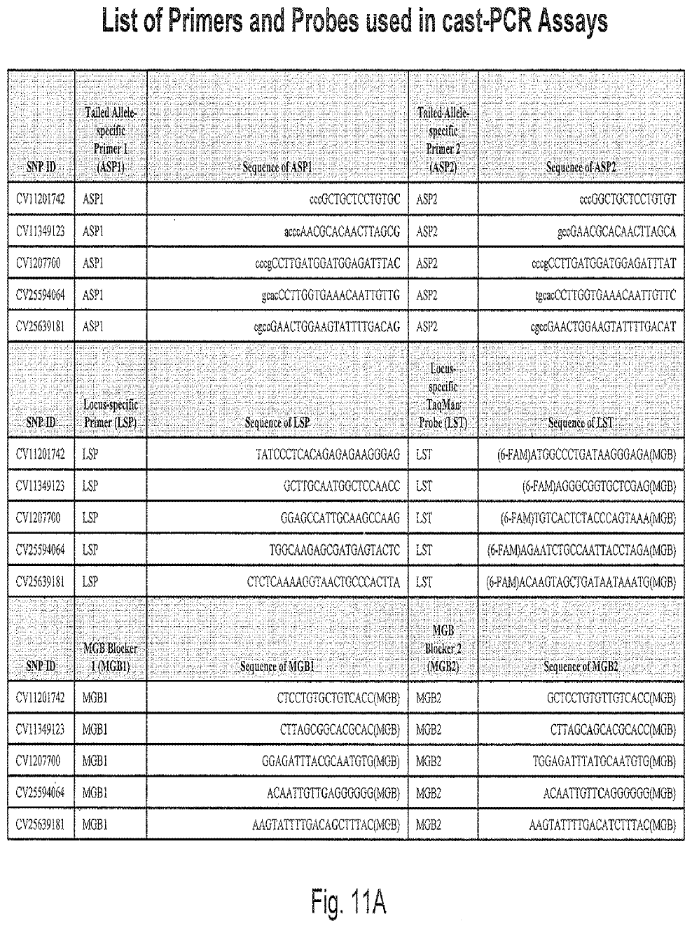

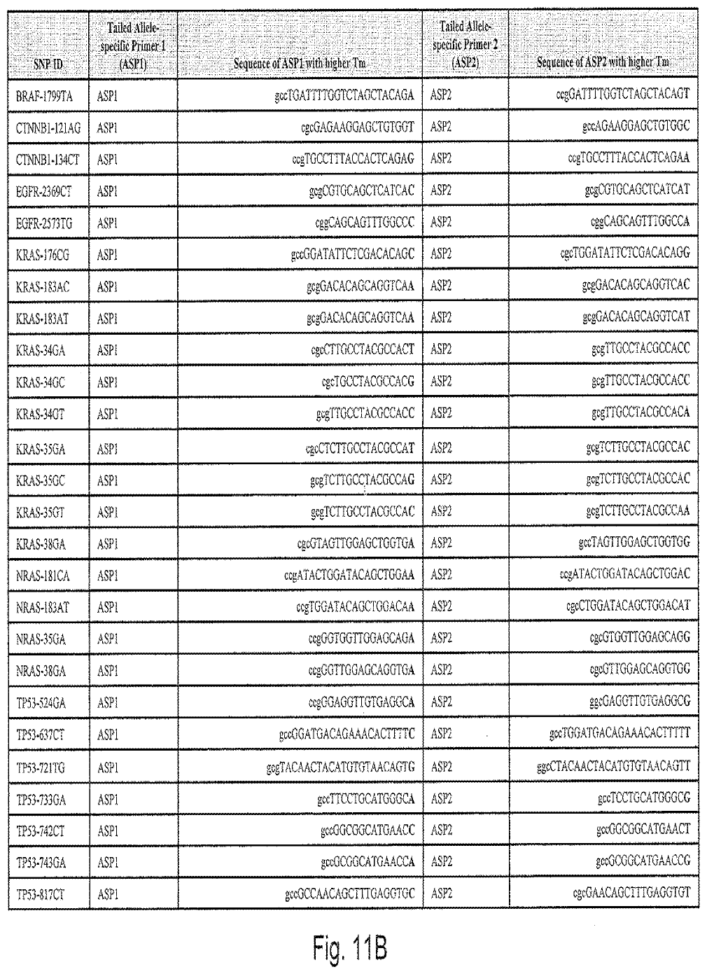

As shown in FIG. 9, tumor cell DNA, even when present only at a level of 0.5% as compared to normal cell DNA, is easily detected using cast-PCR.

### Example 10

### Use of Cast-PCR to Detect Tumor Cells in Tumor Samples

In this example, cast-PCR was used to detect and determine the percentage of tumor cells in tumor samples. Various normal and tumor samples were obtained and assayed by cast-PCR for the presence of a number of SNPs associated with cancer as shown in FIG. 10.

Assays were performed using the general cast-PCR schema and reaction conditions indicated above, using 5 ng of gDNA or 1.5 ng cDNA derived from either normal or tumor samples. Assay primers and probes corresponding to the SNPs shown in FIG. 10 were designed according to the sequences as shown in FIG. 11C-E.

The results shown in FIG. 10 indicate that cast-PCR has a low false positive rate as indicated by the failure of cast-PCR to detect the presence of mutant cells in normal samples. In contrast, cast-PCR was able to provide a determination of the percentage of tumor cells in various tumor samples that ranged from just under 2% for a tumor sample containing the NRAS-183AT SNP to greater than 80% for a sample containing the CTNNB1-134CT SNP.

### Example 11

### Use of Pre-Amplification in Cast-PCR

The following example demonstrates that pre-amplification combined with cast-PCR methods enables detection of multiple alleles from a limited amount of genomic DNA template.

Prior to conducting cast-PCR amplification, multiplex reactions were performed for 7 different KRAS mutations (see Table 9).

10 μl multiplex reactions were prepared in a single tube by combining into one reaction 45 nM of each allele-specific primer (including the allele-1-speicific primer and the allele-2-specific primer) and 45 nM of each locus-specific primer for each of the seven different KRAS SNPs, as listed in FIGS. 18A and 18B, 0.1 ng/μL genomic DNA, and 1× Preamp Master Mix (Applied Biosystems, Foster City, CA; P/N 437135). The 10 μl pre-amplification reactions were then incubated in an Applied Biosystems 9700 Thermocyler in a 96- or 384-well plate for 95° C. for 10 minutes, followed by 10 cycles of 95° C. for 15 seconds, 60° C. for 4 minutes, and 99.9° C. for 10 minutes, and then held at 4° C. Next, 190 μl of 0.1× TE pH 8.0 was added to each 10 μl pre-amplification reaction (20× dilution). The diluted pre-amplification reaction products were then directly used in subsequent cast-PCR reactions or stored at −20° C. for at least one week prior to use.

TABLE 9
KRAS SNP Sequences (SEQ ID NOS 37-42,
respectively, in order of
appearance) (target alleles are
indicated in brackets).

| SNP ID | SNP Sequence |
|---|---|
| KRAS-G12A_GC | TGACTGAATATAAACTTGTGGTAGTTGGAG |
| | CTG[G/C]TGGCGTAGGCAAGAGTGCCTTG |
| | ACGATACAGCTAATTCAGAATCATTTTGTG |
| | GACGAATATGATCCAACAATAGAGGTAAAT |
| | CTTGTTTTAATATGCATATTACTGGTGCAG |
| | GACCATTCTTTGATACA |
| KRAS-G12R_GC | TGACTGAATATAAACTTGTGGTAGTTGGAG |
| | CT[G/C]GTGGCGTAGGCAAGAGTGCCTTG |
| | ACGATACAGCTAATTCAGAATCATTTTGTG |
| | GACGAATATGATCCAACAATAGAGGTAAAT |
| | CTTGTTTTAATATGCATATTACTGGTGCAG |
| | GACCATTCTTTGATACA |
| KRAS-G12D_GA | TGACTGAATATAAACTTGTGGTAGTTGGAG |
| | CTG[G/A]TGGCGTAGGCAAGAGTGCCTTG |
| | ACGATACAGCTAATTCAGAATCATTTTGTG |
| | GACGAATATGATCCAACAATAGAGGTAAAT |
| | CTTGTTTTAATATGCATATTACTGGTGCAG |
| | GACCATTCTTTGATACA |
| KRAS-G12S_GA | TGACTGAATATAAACTTGTGGTAGTTGGAG |
| | CT[G/A]GTGGCGTAGGCAAGAGTGCCTTG |
| | ACGATACAGCTAATTCAGAATCATTTTGTG |
| | GACGAATATGATCCAACAATAGAGGTAAAT |
| | CTTGTTTTAATATGCATATTACTGGTGCAG |
| | GACCATTCTTTGATACA |
| KRAS-G13D_GA | TGACTGAATATAAACTTGTGGTAGTTGGAG |
| | CTGGTG[G/A]CGTAGGCAAGAGTGCCTTG |
| | ACGATACAGCTAATTCAGAATCATTTTGTG |
| | GACGAATATGATCCAACAATAGAGGTAAAT |
| | CTTGTTTTAATATGCATATTACTGGTGCAG |
| | GACCATTCTTTGATACA |
| KRAS-G12C_GT | GTGAGTTTGTATTAAAAGGTACTGGTGGAG |
| | TATNNGATAGTGTATTAACCTTATGTGTGA |
| | CATGTTCTAATATAGTCACATTTTCATTAT |
| | TTTTATTATAAGGCCTGCTGAAAATGACTG |
| | AATATAAACTTGTGGTAGTTGGAGCT[G/T] |
| | GTGGCGTAGGCAAGAGT |

Following pre-amplification, the diluted pre-amplification products were aliquotted into single-plex cast-PCR reactions. Individual assays were performed for each of the 7 different KRAS mutations using the general experimental design and reaction conditions indicated above (see section II of Examples). 10 μL cast-PCR reactions were run for assays containing, as the nucleic acid template, either 1 μL 20× diluted pre-amplification reaction product (as prepared above) or, as a comparison, 0.07 ng genomic DNA. All assay primers and probes were designed according to the sequences shown in FIGS. 18A and 18B.

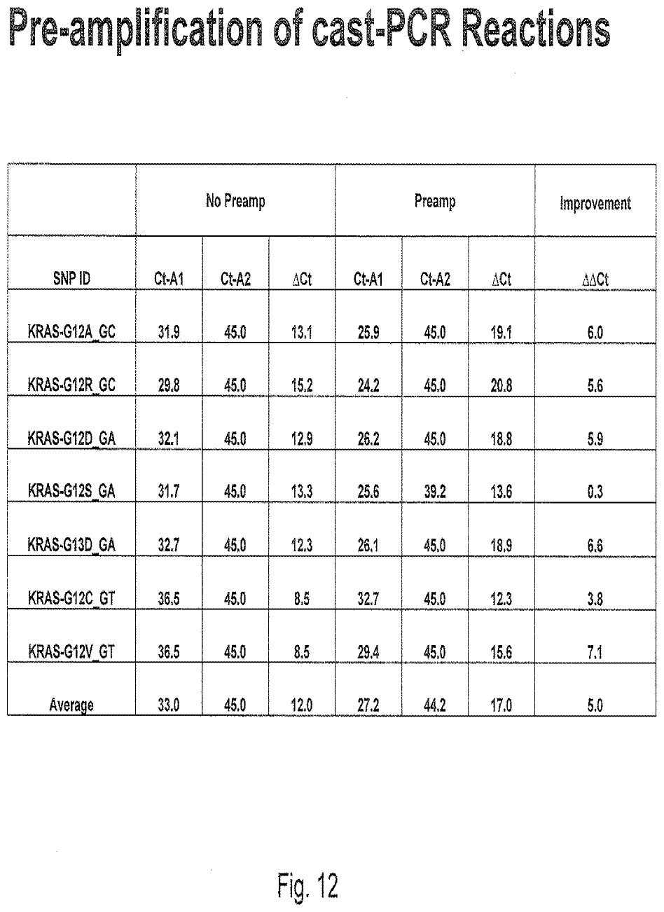

As shown in FIG. 12, for assays without pre-amplification the average ΔCt of the 7 tested KRAS mutations was 12.0, whereas for assays using pre-amplification the average ΔCt was 17.0. Thus, the ΔΔCt between the two gave about a 5 fold improvement for the pre-amplified reactions over those without pre-amplification.

In an ideal situation (where PCR efficiency=100%), the copy number of the target gene increases about 1000 fold in 10 cycles. Under these conditions, if the starting copy number in a pre-amplification reaction is 0.1 ng/μL (or approximately 33 copies/μL), then after 10 cycles the copy number increases to approximately 33,000 copies/μL. In the example above, the copy number of a 20 fold diluted pre-amplification product is estimated to be approximately 1,650 copies/μL Therefore, after adding 1 μL of 1,650 copies/μL diluted pre-amplification product into a cast-PCR reaction (final volume of 10 μL), the concentration of the cast-PCR products is approximately 165 copies/μL Based on this estimation, 10 μL of pre-amplification products from 1 ng/0_, genomic DNA can be diluted by as much as 200 fold, and still provide up to 2000 μL of nucleic acid template for use in subsequent cast-PCR reactions.

### Example 12

### Effect of Tailed ASP on Cast-PCR Specificity

Assays were performed using the general cast-PCR schema and reaction conditions indicated above (see section II of Examples), using 1 million copies of plasmid DNA containing various SNP target sequences (see Table 10). Assay primers and probes were designed according to the sequences shown in FIG. 19A-D. For each SNP analyzed, the blocker probes, locus-specific probes and locus-specific primers were the same and only the allele-specific primers varied (e.g., tailed or non-tailed).

TABLE 10
Plasmid SNP Sequences (SEQ ID NOS 43-78,
respectively, in order of appearance)
(target alleles are indicated in brackets).

| SNP ID | Sequence |
|---|---|
| BRAF-1799TA_TP | ATATTTCTTCATGAAGACCTCACAGTAAAAA |
| | TAGGTGATTTTGGTCTAGCTACAG[T/A]GA |
| | AATCTCGATGGAGTGGGTCCCATCAGTTTGA |
| | ACAGTTGTCTGGATCCATTTTG |
| CTNNB1-121AG_TP | GTATCCACATCCTCTTCCTCAGGATTGCCTT |
| | TACCACTCAGAGAAGGAGCTGTGG[A/G]AG |
| | TGGCACCAGAATGGATTNCAGAGTNCAGGTA |
| | AGACTGTTGCTGCCAGTGACTA |
| CTNNB1-134CT_TP | CAGGACTTGGGAGGTATCCACATCCTCTTCC |
| | TCAGGATTGCCTTTACCACTCAGA[C/T]AA |
| | GGAGCTGTGGTAGTGGCACCAGAATGGATTN |
| | CAGAGTNCAGGTAAGACTGTTG |
| EGFR-2369CT_TP | CCCACGTGTGCCGCCTGCTGGGCATCTGCCT |
| | CACCTCCACCGTGCAGCTCATCA[C/T]GCA |
| | GCTCATGCCCTTCGGCTGCCTCCTGGACTAT |
| | GTCCGGGAACACAAAGACAATA |
| EGFR-2573TG_TP | TTACTTTGCCTCCTTCTGCATGGTATTCTTT |
| | CTCTTCCGCACCCAGCAGTTTGGCC[T/G]G |
| | CCCAAAATCTGTGATCTTGACATGCTGCGGT |
| | GTTTTCACCAGTACGTTCCTGG |
| KRAS-176CG_TP | AGGAAGCAAGTAGTAATTGATGGAGAAACCT |
| | GTCTCTTGGATATTCTCGACACAG[C/G]AG |
| | GTCANGAGGAGTACAGTGCAATGAGGGACCA |
| | GTACATGAGGACTGGGGAGGGC |
| KRAS-183AC_TP | AAGTAGTAATTGATGGAGAAACCTGTCTCTT |
| | GGATATTCTCGACACAGCAGGTCA[A/C]GA |
| | GGAGTACAGTGCAATGAGGGACCAGTACATG |
| | AGGACTGGGGAGGGCTTTCTTT |
| KRAS-34GA_TP | CCACAAAATGATTCTGAATTAGCTGTATCGT |
| | CAAGGCACTCTTGCCTACGCCAC[G/A]AGC |
| | TCCAACTACCACAAGTTTATATTCAGTCATT |
| | TTCAGCAGGCCTTATAATAAAA |
| KRAS-35GA_TP | TCCACAAAATGATTCTGAATTAGCTGTATCG |
| | TCAAGGCACTCTTGCCTACGCCA[G/A]CAG |
| | CTCCAACTACCACAAGTTTATATTCAGTCAT |
| | TTTCAGCAGGCCTTATAATAAA |
| KRAS-3GA_TP | ATTATAAGGCCTGCTGAAAATGACTGAATAT |
| | AAACTTGTGGTAGTTGGAGCTGGTG[G/A]C |
| | GTAGGCAAGAGTGCCTTGACGATACAGCTAA |
| | TTCAGAATCATTTTGTGGACGA |
| NRAS-181CA_TP | AACAAGTGGTTATAGATGGTGAAACCTGTTT |
| | GTTGGACATACTGGATACAGCTGGA[C/A]A |
| | AGAAGAGTACAGTGCCATGAGAGACCAATAC |
| | ATGAGGACAGGCGAAGGCTTCC |
| NRAS-183AT_TP | CAAGTGGTTATAGATGGTGAAACCTGTTTGT |
| | TGGACATACTGGATACAGCTGGACA[A/T]G |
| | AAGAGTACAGTGCCATGAGAGACCAATACAT |
| | GAGGACAGGCGAAGGCTTCCTC |
| BRAF-1799TA_TP | ATATTTCTTCATGAAGACCTCACAGTAAAAA |
| | TAGGTGATTTTGGTCTAGCTACAG[T/A]GA |
| | AATCTCGATGGAGTGGGTCCCATCAGTTTGA |
| | ACAGTTGTCTGGATCCATTTTG |
| CTNNB1-121AG_TP | GTATCCACATCCTCTTCCTCAGGATTGCCTT |
| | TACCACTCAGAGAAGGAGCTGTGG[A/G]AG |
| | TGGCACCAGAATGGATTNCAGAGTNCAGGTA |
| | AGACTGTTGCTGCCAGTGACTA |
| CTNNB1-134CT_TP | CAGGACTTGGGAGGTATCCACATCCTCTTCC |
| | TCAGGATTGCCTTTACCACTCAGA[C/T]AA |
| | GGAGCTGTGGTAGTGGCACCAGAATGGATTN |
| | CAGAGTNCAGGTAAGACTGTTG |
| EGFR-2369CT_TP | CCCACGTGTGCCGCCTGCTGGGCATCTGCCT |
| | CACCTCCACCGTGCAGCTCATCA[C/T]GCA |
| | GCTCATGCCCTTCGGCTGCCTCCTGGACTAT |
| | GTCCGGGAACACAAAGACAATA |
| EGFR-2573TG_TP | TTACTTTGCCTCCTTCTGCATGGTATTCTTT |
| | CTCTTCCGCACCCAGCAGTTTGGCC[T/G]G |
| | CCCAAAATCTGTGATCTTGACATGCTGCGGT |
| | GTTTTCACCAGTACGTTCCTGG |
| KRAS-176CG_TP | AGGAAGCAAGTAGTAATTGATGGAGAAACCT |
| | GTCTCTTGGATATTCTCGACACAG[C/G]AG |
| | GTCANGAGGAGTACAGTGCAATGAGGGACCA |
| | GTACATGAGGACTGGGGAGGGC |
| KRAS-183AC_TP | AAGTAGTAATTGATGGAGAAACCTGTCTCTT |
| | GGATATTCTCGACACAGCAGGTCA[C/G]GA |
| | GGAGTACAGTGCAATGAGGGACCAGTACATG |
| | AGGACTGGGGAGGGCTTTCTTT |
| KRAS-34GA_TP | CCACAAAATGATTCTGAATTAGCTGTATCGT |
| | CAAGGCACTCTTGCCTACGCCAC[G/A]AGC |
| | TCCAACTACCACAAGTTTATATTCAGTCATT |
| | TTCAGCAGGCCTTATAATAAAA |
| KRAS-35GA_TP | TCCACAAAATGATTCTGAATTAGCTGTATCG |
| | TCAAGGCACTCTTGCCTACGCCA[G/A]CAG |
| | CTCCAACTACCACAAGTTTATATTCAGTCAT |
| | TTTCAGCAGGCCTTATAATAAA |
| KRAS-3GA_TP | ATTATAAGGCCTGCTGAAAATGACTGAATAT |
| | AAACTTGTGGTAGTTGGAGCTGGTG[G/A]C |
| | GTAGGCAAGAGTGCCTTGACGATACAGCTAA |
| | TTCAGAATCATTTTGTGGACGA |
| NRAS-181CA_TP | AACAAGTGGTTATAGATGGTGAAACCTGTTT |
| | GTTGGACATACTGGATACAGCTGGA[C/A]A |
| | AGAAGAGTACAGTGCCATGAGAGACCAATAC |
| | ATGAGGACAGGCGAAGGCTTCC |
| NRAS-183AT_TP | CAAGTGGTTATAGATGGTGAAACCTGTTTGT |
| | TGGACATACTGGATACAGCTGGACA[A/T]G |
| | AAGAGTACAGTGCCATGAGAGACCAATACAT |
| | GAGGACAGGCGAAGGCTTCCTC |
| BRAF-1799TA_TP | ATATTTCTTCATGAAGACCTCACAGTAAAAA |
| | TAGGTGATTTTGGTCTAGCTACAG[T/A]GA |
| | AATCTCGATGGAGTGGGTCCCATCAGTTTGA |
| | ACAGTTGTCTGGATCCATTTTG |
| CTNNB1-121AG_TP | GTATCCACATCCTCTTCCTCAGGATTGCCTT |
| | TACCACTCAGAGAAGGAGCTGTGG[A/G]AG |
| | TGGCACCAGAATGGATTNCAGAGTNCAGGTA |
| | AGACTGTTGCTGCCAGTGACTA |
| CTNNB1-134CT_TP | CAGGACTTGGGAGGTATCCACATCCTCTTCC |
| | TCAGGATTGCCTTTACCACTCAGA[C/T]AA |
| | GGAGCTGTGGTAGTGGCACCAGAATGGATTN |
| | CAGAGTNCAGGTAAGACTGTTG |
| EGFR-2369CT_TP | CCCACGTGTGCCGCCTGCTGGGCATCTGCCT |
| | CACCTCCACCGTGCAGCTCATCA[C/T]GCA |
| | GCTCATGCCCTTCGGCTGCCTCCTGGACTAT |
| | GTCCGGGAACACAAAGACAATA |
| EGFR-2573TG_TP | TTACTTTGCCTCCTTCTGCATGGTATTCTTT |
| | CTCTTCCGCACCCAGCAGTTTGGCC[C/T]G |
| | CCCAAAATCTGTGATCTTGACATGCTGCGGT |
| | GTTTTCACCAGTACGTTCCTGG |
| KRAS-176CG_TP | AGGAAGCAAGTAGTAATTGATGGAGAAACCT |
| | GTCTCTTGGATATTCTCGACACAG[T/G]AG |
| | GTCANGAGGAGTACAGTGCAATGAGGGACCA |
| | GTACATGAGGACTGGGGAGGGC |
| KRAS-183AC_TP | AAGTAGTAATTGATGGAGAAACCTGTCTCTT |
| | GGATATTCTCGACACAGCAGGTCA[A/C]GA |
| | GGAGTACAGTGCAATGAGGGACCAGTACATG |
| | AGGACTGGGGAGGGCTTTCTTT |
| KRAS-34GA_TP | CCACAAAATGATTCTGAATTAGCTGTATCGT |
| | CAAGGCACTCTTGCCTACGCCAC[G/A]AGC |
| | TCCAACTACCACAAGTTTATATTCAGTCATT |
| | TTCAGCAGGCCTTATAATAAAA |
| KRAS-35GA_TP | TCCACAAAATGATTCTGAATTAGCTGTATCG |
| | TCAAGGCACTCTTGCCTACGCCA[G/A]CAG |
| | CTCCAACTACCACAAGTTTATATTCAGTCAT |
| | TTTCAGCAGGCCTTATAATAAA |
| KRAS-3GA_TP | ATTATAAGGCCTGCTGAAAATGACTGAATAT |
| | AAACTTGTGGTAGTTGGAGCTGGTG[G/A]C |
| | GTAGGCAAGAGTGCCTTGACGATACAGCTAA |
| | TTCAGAATCATTTTGTGGACGA |
| NRAS-181CA_TP | AACAAGTGGTTATAGATGGTGAAACCTGTTT |
| | GTTGGACATACTGGATACAGCTGGA[C/A]A |
| | AGAAGAGTACAGTGCCATGAGAGACCAATAC |
| | ATGAGGACAGGCGAAGGCTTCC |
| NRAS-183AT_TP | CAAGTGGTTATAGATGGTGAAACCTGTTTGT |
| | TGGACATACTGGATACAGCTGGACA[A/T]G |
| | AAGAGTACAGTGCCATGAGAGACCAATACAT |
| | GAGGACAGGCGAAGGCTTCCTC |

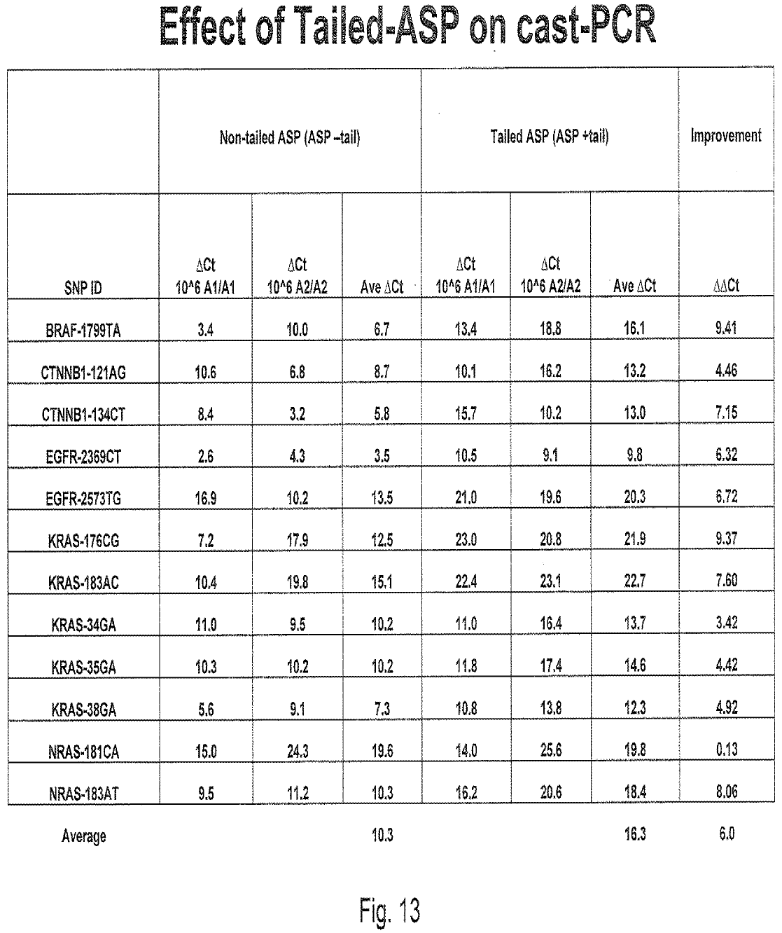

The results of cast-PCR using non-tailed ASP and cast-PCR using tailed-ASP are summarized in FIG. 13. In the cast-PCR reactions having no tailed primers, the average ΔCt of the 12 tested mutations was 10.3, whereas in the cast-PCR reactions with tailed primers the average ΔCt of 12 tested assays is 16.3. Thus, the average ΔΔCt between cast-PCR comprising tailed ASP versus cast-PCR comprising non-tailed ASP was about 6.0, which is about a 64 fold improvement in specificity for reactions comprising ASP+tail primers.

### Example 13

### Comparison of Cast-PCR and ASB-PCR

Allelic discrimination for assays using cast-PCR methods was compared to assays using other Allele-Specific PCR with a Blocking reagent (ASB-PCR) methods (see, e.g., Morlan et al., 2009).

cast-PCR assays were performed using the general schema and reaction conditions indicated above, using 1 million copies of plasmid DNA containing various SNP target sequences (see Table 10). Assays were performed using the general experimental design and reaction conditions indicated above (see section II of Examples). Assay primers and probes were designed according to the sequences shown in FIG. 19B-D.

ASB-PCR assays were performed using 1 million copies of plasmid DNA containing various SNP target sequences (see Table 10), 900 nM non-tailed allele-specific primers (Tm 58˜62° C.), 3600 nM allele-specific phosphate blocker (Tm 58˜62° C.), 200 nM locus-specific TaqMan probe (Tm 70˜74° C.), and 900 nM locus-specific primers (Tm 60˜63° C.).

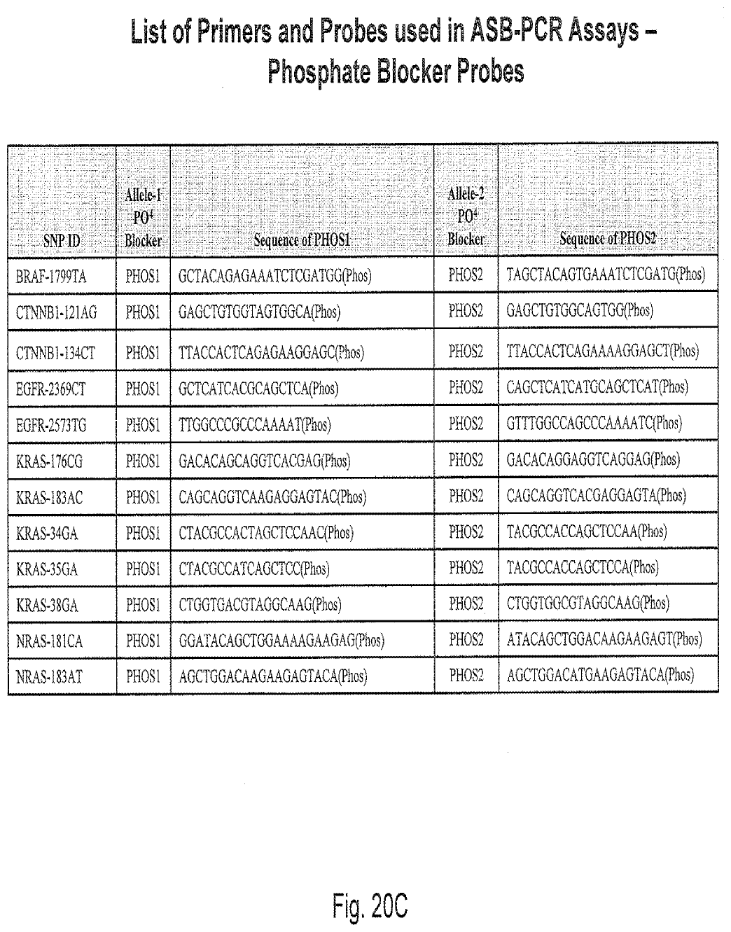

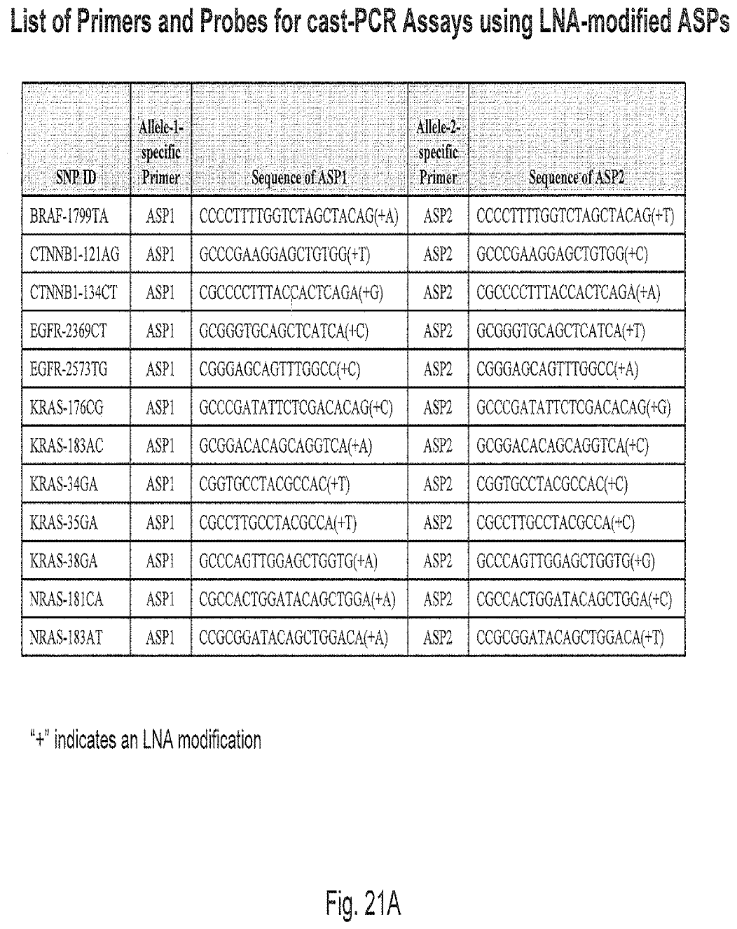

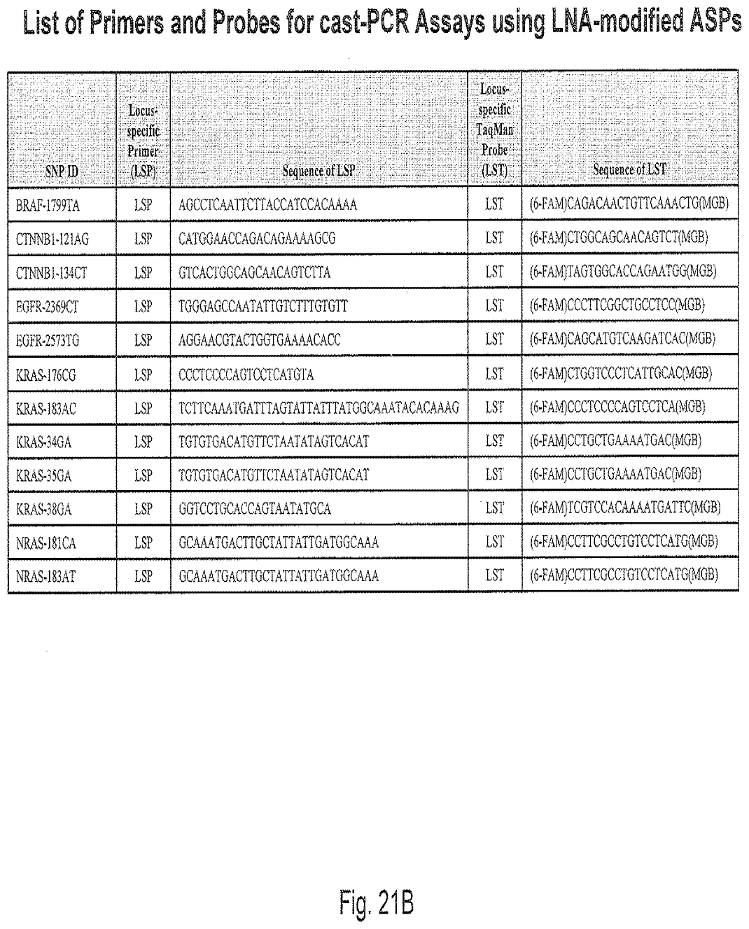

ASB-PCR assay primers and probes were designed according to the sequences shown in FIG. 20A-C. The ASB-PCR reactions were incubated in a 384-well plate at 95° C. for 10 minutes, followed by 50 cycles of at 92° C. for 20 seconds, 60° C. for 45 seconds. All reactions were run in 4 replications in an ABI PRISM 7900HT® Sequence Detection System, according to the manufacturer's instructions.

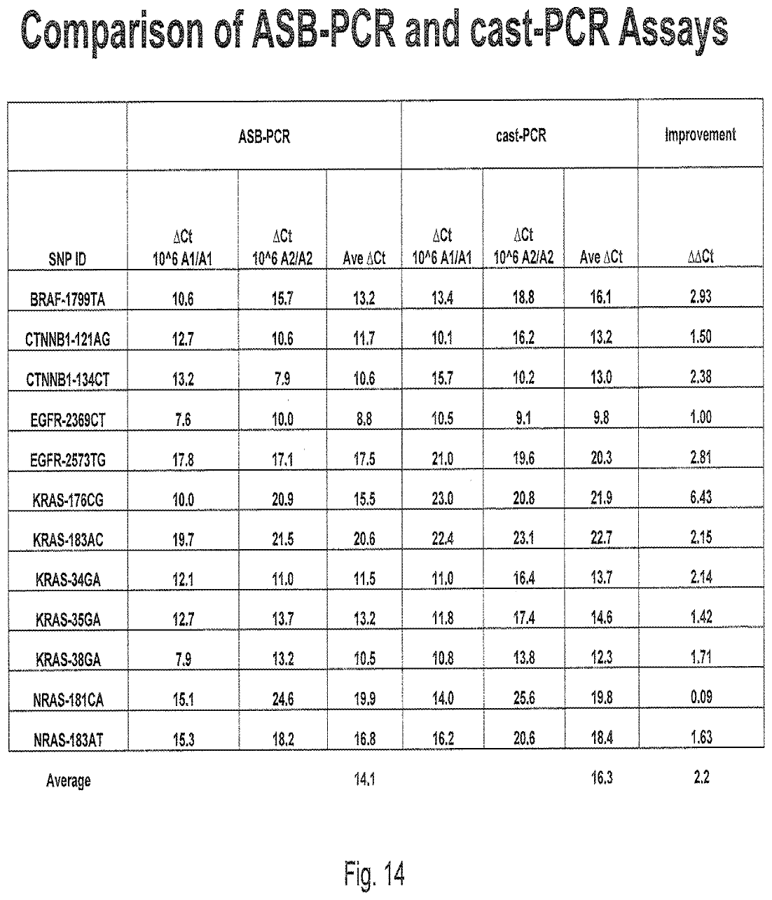

The results for this example are summarized in FIG. 14. In the ASB-PCR assays, the average ΔCt of 12 different mutations was 14.1. In cast-PCR assays, the average ΔCt of the same 12 mutations was 16.3. The ΔΔCt between ASB-PCR and cast-PCR was 2.2, which indicates that the specificity of cast-PCR was approximately 4.6 fold higher than that of the ASB-PCR assay.

### Example 14

### Comparison of MGB and Phosphate Blocker Probes in Cast-PCR

The use of MGB blocker probes was compared to the use of other types of blocker probes, such as PO₄ blocker probes (e.g., Morlan et al., 2009), in cast-PCR assays.

All assays were performed using the general cast-PCR schema and reaction conditions indicated above (see Section II in Examples), using 1 million copies of plasmid DNA containing various SNP target sequences (see Table 10), except that reactions contained either 150 nm allele-specific MGB blocker probes or 150 nm allele-specific 3′-phosphate blocker probes. Assay primers and probes were designed according to the sequences shown in FIG. 19B-D (for cast-PCR using MGB blocker probes) or FIGS. 19B-C and FIG. 20C (for cast-PCR using phosphate blocker probes; “PHOS1” to block allele-1 and “PHOS2” to block allele-2).

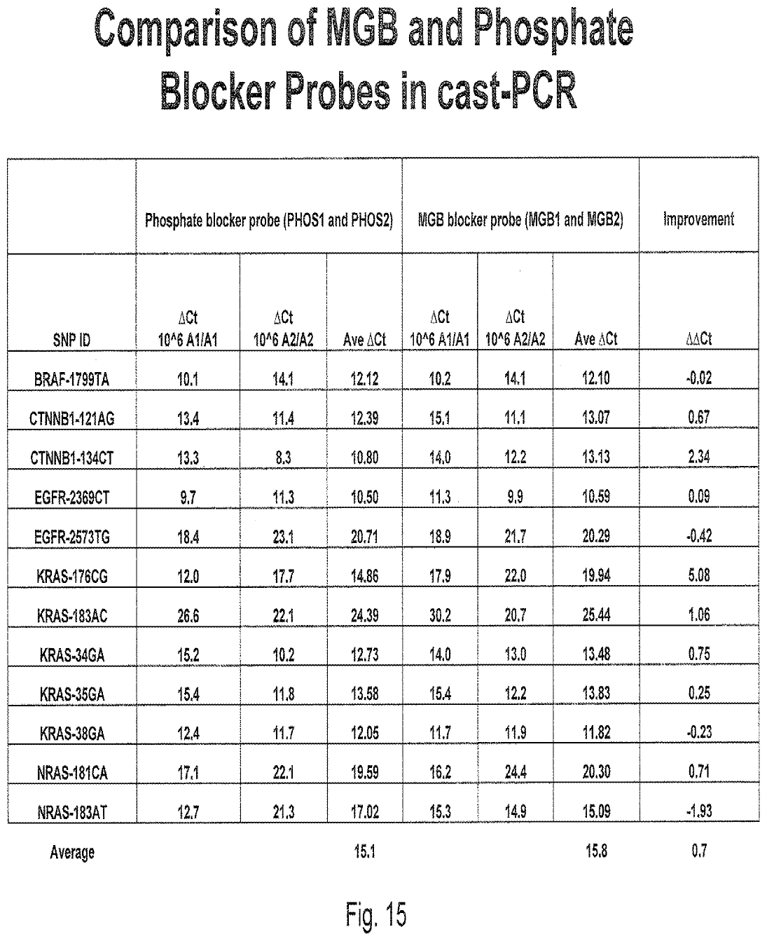

The results of assays with phosphate blocker probes or with MGB blocker probes are summarized in FIG. 15. In cast-PCR assays performed using phosphate blocker probes the average ΔCt of 12 different mutations was 15.1. In comparison, the average ΔCt for the same 12 mutations using cast-PCR assays performed with MGB blocker probes was slightly higher and gave a ΔCt of 15.8.

### Example 15

### Improving the Specificity of Cast-PCR Using LNA Modified ASP

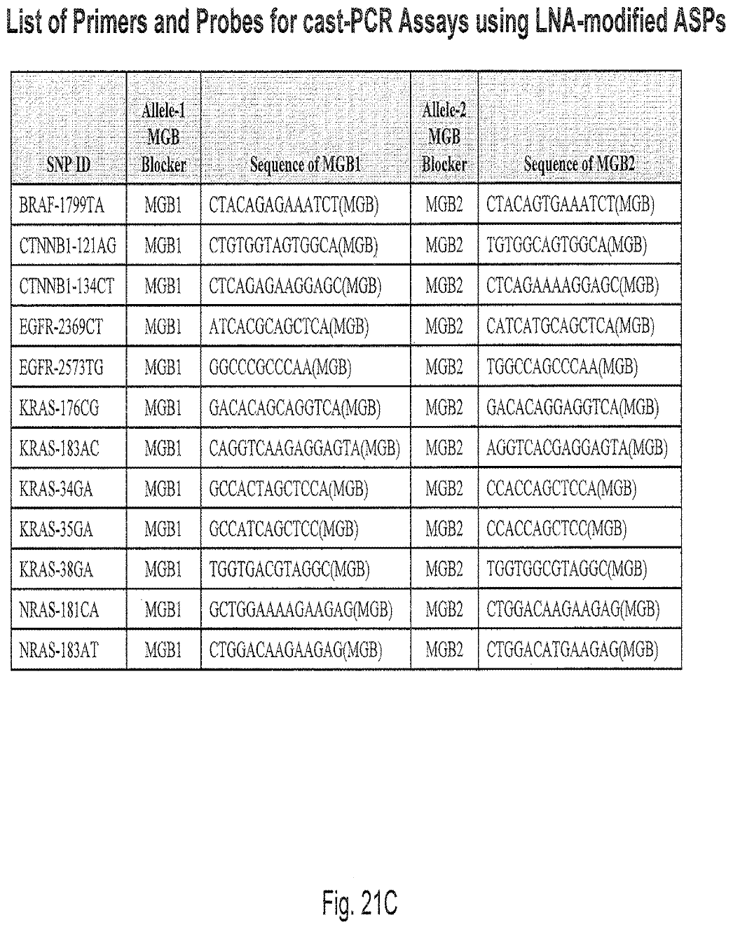

LNA-modified cast-PCR assays were performed using the general experimental design and reaction conditions indicated above (see Section II in Examples), using 0.5 ng/μL of genomic DNA. Assay primers and probes were designed according to the sequences shown in FIGS. 21A-C For each SNP analyzed, the blocker probes, locus-specific probes and locus-specific primers were the same and only the allele-specific primers varied (i.e., with or without an LNA-modification at the 3′end).

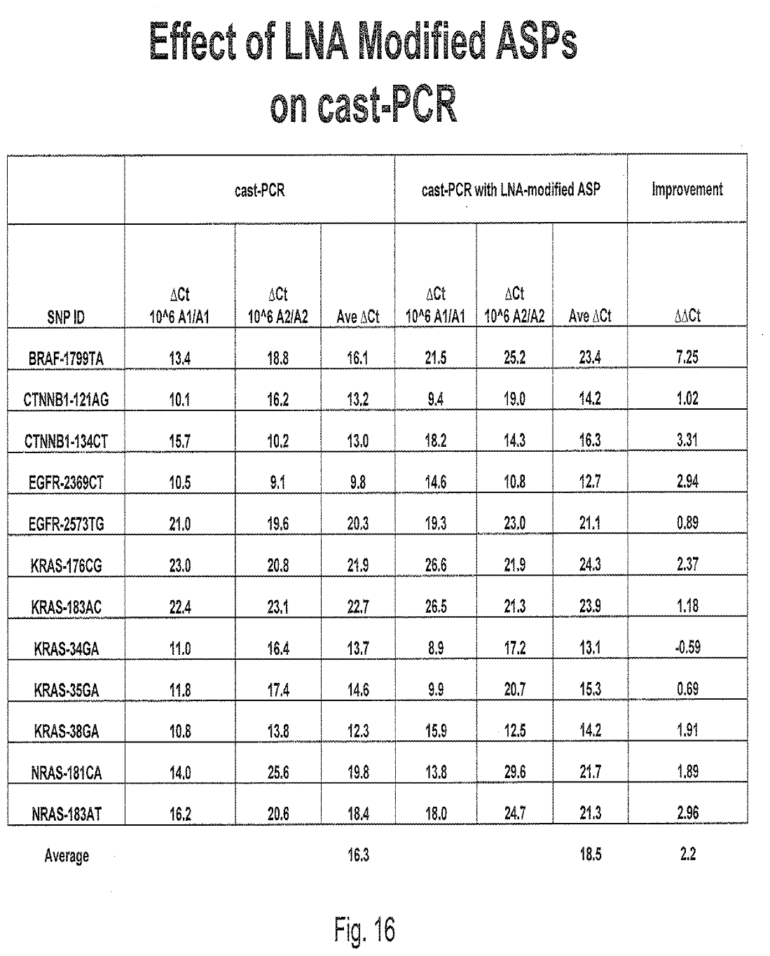

The effect of LNA modification of the ASP on the specificity of cast-PCR is summarized in FIG. 16. For the 12 cast-PCR assays performed using LNA-modified allele-specific primers, the average ΔCt was 16.3. In comparison, the average ΔCt for the same 12 mutations using cast-PCR assays performed allele-specific primers having no modifications the ΔCt was noticeably higher at 18.5. Based on the ΔΔCt the assay specificity increased by approximately 4 fold for those assays that used LNA-modified allele-specific primers.

### Example 16

### Improving the Specificity of Cast-PCR Using Other Modified ASP

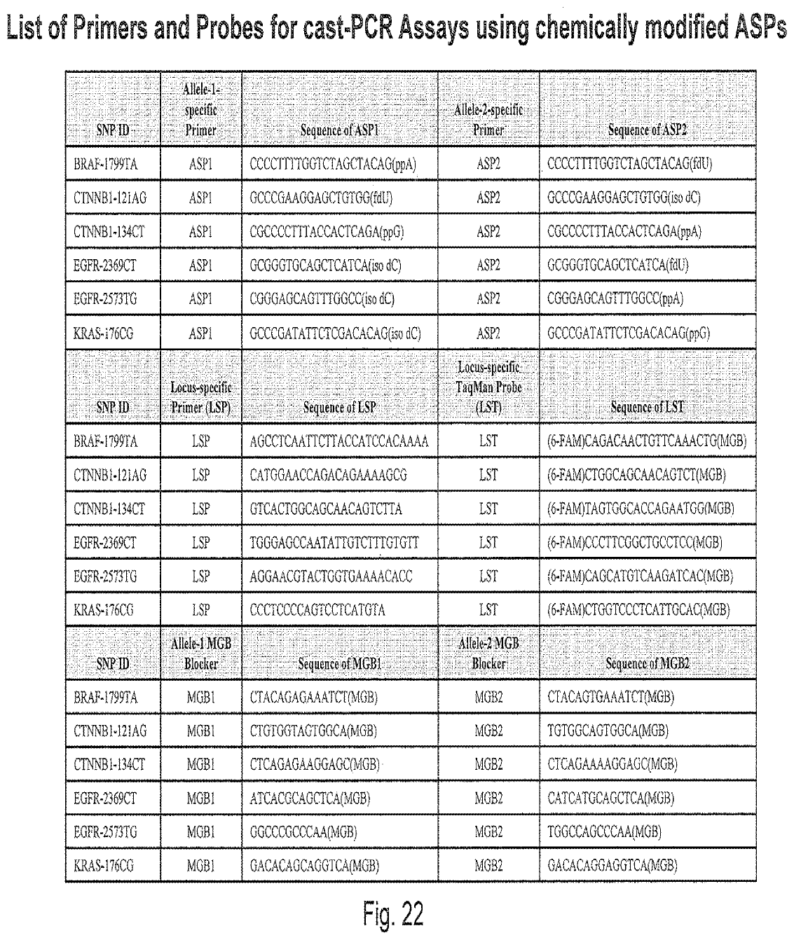

cast-PCR assays using other chemically-modified ASPs were performed using the general experimental design and reaction conditions indicated above conditions indicated above (see Section II in Examples), performed in the presence of 1 million copies of plasmid DNA containing various SNP target sequences (see Table 10). Assay primers and probes were designed according to the sequences shown in FIG. 22. For each SNP analyzed, the blocker probes, locus-specific probes and locus-specific primers were the same and only the allele-specific primers varied (i.e., with or without chemical modifications, i.e., ppA, ppG, iso dC or fdU, at the 3′ end).

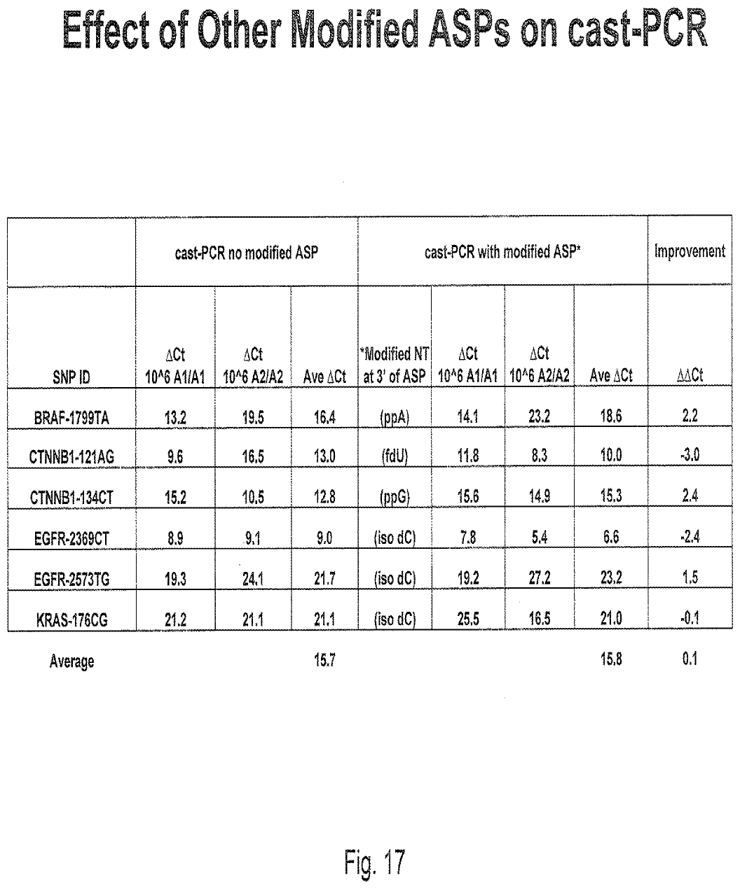

The results of cast-PCR assays using unmodified ASP and cast-PCR assays with modified ASP are summarized in FIG. 17. As shown, allele-specific primers having pyrophosphate modifications (ppA or ppG) at their 3′-ends increased ΔCt by 2-3, which is approximately a 4-6 fold increase in assay specificity.

It is noted that, as used in this specification and the appended claims, the singular forms “a,” “an,” and “the,” include plural referents unless expressly and unequivocally limited to one referent. The use of “or” means “and/or” unless stated otherwise. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

The section headings used herein are for organizational purposes only and are not to be construed as limiting the subject matter described. All documents, or portions of documents, cited in this application, including but not limited to patents, patent applications, articles, books, and treatises are hereby expressly incorporated by reference in their entirety for any purpose. In the event that one or more of the incorporated documents defines a term that contradicts that term's definition in this application, this application controls.

All references cited herein, including patents, patent applications, papers, text books, and the like, and the references cited therein, to the extent that they are not already, are hereby incorporated by reference in their entirety. In the event that one or more of the incorporated literature and similar materials differs.

1. A method for detecting a first allelic variant and a second allelic variant of a target sequence in a nucleic acid sample suspected of comprising at least two allelic variants of the target sequence, the method comprising:
a) performing a multiplex pre-amplification reaction on the nucleic acid sample, the multiplex pre-amplification reaction including a first allele-specific primer, a second allele-specific primer and at least one reverse locus-specific primer,
wherein the first allele-specific primer includes a first allele-specific nucleotide portion that is complementary to the first allelic variant of the target sequence and the second allele-specific primer includes a second allele-specific nucleotide portion that is complementary to the second allelic variant of the target sequence;
b) forming a first reaction mixture by combining:
i) the pre-amplified nucleic acid sample;
ii) the first allele-specific primer;
iii) a first allele-specific blocker probe that is complementary to a first region of the target sequence comprising the second allelic variant, wherein said first region encompasses a position corresponding to a binding position of the first allele-specific nucleotide portion of the first allele-specific primer, and wherein the first allele-specific blocker probe comprises a first minor groove binder;
iv) a first locus-specific primer that is complementary to a region of the target sequence that is 3′ from the first allelic variant and on an opposite strand; and
v) a first detector probe;
b) carrying out a first single-plex amplification reaction on the first reaction mixture using the first locus-specific primer and the first allele-specific primer to form a first amplicon;
c) detecting the first amplicon by detecting a change in a detectable property of the first detector probe, thereby detecting the first allelic variant of the target sequence in the nucleic acid sample;
d) forming a second reaction mixture by combining:
i) the pre-amplified nucleic acid sample;
ii) the second allele-specific primer;
iii) a second allele-specific blocker probe that is complementary to a second region of the target sequence comprising the first allelic variant, wherein said second region encompasses a position corresponding to a binding position of the second allele-specific nucleotide portion of the second allele-specific primer, and wherein the second allele-specific blocker probe comprises a second minor groove binder;
iv) a second locus-specific primer that is complementary to a region of the target sequence that is 3′ from the second allelic variant and on an opposite strand; and
v) a second detector probe;
e) carrying out a second single-plex amplification reaction on the second reaction mixture using the second allele-specific primer and the second locus-specific primer, to form a second amplicon; and
f) detecting the second amplicon by detecting a change in a detectable property of the second detector probe, thereby detecting the second allelic variant of the target sequence in the nucleic acid sample.

2. The method of claim 1, further comprising using the change in the detectable property of the first detector probe to quantitate the first allelic variant.

3. (canceled)

4. The method of claim 1, further comprising comparing the change in the detectable property of the first detector probe in the first reaction mixture to the change in the detectable property of the second detector probe in the second reaction mixture.

5. The method of claim 1, wherein at least one of the first allele-specific primer, the second allele-specific primer, the first allele-specific blocker probe or the second allele-specific blocker probe comprises at least one modified base.

6. The method of claim 5, wherein said modified base is an 8-aza-7-deaza-dN (ppN) base analog, where N is adenine (A), cytosine (C), guanine (G), or thymine (T).

7. The method of claim 5, wherein said modified base is a locked nucleic acid (LNA) base.

8. The method of claim 5, wherein said modified base is a fdU or iso dC base.

9. The method of claim 5, wherein said modified base is any modified base that increases the Tm between matched and mismatched target sequences or nucleotides.

10. The method of claim 5, wherein said modified base is located at (a) the 3′-end, (b) the 5′-end, (c) at an internal position or at any combination of (a), (b) or (c) within at least one of the first allele-specific primer, the second allele-specific primer, the first allele-specific blocker probe or the second allele-specific blocker probe.

11. The method of claim 5, wherein the specificity of said detecting is improved by at least 2 fold when said modified base is included in at least one of the first allele-specific primer, the second allele-specific primer, the first allele-specific blocker probe or the second allele-specific blocker probe as compared to when it is not.

12. (canceled)

13. The method of claim 1, wherein the specificity of detecting the first amplicon and/or the second amplicon is improved by at least 2 fold as compared to the specificity of detecting an allelic variant in a nucleic acid sample using ASB-PCR methods.

14. The method of claim 1, wherein the first single-plex and/or second single-plex amplification reaction comprises a 2-stage cycling protocol.

15. The method of claim 14, wherein the number of cycles in the first stage of said 2-stage cycling protocol comprises fewer cycles than the number of cycles used in the second stage.

16. The method of claim 14, wherein said number of cycles in the first stage is about 90% fewer cycles than said number of cycles in the second stage.

17. The method of claim 14, wherein said number of cycles in the first stage is between 3-7 cycles and said number of cycles in the second stage is between 42-48 cycles.

18. The method of claim 14, wherein a first annealing/extension temperature used during the first cycling stage of said 2-stage cycling protocol is between 1-3 2 C lower than a second annealing/extension temperature used during the second stage.

19. The method of claim 14, wherein a first annealing/extension temperature used during the first cycling stage of said 2-stage cycling protocol is between 56-59° C. and a second annealing/extension temperature used during said second stage is between 60-62° C.

20. (canceled)

21. (canceled)

22. (canceled)

23. The method of claim 1, wherein said multiplex amplification reaction further comprises a plurality of allele-specific blocker probes.

24. The method of claim 1, wherein said multiplex amplification reaction is carried out for a number of cycles suitable to keep the reaction within the linear phase of amplification.

25-46. (canceled)

47. The method of claim 1, further comprising using the change in the detectable property of the second detector probe to quantitate the second allelic variant.
